(19) United States
(12) Patent Application Publication
(10) Pub. No.: US 2012/0135033 A1
(43) Pub. Date: May 31, 2012

(54) MULTIPLE DELIVERY SYSTEM FOR HETEROLOGOUS ANTIGENS

(76) Inventor: ANU WALLECHA, YARDLEY, PA (US)

(21) Appl. No.: 13/290,783

(22) Filed: Nov. 7, 2011

Related U.S. Application Data
(63) Continuation-in-part of application No. 12/993,380, filed on Feb. 7, 2011.
(60) Provisional application No. 61/071,792, filed on May 19, 2008.

(51) Int. Cl. A61K 39/00; C12N 15/74; C12N 1/21; C40B 40/06; C12N 15/12; A61P 35/00

(52) U.S. Cl. 424/200.1; 536/23.5; 435/252.3; 506/16; 435/476

(57) ABSTRACT

The invention is directed to an episomal recombinant nucleic acid encoding at least two heterologous antigens each fused to a PEST-endogenous polypeptide, vaccines comprising the same, methods of preparing same, and methods of inducing an immune response, and treating, inhibiting, or suppressing cancer or tumors comprising administering the same.

### CROSS-REFERENCE

This application is a Continuation-In-Part of U.S. application Ser. No. 12/993,380, filed Feb. 7, 2011, which is a national phase of PCT/US09/44538, International Filing Date May 19, 2009, which claims priority to U.S. Ser. No. 61/071,792, filed May 19, 2008, each of which is hereby incorporated by reference in its entirety.

### FIELD OF INVENTION

The invention is directed to an episomal recombinant nucleic acid encoding at least two heterologous antigens each fused to a PEST-endogenous polypeptide, vaccines comprising the same, methods of preparing same, and methods of inducing an immune response, and treating, inhibiting, or suppressing cancer or tumors comprising administering the same.

### BACKGROUND OF THE INVENTION

A great deal of pre-clinical evidence and early clinical trial data suggests that the anti-tumor capabilities of the immune system can be harnessed to treat patients with established cancers. The vaccine strategy takes advantage of tumor antigens associated with various types of cancers. Immunizing with live vaccines such as viral or bacterial vectors expressing a tumor-associated antigen is one strategy for eliciting strong CTL responses against tumors.

Listeria monocytogenes (Lm) is a gram positive, facultative intracellular bacterium that has direct access to the cytoplasm of antigen presenting cells, such as macrophages and dendritic cells, largely due to the pore-forming activity of listeriolysin-O (LLO). LLO is secreted by Lm following engulfment by the cells and perforates the phagolysosomal membrane, allowing the bacterium to escape the vacuole and enter the cytoplasm. LLO is very efficiently presented to the immune system via MHC class I molecules. Furthermore, Lm-derived peptides also have access to MHC class II presentation via the phagolysosome.

Cancer is a complex disease and combined therapeutic approaches are more likely to succeed. Not only tumor cells, but also the microenvironment that supports tumor growth, must be targeted to maximize the therapeutic efficacy. Most immunotherapies focus on single antigens to target tumor cells and therefore they have shown limited success against human cancers. A single therapeutic agent capable of targeting tumor cells and tumor microenvironment simultaneously would have an advantage over other immunotherapeutic approaches, especially if it results in a synergistic anti-tumor effect.

### SUMMARY OF THE INVENTION

In one embodiment, the present invention relates to a recombinant nucleic acid sequence comprising a first and at least a second open reading frame each encoding a first and at least a second polypeptide, wherein the first and the second polypeptide each comprise a heterologous antigen or a functional fragment thereof fused to an endogenous PEST-containing polypeptide.

In one embodiment, the present invention relates to a recombinant Listeria strain comprising an episomal recombinant nucleic acid molecule, the nucleic acid molecule comprising a first and at least a second open reading frame each encoding a first and at least a second polypeptide, wherein the first and the at least second polypeptide each comprise a heterologous antigen or a functional fragment thereof fused to an endogenous PEST-containing polypeptide.

In another embodiment, the present invention relates to a recombinant Listeria strain comprising a first integrated recombinant nucleic acid molecule comprising a first open reading frame encoding a polypeptide wherein the polypeptide comprises a heterologous antigenic or a functional fragment thereof, fused to an endogenous PEST-containing polypeptide, wherein the first nucleic acid molecule is integrated into the Listeria genome, and wherein the Listeria strain further comprises an episomal recombinant nucleic acid molecule, the episomal nucleic acid molecule comprising a first and at least a second open reading frame each encoding a first and at least a second polypeptide, wherein the first and the at least second polypeptide each comprise a heterologous antigen or a functional fragment thereof fused to an endogenous PEST-containing polypeptide.

In another embodiment, the present invention relates to a recombinant Listeria strain comprising at least one episomal recombinant nucleic acid molecule, the nucleic acid molecules comprising a first and at least a second open reading frame each encoding a first and at least a second polypeptide, wherein the first and the at least second polypeptide each comprise a heterologous antigen or a functional fragment thereof fused to an endogenous PEST-containing polypeptide, wherein the nucleic acids further comprise an open reading frame encoding a plasmid replication control region.

In another embodiment, the present invention relates to a method of inducing an immune response to an antigen in a subject comprising administering to the subject a composition comprising a recombinant Listeria strain comprising an episomal recombinant nucleic acid molecule, the nucleic acid molecule comprising a first and at least a second open reading frame each encoding a first and at least a second polypeptide, wherein the first and the at least second polypeptide each comprise a heterologous antigen or a functional fragment thereof fused to an endogenous PEST-containing polypeptide.

In another embodiment, the present invention relates to a method of producing a recombinant Listeria strain comprising an episomal expression plasmid comprising a first and at least a second nucleic acid encoding a first and at least a second polypeptide, wherein the first and the second polypeptide each comprise a heterologous antigen fused to an endogenous PEST-containing polypeptide, the method comprising the steps of a) recombinantly fusing in the plasmid the first and the second nucleic acid encoding the first and the second polypeptide each comprising a first and a second heterologous antigen fused to an endogenous PEST-containing polypeptide; b) transforming the recombinant Listeria with the episomal expression plasmid; and, c) expressing the first, and the at least second antigens under conditions conducive to antigenic expression in the recombinant Listeria strain.

In one embodiment, the present invention relates to a method of producing a recombinant Listeria strain comprising an episomal expression plasmid comprising a first, a second and a third nucleic acid encoding a first a second and a third polypeptide, wherein the first, the second and the third polypeptide each comprise a heterologous antigen fused to an endogenous PEST-containing polypeptide, the method comprising the steps of a) recombinantly fusing in the plasmid the first, the second and the third nucleic acid encoding the first, the second and the third polypeptide each comprising a first, a second and a third heterologous antigen fused to an endogenous PEST-containing polypeptide; b) transforming the recombinant Listeria with the episomal expression plasmid; and, c) expressing the first, the second and the third antigens under conditions conducive to antigenic expression in the recombinant Listeria strain.

In one embodiment, the present invention relates to a method of producing a recombinant Listeria strain comprising an integrated first nucleic acid, and an episomal expression plasmid comprising at least a second nucleic acid each encoding a first, and at least a second, a wherein the first, and at least the second polypeptides each comprise a heterologous antigen fused to an endogenous PEST-containing polypeptide, the method comprising the steps of a) integrating the first nucleic acid encoding the first polypeptide comprising a first heterologous antigen fused to an endogenous PEST-containing polypeptide into the recombinant Listeria's genome; b) recombinantly fusing in the plasmid the at least second encoding the second comprising a heterologous antigen fused to an endogenous PEST-containing polypeptide; c) transforming the recombinant Listeria with the episomal expression plasmid; and, d) expressing the first, and the at least second antigens under conditions conducive to antigenic expression in the recombinant Listeria strain.

In one embodiment, the present invention relates a method of producing a recombinant Listeria strain comprising an integrated first nucleic acid, and an episomal expression plasmid comprising a second, and a third nucleic acid each encoding a first, a second, and a third polypeptide, wherein the first, second and third polypeptides each comprise a heterologous antigen fused to an endogenous PEST-containing polypeptide, the method comprising the steps of a) integrating the first nucleic acid encoding the first polypeptide comprising a first heterologous antigen fused to an endogenous PEST-containing polypeptide into the recombinant Listeria's genome; b) recombinantly fusing in the plasmid the second and the third nucleic acid encoding the second and the third polypeptide each comprising a second and a third heterologous antigen fused to an endogenous PEST-containing polypeptide; c) transforming the recombinant Listeria with the episomal expression plasmid; and, d) expressing the first, second, and third antigens under conditions conducive to antigenic expression in the recombinant Listeria strain.

In one embodiment, the present invention relates a method of producing a recombinant Listeria strain comprising at least one episomal expression plasmid comprising a first and at least a second nucleic acid encoding a first and at least a second polypeptide, wherein the first and the at least second polypeptide each comprise a heterologous antigen fused to an endogenous PEST-containing polypeptide, the method comprising the steps of a) recombinantly fusing in each plasmid the first and the at least second nucleic acid encoding the first and the second polypeptide each comprising a first and a second heterologous antigen fused to an endogenous PEST-containing polypeptide; b) transforming the recombinant Listeria with each of the episomal expression plasmid; and, c) expressing the first, and the at least second antigens under conditions conducive to antigenic expression in the recombinant Listeria strain, and wherein if the expression of the first, and the at least second antigens place a metabolic burden on the Listeria, each of the plasmids' replication control region activates and expresses a repressor that represses plasmid replication and represses expression of the first, second, and the third heterologous antigen or fragment thereof from each plasmid represses replication of the plasmid and expression from the first, and the at least second heterologous antigen or fragment thereof.

In one embodiment, the present invention relates to a method of producing at least one recombinant Listeria strain comprising an episomal expression plasmid comprising a first, second, and third nucleic acid encoding a first, second and third polypeptide, wherein the first, second and third polypeptide comprise a heterologous antigen fused to an endogenous PEST-containing polypeptide, the method comprising the steps of a) recombinantly fusing in each of the plasmids the first, second and third nucleic acid encoding the first, second and third polypeptide comprising a first, second and third heterologous antigen fused to an endogenous PEST-containing; b) transforming the recombinant Listeria with each of the episomal expression plasmids; and, c) expressing the first, the second, and the third antigens under conditions conducive to antigenic expression in the recombinant Listeria strain, and wherein if the expression of the first, the second, and the third antigens from each plasmid place a metabolic burden on the Listeria, each of the plasmids' replication control region activates and expresses a repressor that represses plasmid replication and represses expression of the first, second, and the third heterologous antigen or fragment thereof from each plasmid.

In one embodiment, the present invention relates to a method of producing a recombinant Listeria strain comprising an integrated first nucleic acid, and at least one episomal expression plasmid comprising a second, and a third nucleic acid each encoding a first, a second, and a third polypeptide, wherein the first, second and third polypeptides each comprise a heterologous antigen fused to an endogenous PEST-containing polypeptide, the method comprising the steps of a) integrating the first nucleic acid encoding the first polypeptide comprising a first heterologous antigen fused to an endogenous PEST-containing polypeptide into the recombinant Listeria's genome; b) recombinantly fusing in each of the plasmids the second and the third nucleic acid encoding the second and the third polypeptide each comprising a second and a third heterologous antigen fused to an endogenous PEST-containing polypeptide; c) transforming the recombinant Listeria with each of the episomal expression plasmids; and, d) expressing the first, the second, and the third antigens under conditions conducive to antigenic expression in the recombinant Listeria strain, and wherein if the expression of the first, the second, and the third antigens from each plasmid place a metabolic burden on the Listeria, each of the plasmids' replication control region activates and expresses a repressor that represses plasmid replication and represses expression of the first, second, and the third heterologous antigen or fragment thereof from each plasmid.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

FIG. 1. (A) Schematic representation of the chromosomal region of the Lmdd-143 and LmddA-143 after klk3 integration and actA deletion; (B) The klk3 gene is integrated into the Lmdd and LmddA chromosome. PCR from chromosomal DNA preparation from each construct using klk3 specific primers amplifies a band of 714 bp corresponding to the klk3 gene, lacking the secretion signal sequence of the wild type protein.

FIG. 2. (A) Map of the pADV134 plasmid. (B) Proteins from LmddA-134 culture supernatant were precipitated, separated in a SDS-PAGE, and the LLO-E7 protein detected by Western-blot using an anti-E7 monoclonal antibody. The antigen expression cassette consists of hly promoter, ORF for truncated LLO and human PSA gene (klk3). (C) Map of the pADV142 plasmid. (D) Western blot showed the expression of LLO-PSA fusion protein using anti-PSA and anti-LLO antibody.

FIG. 3. (A) Plasmid stability in vitro of LmddA-LLO-PSA if cultured with and without selection pressure (D-alanine). Strain and culture conditions are listed first and plates used for CFU determination are listed after. (B) Clearance of LmddA-LLO-PSA in vivo and assessment of potential plasmid loss during this time. Bacteria were injected i.v. and isolated from spleen at the time point indicated. CFUs were determined on BHI and BHI+D-alanine plates.

FIG. 4. (A) In vivo clearance of the strain LmddA-LLO-PSA after administration of 10⁸ CFU in C57BL/6 mice. The number of CFU was determined by plating on BHI/str plates. The limit of detection of this method was 100 CFU. (B) Cell infection assay of J774 cells with 10403S, LmddA-LLO-PSA and XFL7 strains.

FIG. 5. (A) PSA tetramer-specific cells in the splenocytes of naïve and LmddA-LLO-PSA immunized mice on day 6 after the booster dose. (B) Intracellular cytokine staining for IFN-γ in the splenocytes of naïve and LmddA-LLO-PSA immunized mice were stimulated with PSA peptide for 5 h. Specific lysis of EL4 cells pulsed with PSA peptide with in vitro stimulated effector T cells from LmddA-LLO-PSA immunized mice and naïve mice at different effector/target ratio using a caspase based assay (C) and a europium based assay (D). Number of IFNγ spots in naïve and immunized splenocytes obtained after stimulation for 24 h in the presence of PSA peptide or no peptide (E).

FIG. 6. Immunization with LmddA-142 induces regression of Tramp-C1-PSA (TPSA) tumors. Mice were left untreated (n=8) (A) or immunized i.p. with LmddA-142 (1×10⁸ CFU/mouse) (n=8) (B) or Lm-LLO-PSA (n=8) (C) on days 7, 14 and 21. Tumor sizes were measured for each individual tumor and the values expressed as the mean diameter in millimeters. Each line represents an individual mouse.

FIG. 7. (A) Analysis of PSA-tetramer⁺CD8⁺ T cells in the spleens and infiltrating T-PSA-23 tumors of untreated mice and mice immunized with either an Lm control strain or Lm-ddA-LLO-PSA (LmddA-142). (B) Analysis of CD4⁺ regulatory T cells, which were defined as CD25⁺FoxP3⁺, in the spleens and infiltrating T-PSA-23 tumors of untreated mice and mice immunized with either an Lm control strain or Lm-ddA-LLO-PSA.

FIG. 8. (A) Schematic representation of the chromosomal region of the Lmdd-143 and LmddA-143 after klk3 integration and actA deletion; (B) The klk3 gene is integrated into the Lmdd and LmddA chromosome. PCR from chromosomal DNA preparation from each construct using klk3 specific primers amplifies a band of 760 by corresponding to the klk3 gene.

FIG. 9. (A) Lmdd-143 and LmddA-143 secretes the LLO-PSA protein. Proteins from bacterial culture supernatants were precipitated, separated in a SDS-PAGE and LLO and LLO-PSA proteins detected by Western-blot using an anti-LLO and anti-PSA antibodies; (B) LLO produced by Lmdd-143 and LmddA-143 retains hemolytic activity. Sheep red blood cells were incubated with serial dilutions of bacterial culture supernatants and hemolytic activity measured by absorbance at 590 nm; (C) Lmdd-143 and LmddA-143 grow inside the macrophage-like J774 cells. J774 cells were incubated with bacteria for 1 hour followed by gentamicin treatment to kill extracellular bacteria. Intracellular growth was measured by plating serial dilutions of J774 lysates obtained at the indicated time points. Lm 10403S was used as a control in these experiments.

FIG. 10. Immunization of mice with Lmdd-143 and LmddA-143 induces a PSA-specific immune response. C57BL/6 mice were immunized twice at 1-week interval with 1×10⁸ CFU of Lmdd-143, LmddA-143 or LmddA-142 and 7 days later spleens were harvested. Splenocytes were stimulated for 5 hours in the presence of monensin with 1 μM of the PSA₆₅₋₇₄ peptide. Cells were stained for CD8, CD3, CD62L and intracellular IFN-γ and analyzed in a FACS Calibur cytometer.

FIG. 11. Three Lm-based vaccines expressing distinct HMW-MAA fragments based on the position of previously mapped and predicted HLA-A2 epitopes were designed (A). The Lm-tLLO-HMW-MMA₂₁₆₀₋₂₂₅₈ (also referred as Lm-LLO-HMW-MAA-C) strain secretes a ˜62 kDa band corresponding to the tLLO-HMW-MAA₂₁₆₀₋₂₂₅₈ fusion protein (B). C57BL/6 mice (n=15) were inoculated s.c. with B16F10 cells and either immunized i.p. on days 3, 10 and 17 with Lm-tLLO-HMW-MAA₂₁₆₀₋₂₂₅₈ (n=8) or left untreated (n=7). BALB/c mice (n=16) were inoculated s.c. with RENCA cells and immunized i.p. on days 3, 10 and 17 with either Lm-HMW-MAA-C (n=8) or an equivalent dose of a control Lm vaccine. Mice immunized with the Lm-LLO-HMW-MAA-C impeded the growth of established tumors (C). FVB/N mice (n=13) were inoculated s.c. with NT-2 tumor cells and immunized i.p. on days 7, 14 and 21 with either Lm-HMW-MAA-C (n=5) or an equivalent dose of a control Lm vaccine (n=8) Immunization of mice with Lm-LLO-HMW-MAA-C significantly impaired the growth of tumors not engineered to express HMW-MAA, such as B16F10, RENCA and NT-2 (D). Tumor sizes were measured for each individual tumor and the values expressed as the mean diameter in millimeters±SEM. *, P≧0.05, Mann-Whitney test.

FIG. 12 Immunization with Lm-HMW-MAA-C promotes tumor infiltration by CD8⁺ T cells and decreases the number of pericytes in blood vessels. (A) NT-2 tumors were removed and sectioned for immunofluorescence. Staining groups are numbered (1-3) and each stain is indicated on the right. Sequential tissues were either stained with the pan-vessel marker anti-CD31 or the anti-NG2 antibody for the HMW-MAA mouse homolog AN2, in conjunction with anti-CD8α for possible TILs. Group 3 shows isotype controls for the above antibodies and DAPI staining used as a nuclear marker. A total of 5 tumors were analyzed and a single representative image from each group is shown. CD8⁺ cells around blood vessels are indicated by arrows. (B) Sequential sections were stained for pericytes by using the anti-NG2 and anti-alpha-smooth-muscle-cell-actin (α-SMA) antibodies. Double staining/colocalization of these two antibodies (yellow in merge image) are indicative of pericyte staining (top). Pericyte colocalization was quantitated using Image Pro Software and the number of colocalized objects is shown in the graph (bottom). A total of 3 tumors were analyzed and a single representative image from each group is shown. *, P≦0.05, Mann-Whitney test. Graph shows mean±SEM.

FIG. 13. Schematic representation of pAdv134 plasmid and dual plasmid. The restriction sites that will be used for cloning of antigen 1 (Xho I and SpeI) and antigen 2 (XbaI and SacI or BglII) genes are indicated. The black arrow represents the direction of transcription. p15 on and RepR refer to Listeria and E. coli origin of replication. tLLO is truncated Listeriolysin O protein (1-441 aa) and tActA is truncated ActA (1-233 aa) protein. Bacillus-dal gene codes for D-alanine racemase which complements for the synthesis of D-alanine in LmΔdal dat strain.

### DETAILED DESCRIPTION OF THE INVENTION

This invention provides, in one embodiment, a recombinant Listeria strain comprising a bivalent episomal expression vector, the vector comprising a first, and at least a second nucleic acid molecule encoding a heterologous antigenic polypeptide or a functional fragment thereof, wherein the first and the second nucleic acid molecules each encode the heterologous antigenic polypeptide or functional fragment thereof in an open reading frame with an endogenous PEST-containing polypeptide.

In another embodiment, the “functional fragment” is an immunogenic fragment and elicits an immune response when administered to a subject alone or in a vaccine composition provided herein. In another embodiment, a functional fragment has biological activity as will be understood by a skilled artisan and as further provided herein.

In one embodiment, the term “at least second nucleic acid molecule” refers to two or more nucleic acid molecules, alternatively it refers to three, four, five, and so on nucleic acid molecules.

In another embodiment, the recombinant nucleic acid molecule further comprises a third open reading frame encoding a third polypeptide, wherein said third polypeptide comprises a heterologous antigen or a functional fragment thereof fused to an endogenous PEST-containing polypeptide.

In one embodiment, provided herein is a multivalent plasmid that delivers at least two antigens. In another embodiment, the plasmid is a dual plasmid. In another embodiment, provided herein is an episomal recombinant nucleic acid encoding the multivalent plasmid. In another embodiment, the episomal recombinant nucleic acid backbone is encoded by the sequence comprising SEQ ID NO: 30. In another embodiment, the episomal recombinant nucleic acid provided herein is encoded by the sequence consisting of SEQ ID NO: 30. In another embodiment, the episomal recombinant nucleic acid provided herein is encoded by the sequence set forth in SEQ ID NO: 30.

(SEQ ID NO: 30)
ggagtgtatactggcttactatgttggcactgatgagggtgtcagtgaagtgcttcatgtggcaggagaaaaaaggctgcaccggtgcg
tcagcagaatatgtgatacaggatatattccgcttcctcgctcactgactcgctacgctcggtcgttcgactgcggcgagcggaaatggc
ttacgaacggggcggagatttcctggaagatgccaggaagatacttaacagggaagtgagagggccgcggcaaagccgtttttccat
aggctccgcccccctgacaagcatcacgaaatctgacgctcaaatcagtggtggcgaaacccgacaggactataaagataccaggcg
tttccccctggcggctccctcgtgcgctctcctgttcctgcctttcggtttaccggtgtcattccgctgttatggccgcgtttgtctcattccac
gcctgacactcagttccgggtaggcagttcgctccaagctggactgtatgcacgaaccccccgttcagtccgaccgctgcgccttatcc
ggtaactatcgtcttgagtccaacccggaaagacatgcaaaagcaccactggcagcagccactggtaattgatttagaggagttagtctt
gaagtcatgcgccggttaaggctaaactgaaaggacaagttttggtgactgcgctcctccaagccagttacctcggttcaaagagttggt
agctcagagaaccttcgaaaaaccgccctgcaaggcggttttttcgttttcagagcaagagattacgcgcagaccaaaacgatctcaag
aagatcatcttattaatcagataaaatatttctagccctcctttgattagtatattcctatcttaaagttacttttatgtggaggcattaacatttgtt
aatgacgtcaaaaggatagcaagactagaataaagctataaagcaagcatataatattgcgtttcatctttagaagcgaatttcgccaatat
tataattatcaaaagagaggggtggcaaacggtatttggcattattaggttaaaaaatgtagaaggagagtgaaacccatgaaaaaaata
atgctagtttttattacacttatattagttagtctaccaattgcgcaacaaactgaagcaaaggatgcatctgcattcaataaagaaaattcaa
tttcatccatggcaccaccagcatctccgcctgcaagtcctaagacgccaatcgaaaagaaacacgcggatgaaatcgataagtatata
caaggattggattacaataaaaacaatgtattagtataccacggagatgcagtgacaaatgtgccgccaagaaaaggttacaaagatgg
aaatgaatatattgttgtggagaaaaagaagaaatccatcaatcaaaataatgcagacattcaagttgtgaatgcaatttcgagcctaacct
atccaggtgctctcgtaaaagcgaattcggaattagtagaaaatcaaccagatgttctccctgtaaaacgtgattcattaacactcagcatt
gatttgccaggtatgactaatcaagacaataaaatagttgtaaaaaatgccactaaatcaaacgttaacaacgcagtaaatacattagtgg
aaagatggaatgaaaaatatgctcaagcttatccaaatgtaagtgcaaaaattgattatgatgacgaaatggcttacagtgaatcacaatta
attgcgaaatttggtacagcatttaaagctgtaaataatagcttgaatgtaaacttcggcgcaatcagtgaagggaaaatgcaagaagaa
gtcattagttttaaacaaatttactataacgtgaatgttaatgaacctacaagaccttccagatttttcggcaaagctgttactaaagagcagt
tgcaagcgcttggagtgaatgcagaaaatcctcctgcatatatctcaagtgtggcgtatggccgtcaagtttatttgaaattatcaactaatt
cccatagtactaaagtaaaagctgcttttgatgctgccgtaagcggaaaatctgtctcaggtgatgtagaactaacaaatatcatcaaaaat
tcttccttcaaagccgtaatttacggaggttccgcaaaagatgaagttcaaatcatcgacggcaacctcggagacttacgcgatattttga
aaaaaggcgctacttttaatcgagaaacaccaggagttcccattgcttatacaacaaacttcctaaaagacaatgaattagctgttattaaa
aacaactcagaatatattgaaacaacttcaaaagcttatacagatggaaaaattaacatcgatcactctggaggatacgttgctcaattcaa
catttcttgggatgaagtaaattatgatctcgagactagttctagatttatcacgtacccatttccccgcatcttttatttttttaaatactttaggg
aaaaatggtttttgatttgcttttaaaggttgtggtgtagactcgtctgctgactgcatgctagaatctaagtcactttcagaagcatccacaa
ctgactctttcgccacttttctcttatttgcttttgttggtttatctggataagtaaggctttcaagctcactatccgacgacgctatggcttttctt
ctttttttaatttccgctgcgctatccgatgacagacctggatgacgacgctccacttgcagagttggtcggtcgactcctgaagcctcttca
tttatagccacatttcctgtttgctcaccgttgttattattgttattcggacctttctctgcttttgctttcaacattgctattaggtctgctttgttcgt
atttttcactttattcgatttttctagttcctcaatatcacgtgaacttacttcacgtgcagtttcgtatcttggtcccgtatttacctcgcttggctg
ctcttctgttttttcttcttcccattcatctgtgtttagactggaatcttcgctatctgtcgctgcaaatattatgtcggggttaatcgtaatgcagtt
ggcagtaatgaaaactaccatcatcgcacgcataaatctgtttaatcccacttatactccctcctcgtgatacgctaatacaacctttttaga
acaaggaaaattcggccttcattttcactaatttgttccgttaaaaattggattagcagttagttatcttcttaattagctaatataagaaaaaat
attcatgaattattttaagaatatcacttggagaattaatttttctctaacatttgttaatcagttaaccccaactgcttcccaagcttcacccgg
gccactaactcaacgctagtagtggatttaatcccaaatgagccaacagaaccagaaccagaaacagaacaagtaacattggagttag
aaatggaagaagaaaaaagcaatgatttcgtgtgaataatgcacgaaatcattgcttatttttttaaaaagcgatatactagatataacgaa
acaacgaactgaataaagaatacaaaaaaagagccacgaccagttaaagcctgagaaactttaactgcgagccttaattgattaccacc
aatcaattaaagaagtcgagacccaaaatttggtaaagtatttaattactttattaatcagatacttaaatatctgtaaacccattatatcgggtt
tttgaggggatttcaagtctttaagaagataccaggcaatcaattaagaaaaacttagttgattgccttttttgttgtgattcaactttgatcgta
gcttctaactaattaattttcgtaagaaaggagaacagctgaatgaatatcccttttgttgtagaaactgtgcttcatgacggcttgttaaagt
acaaatttaaaaatagtaaaattcgctcaatcactaccaagccaggtaaaagtaaaggggctatttttgcgtatcgctcaaaaaaaagcat
gattggcggacgtggcgttgttctgacttccgaagaagcgattcacgaaaatcaagatacatttacgcattggacaccaaacgtttatcgt
tatggtacgtatgcagacgaaaaccgttcatacactaaaggacattctgaaaacaatttaagacaaatcaataccttctttattgattttgata
ttcacacggaaaaagaaactatttcagcaagcgatattttaacaacagctattgatttaggttttatgcctacgttaattatcaaatctgataaa
ggttatcaagcatattttgttttagaaacgccagtctatgtgacttcaaaatcagaatttaaatctgtcaaagcagccaaaataatctcgcaa
aatatccgagaatattttggaaagtctttgccagttgatctaacgtgcaatcattttgggattgctcgtataccaagaacggacaatgtagaa
ttttttgatcccaattaccgttattctttcaaagaatggcaagattggtctttcaaacaaacagataataagggctttactcgttcaagtctaac
ggttttaagcggtacagaaggcaaaaaacaagtagatgaaccctggtttaatctcttattgcacgaaacgaaattttcaggagaaaaggg
tttagtagggcgcaatagcgttatgtttaccctctctttagcctactttagttcaggctattcaatcgaaacgtgcgaatataatatgtttgagtt
taataatcgattagatcaacccttagaagaaaaagaagtaatcaaaattgttagaagtgcctattcagaaaactatcaaggggctaatagg
gaatacattaccattctttgcaaagcttgggtatcaagtgatttaaccagtaaagatttatttgtccgtcaagggtggtttaaattcaagaaaa
aaagaagcgaacgtcaacgtgttcatttgtcagaatggaaagaagatttaatggcttatattagcgaaaaaagcgatgtatacaagcctta
tttagcgacgaccaaaaaagagattagagaagtgctaggcattcctgaacggacattagataaattgctgaaggtactgaaggcgaatc
aggaaattttctttaagattaaaccaggaagaaatggtggcattcaacttgctagtgttaaatcattgttgctatcgatcattaaattaaaaaa
agaagaacgagaaagctatataaaggcgctgacagcttcgtttaatttagaacgtacatttattcaagaaactctaaacaaattggcagaa
cgccccaaaacggacccacaactcgatttgtttagctacgatacaggctgaaaataaaacccgcactatgccattacatttatatctatgat
acgtgtttgtttttctttgctggctagcttaattgcttatatttacctgcaataaaggatttcttacttccattatactcccattttccaaaaacatac
ggggaacacgggaacttattgtacaggccacctcatagttaatggtttcgagccttcctgcaatctcatccatggaaatatattcatccccc
tgccggcctattaatgtgacttttgtgcccggcggatattcctgatccagctccaccataaattggtccatgcaaattcggccggcaattttc
aggcgttttcccttcacaaggatgtcggtccctttcaattttcggagccagccgtccgcatagcctacaggcaccgtcccgatccatgtgt
ctttttccgctgtgtactcggctccgtagctgacgctctcgccttttctgatcagtttgacatgtgacagtgtcgaatgcagggtaaatgccg
gacgcagctgaaacggtatctcgtccgacatgtcagcagacgggcgaaggccatacatgccgatgccgaatctgactgcattaaaaa
agccttttttcagccggagtccagcggcgctgttcgcgcagtggaccattagattctttaacggcagcggagcaatcagctctttaaagc
gctcaaactgcattaagaaatagcctctttctttttcatccgctgtcgcaaaatgggtaaatacccctttgcactttaaacgagggttgcggt
caagaattgccatcacgttctgaacttcttcctctgtttttacaccaagtctgttcatccccgtatcgaccttcagatgaaaatgaagagaac
cttttttcgtgtggcgggctgcctcctgaagccattcaacagaataacctgttaaggtcacgtcatactcagcagcgattgccacatactcc
gggggaaccgcgccaagcaccaatataggcgccttcaatccctttttgcgcagtgaaatcgcttcatccaaaatggccacggccaagc
atgaagcacctgcgtcaagagcagcctttgctgtttctgcatcaccatgcccgtaggcgtttgctttcacaactgccatcaagtggacatg
ttcaccgatatgttttttcatattgctgacattttcctttatcacggacaagtcaatttccgcccacgtatctctgtaaaaaggttttgtgctcatg
gaaaactcctctcttttttcagaaaatcccagtacgtaattaagtatttgagaattaattttatattgattaatactaagtttacccagttttcacct
aaaaaacaaatgatgagataatagctccaaaggctaaagaggactataccaactatttgttaat.

In one embodiment, the multivalent plasmid backbone comprises at least two nucleic acid sequences encoding at least two antigens. In another embodiment, the recombinant episomal nucleic acid encodes a plasmid backbone sequence and at least two antigens. In another embodiment, the antigens are heterologous antigens to the bacteria host carrying the plasmid. In another embodiment, the antigens are heterologous antigens to the Listeria host carrying the plasmid. In another embodiment, the recombinant episomal nucleic acid sequence encoding the plasmid backbone and at least two heterologous antigens comprises SEQ ID NO: 31. In another embodiment, the recombinant episomal nucleic acid sequence encoding the plasmid backbone and at least two heterologous antigens consists of SEQ ID NO: 31.

(SEQ ID NO: 31)
ggagtgtatactggcttactatgttggcactgatgagggtgtcagtgaagtgcttcatgtggcaggagaaaaaaggctgcaccggtgcg
tcagcagaatatgtgatacaggatatattccgcttcctcgctcactgactcgctacgctcggtcgttcgactgcggcgagcggaaatggc
ttacgaacggggcggagatttcctggaagatgccaggaagatacttaacagggaagtgagagggccgcggcaaagccgtttttccat
aggctccgcccccctgacaagcatcacgaaatctgacgctcaaatcagtggtggcgaaacccgacaggactataaagataccaggcg
tttccccctggcggctccctcgtgcgctctcctgttcctgcctttcggtttaccggtgtcattccgctgttatggccgcgtttgtctcattccac
gcctgacactcagttccgggtaggcagttcgctccaagctggactgtatgcacgaaccccccgttcagtccgaccgctgcgccttatcc
ggtaactatcgtcttgagtccaacccggaaagacatgcaaaagcaccactggcagcagccactggtaattgatttagaggagttagtctt
gaagtcatgcgccggttaaggctaaactgaaaggacaagttttggtgactgcgctcctccaagccagttacctcggttcaaagagttggt
agctcagagaaccttcgaaaaaccgccctgcaaggcggttttttcgttttcagagcaagagattacgcgcagaccaaaacgatctcaag
aagatcatcttattaatcagataaaatatttctagccctcctttgattagtatattcctatcttaaagttacttttatgtggaggcattaacatttgtt
aatgacgtcaaaaggatagcaagactagaataaagctataaagcaagcatataatattgcgtttcatctttagaagcgaatttcgccaatat
tataattatcaaaagagaggggtggcaaacggtatttggcattattaggttaaaaaatgtagaaggagagtgaaacccatgaaaaaaata
atgctagtttttattacacttatattagttagtctaccaattgcgcaacaaactgaagcaaaggatgcatctgcattcaataaagaaaattcaa
tttcatccatggcaccaccagcatctccgcctgcaagtcctaagacgccaatcgaaaagaaacacgcggatgaaatcgataagtatata
caaggattggattacaataaaaacaatgtattagtataccacggagatgcagtgacaaatgtgccgccaagaaaaggttacaaagatgg
aaatgaatatattgttgtggagaaaaagaagaaatccatcaatcaaaataatgcagacattcaagttgtgaatgcaatttcgagcctaacct
atccaggtgctctcgtaaaagcgaattcggaattagtagaaaatcaaccagatgttctccctgtaaaacgtgattcattaacactcagcatt
gatttgccaggtatgactaatcaagacaataaaatagttgtaaaaaatgccactaaatcaaacgttaacaacgcagtaaatacattagtgg
aaagatggaatgaaaaatatgctcaagcttatccaaatgtaagtgcaaaaattgattatgatgacgaaatggcttacagtgaatcacaatta
attgcgaaatttggtacagcatttaaagctgtaaataatagcttgaatgtaaacttcggcgcaatcagtgaagggaaaatgcaagaagaa
gtcattagttttaaacaaatttactataacgtgaatgttaatgaacctacaagaccttccagatttttcggcaaagctgttactaaagagcagt
tgcaagcgcttggagtgaatgcagaaaatcctcctgcatatatctcaagtgtggcgtatggccgtcaagtttatttgaaattatcaactaatt
cccatagtactaaagtaaaagctgcttttgatgctgccgtaagcggaaaatctgtctcaggtgatgtagaactaacaaatatcatcaaaaat
tcttccttcaaagccgtaatttacggaggttccgcaaaagatgaagttcaaatcatcgacggcaacctcggagacttacgcgatattttga
aaaaaggcgctacttttaatcgagaaacaccaggagttcccattgcttatacaacaaacttcctaaaagacaatgaattagctgttattaaa
aacaactcagaatatattgaaacaacttcaaaagcttatacagatggaaaaattaacatcgatcactctggaggatacgttgctcaattcaa
catttcttgggatgaagtaaattatgatctcgagcatggagatacacctacattgcatgaatatatgttagatttgcaaccagagacaactg
atctctactgttatgagcaattaaatgacagctcagaggaggaggatgaaatagatggtccagctggacaagcagaaccggacagagc
ccattacaatattgtaaccttttgttgcaagtgtgactctacgcttcggttgtgcgtacaaagcacacacgtagacattcgtactttggaaga
cctgttaatgggcacactaggaattgtgtgccccatctgttctcagaaaccataaactagtctagtggtgatggtgatgatggagctcaga
tctgtctaagaggcagccatagggcataagctgtgtcaccagctgcaccgtggatgtcaggcagatgcccagaaggcgggagacata
tggggagcccacaccagccatcacgtatgcttcgtctaagatttctttgttggctttgggggatgtgttttccctcaacactttgatggccac
tggaattttcacattctccccatcagggatccagatgcccttgtagactgtgccaaaagcgccagatccaagcaccttcaccttcctcagc
tccgtctctttcaggatccgcatctgcgcctggttgggcatcgctccgctaggtgtcagcggctccaccagctccgtttcctgcagcagtc
tccgcatcgtgtacttccggatcttctgctgccctcgggcgcacagctggtggcaggccaggccctcgcccacacactcgtcctctggc
cggttggcagtgtggagcagagcttggtgcgggttccgaaagagctggtcccagggcaccgtgtgcacgaagcagaggtgggtgtt
atggtggatgagggccagtccactgcccagttccctcagtgagcgcagccccagccagctgatgcccagcccttgcagggtcagcga
gtaggcgccattgtgcagaattcgtccccggattacttgcaggttctggaagacgctgaggtcaggcaggctgtccggccatgctgag
atgtataggtaacctgtgatctcttccagagtctcaaacacttggagctgctctggctggagcggggcagtgttggaggctgggtcccca
tcaaagctctccggcagaaatgccaggctcccaaagatcttcttgcagccagcaaactcctggatattcttccacaaaatcgtgtcctggt
agcagagctgggggttccgctggatcaagacccctcctttcaagatctctgtgaggcttcgaagctgcagctcccgcaggcctcctgg
ggaggcccctgtgacaggggtggtattgttcagcgggtctccattgtctagcacggccagggcatagttgtcctcaaagagctgggtgc
ctcgcacaatccgcagcctctgcagtgggacctgcctcacttggttgtgagcgatgagcacgtagccctgcacctcctggatatcctgc
aggaaggacaggctggcattggtgggcaggtaggtgagttccaggtttccctgcaccacctggcagccctggtagaggtggcggag
catgtccaggtgggttctagatttatcacgtacccatttccccgcatcttttatttttttaaatactttagggaaaaatggtttttgatttgcttttaa
aggttgtggtgtagactcgtctgctgactgcatgctagaatctaagtcactttcagaagcatccacaactgactctttcgccacttttctctta
tttgcttttgttggtttatctggataagtaaggctttcaagctcactatccgacgacgctatggcttttcttctttttttaatttccgctgcgctatcc
gatgacagacctggatgacgacgctccacttgcagagttggtcggtcgactcctgaagcctcttcatttatagccacatttcctgtttgctc
accgttgttattattgttattcggacctttctctgcttttgctttcaacattgctattaggtctgctttgttcgtatttttcactttattcgatttttctagt
tcctcaatatcacgtgaacttacttcacgtgcagtttcgtatcttggtcccgtatttacctcgcttggctgctcttctgttttttcttcttcccattca
tctgtgtttagactggaatcttcgctatctgtcgctgcaaatattatgtcggggttaatcgtaatgcagttggcagtaatgaaaactaccatca
tcgcacgcataaatctgtttaatcccacttatactccctcctcgtgatacgctaatacaacctttttagaacaaggaaaattcggccttcatttt
cactaatttgttccgttaaaaattggattagcagttagttatcttcttaattagctaatataagaaaaaatattcatgaattattttaagaatatcac
ttggagaattaatttttctctaacatttgttaatcagttaaccccaactgcttcccaagcttcacccgggccactaactcaacgctagtagtgg
atttaatcccaaatgagccaacagaaccagaaccagaaacagaacaagtaacattggagttagaaatggaagaagaaaaaagcaatg
atttcgtgtgaataatgcacgaaatcattgcttatttttttaaaaagcgatatactagatataacgaaacaacgaactgaataaagaatacaa
aaaaagagccacgaccagttaaagcctgagaaactttaactgcgagccttaattgattaccaccaatcaattaaagaagtcgagaccca
aaatttggtaaagtatttaattactttattaatcagatacttaaatatctgtaaacccattatatcgggtttttgaggggatttcaagtctttaaga
agataccaggcaatcaattaagaaaaacttagttgattgccttttttgttgtgattcaactttgatcgtagcttctaactaattaattttcgtaaga
aaggagaacagctgaatgaatatcccttttgttgtagaaactgtgcttcatgacggcttgttaaagtacaaatttaaaaatagtaaaattcgc
tcaatcactaccaagccaggtaaaagtaaaggggctatttttgcgtatcgctcaaaaaaaagcatgattggcggacgtggcgttgttctg
acttccgaagaagcgattcacgaaaatcaagatacatttacgcattggacaccaaacgtttatcgttatggtacgtatgcagacgaaaac
cgttcatacactaaaggacattctgaaaacaatttaagacaaatcaataccttctttattgattttgatattcacacggaaaaagaaactatttc
agcaagcgatattttaacaacagctattgatttaggttttatgcctacgttaattatcaaatctgataaaggttatcaagcatattttgttttagaa
acgccagtctatgtgacttcaaaatcagaatttaaatctgtcaaagcagccaaaataatctcgcaaaatatccgagaatattttggaaagtc
tttgccagttgatctaacgtgcaatcattttgggattgctcgtataccaagaacggacaatgtagaattttttgatcccaattaccgttattcttt
caaagaatggcaagattggtctttcaaacaaacagataataagggctttactcgttcaagtctaacggttttaagcggtacagaaggcaa
aaaacaagtagatgaaccctggtttaatctcttattgcacgaaacgaaattttcaggagaaaagggtttagtagggcgcaatagcgttatg
tttaccctctctttagcctactttagttcaggctattcaatcgaaacgtgcgaatataatatgtttgagtttaataatcgattagatcaaccctta
gaagaaaaagaagtaatcaaaattgttagaagtgcctattcagaaaactatcaaggggctaatagggaatacattaccattctttgcaaag
cttgggtatcaagtgatttaaccagtaaagatttatttgtccgtcaagggtggtttaaattcaagaaaaaaagaagcgaacgtcaacgtgtt
catttgtcagaatggaaagaagatttaatggcttatattagcgaaaaaagcgatgtatacaagccttatttagcgacgaccaaaaaagaga
ttagagaagtgctaggcattcctgaacggacattagataaattgctgaaggtactgaaggcgaatcaggaaattttctttaagattaaacc
aggaagaaatggtggcattcaacttgctagtgttaaatcattgttgctatcgatcattaaattaaaaaaagaagaacgagaaagctatataa
aggcgctgacagcttcgtttaatttagaacgtacatttattcaagaaactctaaacaaattggcagaacgccccaaaacggacccacaac
tcgatttgtttagctacgatacaggctgaaaataaaacccgcactatgccattacatttatatctatgatacgtgtttgtttttctttgctggcta
gcttaattgcttatatttacctgcaataaaggatttcttacttccattatactcccattttccaaaaacatacggggaacacgggaacttattgt
acaggccacctcatagttaatggtttcgagccttcctgcaatctcatccatggaaatatattcatccccctgccggcctattaatgtgactttt
gtgcccggcggatattcctgatccagctccaccataaattggtccatgcaaattcggccggcaattttcaggcgttttcccttcacaaggat
gtcggtccctttcaattttcggagccagccgtccgcatagcctacaggcaccgtcccgatccatgtgtctttttccgctgtgtactcggctc
cgtagctgacgctctcgccttttctgatcagtttgacatgtgacagtgtcgaatgcagggtaaatgccggacgcagctgaaacggtatctc
gtccgacatgtcagcagacgggcgaaggccatacatgccgatgccgaatctgactgcattaaaaaagccttttttcagccggagtccag
cggcgctgttcgcgcagtggaccattagattctttaacggcagcggagcaatcagctctttaaagcgctcaaactgcattaagaaatagc
ctctttctttttcatccgctgtcgcaaaatgggtaaatacccctttgcactttaaacgagggttgcggtcaagaattgccatcacgttctgaac
ttcttcctctgtttttacaccaagtctgttcatccccgtatcgaccttcagatgaaaatgaagagaaccttttttcgtgtggcgggctgcctcct
gaagccattcaacagaataacctgttaaggtcacgtcatactcagcagcgattgccacatactccgggggaaccgcgccaagcaccaa
tataggcgccttcaatccctttttgcgcagtgaaatcgcttcatccaaaatggccacggccaagcatgaagcacctgcgtcaagagcag
cctttgctgtttctgcatcaccatgcccgtaggcgtttgctttcacaactgccatcaagtggacatgttcaccgatatgttttttcatattgctga
cattttcctttatcacggacaagtcaatttccgcccacgtatctctgtaaaaaggttttgtgctcatggaaaactcctctcttttttcagaaaatc
ccagtacgtaattaagtatttgagaattaattttatattgattaatactaagtttacccagttttcacctaaaaaacaaatgatgagataatagct
ccaaaggctaaagaggactataccaactatttgttaat.

In one embodiment, provided herein is a vaccine comprising a recombinant Listeria strain further comprising the recombinant nucleic acid encoding a first and at least a second polypeptide provided herein and an adjuvant, cytokine, chemokine, or a combination thereof.

In another embodiment, provided herein is a vaccine comprising a recombinant Listeria strain further comprising the recombinant nucleic acid encoding a first, at least a second polypeptide and a third polypeptide provided herein and an adjuvant, cytokine, chemokine, or a combination thereof.

In another embodiment, provided herein is a recombinant Listeria strain comprising an episomal recombinant nucleic acid molecule, the nucleic acid molecule comprising a first and at least a second open reading frame each encoding a first and at least a second polypeptide, wherein the first and the at least second polypeptide each comprise a heterologous antigen or a functional fragment thereof fused to an endogenous PEST-containing polypeptide.

In one embodiment, provided herein is a recombinant Listeria strain comprising a first integrated recombinant nucleic acid molecule comprising a first open reading frame encoding a polypeptide wherein the polypeptide comprises a heterologous antigenic or a functional fragment thereof, fused to an endogenous PEST-containing polypeptide, wherein the first nucleic acid molecule is integrated into the Listeria genome, and wherein the Listeria strain further comprises an episomal recombinant nucleic acid molecule, the episomal nucleic acid molecule comprising a first and at least a second open reading frame each encoding a first and at least a second polypeptide, wherein the first and the at least second polypeptide each comprise a heterologous antigen or a functional fragment thereof fused to an endogenous PEST-containing polypeptide.

In one embodiment, the first nucleic acid molecule provided herein that is to be integrated is a vector designed for site-specific homologous recombination into the Listeria genome. In another embodiment, the construct or heterologous gene is integrated into the Listerial chromosome using homologous recombination.

In one embodiment, a first nucleic acid molecule is operably integrated into the Listeria genome in an open reading frame with an endogenous nucleic acid sequence encoding an LLO, PEST or ActA sequence or functional fragments thereof while the at least second nucleic acid molecules is expressed from an episomal vector, each with an endogenous nucleic acid sequence encoding an LLO, PEST or ActA sequence or functional fragments thereof. In one embodiment, the integration does not eliminate the functionality of LLO. In another embodiment, the integration does not eliminate the functionality of ActA. In another embodiment, the functionality of LLO or ActA is its native functionality. In another embodiment, the LLO functionality is allowing the organism to escape from the phagolysosome, while in another embodiment, the LLO functionality is enhancing the immunogenicity of a polypeptide to which it is fused. In one embodiment, a recombinant Listeria of the present invention retains genomic LLO function, which in another embodiment, is hemolytic function and in another embodiment, is antigenic function. Other functions of LLO are known in the art, as are methods of and assays for evaluating LLO functionality. In one embodiment, a recombinant Listeria of the present invention has wild-type virulence, while in another embodiment, a recombinant Listeria of the present invention has attenuated virulence. In another embodiment, a recombinant Listeria of the present invention is avirulent. In one embodiment, a recombinant Listeria of the present invention expresses a fused antigen-truncated LLO fusion protein. Thus, in one embodiment, the integration of the first nucleic acid molecule into the Listeria genome does not disrupt the structure of the endogenous PEST-containing polypeptide, while in another embodiment, it does not disrupt the function of the endogenous PEST-containing polypeptide. In one embodiment, the integration of a nucleic acid molecule into the Listeria genome does not disrupt the ability of the Listeria to express native LLO. In one embodiment, the integration of a first nucleic acid molecule into the Listeria genome does not disrupt the ability of the Listeria to escape the phagolysosome.

In another embodiment, the present invention provides a recombinant Listeria strain comprising at least one episomal recombinant nucleic acid molecule, the nucleic acid molecules comprising a first and at least a second open reading frame each encoding a first and at least a second polypeptide, wherein the first and the at least second polypeptide each comprise a heterologous antigen or a functional fragment thereof fused to an endogenous PEST-containing polypeptide, wherein the nucleic acids further comprise an open reading frame encoding a plasmid replication control region. In another embodiment, the plasmid control region regulates replication of the episomal recombinant nucleic acid molecule.

In another embodiment, the plasmid control region comprises an open reading frame encoding a transcription repressor that represses heterologous antigen expression from the first or at least second nucleic acid molecule. In another embodiment, the plasmid control region comprises an open reading frame encoding transcription inducer that induces heterologous antigen expression from the first or at least second nucleic acid molecule. In another embodiment, the plasmid control region comprises an open reading frame encoding a transcription repressor that represses heterologous antigen expression from the first, second or third nucleic acid molecule. In another embodiment, the plasmid control region comprises an open reading frame encoding a transcription inducer that induces heterologous antigen expression from the first, second or third nucleic acid molecule.

In another embodiment, the plasmid replication regulation region enables the regulation of expression of exogenous heterologous antigen from each of the first or the at least second nucleic acid molecule. In another embodiment, the plasmid replication regulation region enables the regulation of expression of exogenous heterologous antigen from each of the first, second or third nucleic acid molecules.

In one embodiment, measuring metabolic burden is accomplished by any means know in the art at the time of the invention which include but are not limited to, measuring growth rates of the vaccine strain, optical density readings, colony forming unit (CFU) plating, and the like. In another embodiment, the metabolic burden on the bacterial cell is determined by measuring the viability of the bacterial cell. Methods of measuring bacteria viability are readily known and available in the art, some of which include but are not limited to, bacteria plating for viability count, measuring ATP, and flow cytometry. In ATP staining, detection is based on using the luciferase reaction to measure the amount of ATP from viable cells, wherein the amount of ATP in cells correlates with cell viability. As to flow cytometry, this method can be used in various ways, also known in the art, for example after employing the use of viability dyes which are excluded by live bacterial cells and are absorbed or adsorbed by a dead bacterial cells. A skilled artisan would readily understand that these and any other methods known in the art for measuring bacterial viability can be used in the present invention. It is to be understood that a skilled artisan would be able to implement the knowledge available in the art at the time of the invention for measuring growth rates of the vaccine strain or expression of marker genes by the vaccine strain that enable determining the metabolic burden of the vaccine strain expressing multiple heterologous antigens or functional fragments thereof.

In another embodiment, a recombinant Listeria strain comprising an episomal recombinant nucleic acid molecule, the nucleic acid molecule comprising a first and at least a second open reading frame each encoding a first and at least a second polypeptide, wherein the first and the at least second polypeptide each comprise a heterologous antigen or a functional fragment thereof fused to an endogenous PEST-containing polypeptide, wherein the nucleic acid further comprises an open reading frame encoding a plasmid replication control region.

In one embodiment, the present invention provides a method of producing a recombinant Listeria strain comprising an episomal expression plasmid comprising a first and at least a second nucleic acid encoding a first and at least a second polypeptide, wherein the first and the second polypeptide each comprise a heterologous antigen fused to an endogenous PEST-containing polypeptide, the method comprising the steps of a) recombinantly fusing in the plasmid the first and the second nucleic acid encoding the first and the second polypeptide each comprising a first and a second heterologous antigen fused to an endogenous PEST-containing polypeptide; b) transforming the recombinant Listeria with the episomal expression plasmid; and, c) expressing the first, and the at least second antigens under conditions conducive to antigenic expression in the recombinant Listeria strain.

In one embodiment, provided herein is a method of producing a recombinant Listeria strain comprising an episomal expression plasmid comprising a first, a second and a third nucleic acid encoding a first a second and a third polypeptide, wherein the first, the second and the third polypeptide each comprise a heterologous antigen fused to an endogenous PEST-containing polypeptide, the method comprising the steps of: a) recombinantly fusing in the plasmid the first, the second and the third nucleic acid encoding the first, the second and the third polypeptide each comprising a first, a second and a third heterologous antigen fused to an endogenous PEST-containing polypeptide; b) transforming the recombinant Listeria with the episomal expression plasmid; and, c) expressing the first, the second and the third antigens under conditions conducive to antigenic expression in the recombinant Listeria strain.

In one embodiment, provided herein is a method of producing a recombinant Listeria strain comprising an integrated first nucleic acid, and an episomal expression plasmid comprising a second, and a third nucleic acid each encoding a first, a second, and a third polypeptide, wherein the first, second and third polypeptides each comprise a heterologous antigen fused to an endogenous PEST-containing polypeptide, the method comprising the steps of a) integrating the first nucleic acid encoding the first polypeptide comprising a first heterologous antigen fused to an endogenous PEST-containing polypeptide into the recombinant Listeria's genome; b) recombinantly fusing in the plasmid the second and the third nucleic acid encoding the second and the third polypeptide each comprising a second and a third heterologous antigen fused to an endogenous PEST-containing polypeptide; c) transforming the recombinant Listeria with the episomal expression plasmid; and, d) expressing the first, second, and third antigens under conditions conducive to antigenic expression in the recombinant Listeria strain.

In one embodiment, provided herein is a method of producing a recombinant Listeria strain comprising at least one episomal expression plasmid comprising a first and at least a second nucleic acid encoding a first and at least a second polypeptide, wherein the first and the at least second polypeptide each comprise a heterologous antigen fused to an endogenous PEST-containing polypeptide, the method comprising the steps of a) recombinantly fusing in each plasmid the first and the at least second nucleic acid encoding the first and the second polypeptide each comprising a first and a second heterologous antigen fused to an endogenous PEST-containing polypeptide; b) transforming the recombinant Listeria with each of the episomal expression plasmid; and, c) expressing the first, and the at least second antigens under conditions conducive to antigenic expression in the recombinant Listeria strain, and wherein if the expression of the first, and the at least second antigens place a metabolic burden on the Listeria, each of the plasmids' replication control region activates and expresses a repressor that represses plasmid replication and represses expression of the first, second, and the third heterologous antigen or fragment thereof from each plasmid.

In one embodiment, provided herein a method of producing a recombinant Listeria strain comprising an episomal expression plasmid comprising a plasmid replication control region, and a first, second, and third nucleic acid encoding a first, second and third polypeptide, wherein the first, second and third polypeptide comprise a heterologous antigen fused to an endogenous PEST-containing polypeptide, the method comprising the steps of a) recombinantly fusing in the plasmid the first, second and third nucleic acid encoding the first, second and third polypeptide comprising a first, second and third heterologous antigen fused to an endogenous PEST-containing; b) transforming the recombinant Listeria with the episomal expression plasmid; and, c) expressing the first, the second, and the third antigens under conditions conducive to antigenic expression in the recombinant Listeria strain, and wherein if the expression of the first, the second, and the third antigens place a metabolic burden on the Listeria, the plasmid's replication control region activates and expresses a repressor that represses replication from the plasmid and expression of the first, second, and the third heterologous antigen or fragment thereof.

In one embodiment, the recombinant Listeria provided herein comprises up to four episomal recombinant nucleic acid molecules, each comprising a first and at least a second open reading frame, wherein each of said first and at least second open reading frame encode a first polypeptide and at least a second polypeptide, wherein said first and said at least second polypeptide each comprise a heterologous antigen or a functional fragment thereof fused to an endogenous PEST-containing polypeptide, and wherein each of said recombinant nucleic acid further comprise an open reading frame encoding said plasmid replication control region. In another embodiment, the recombinant Listeria provided herein comprises up to five episomal recombinant nucleic acid molecules. In another embodiment, each of the plasmid replication control regions regulate the expression of each episomal recombinant nucleic acid copy number to 3 or 4 copies per Listeria.

In one embodiment, provided herein is a method of producing at least one recombinant Listeria strain comprising an episomal expression plasmid comprising a first, second, and third nucleic acid encoding a first, second and third polypeptide, wherein the first, second and third polypeptide comprise a heterologous antigen fused to an endogenous PEST-containing polypeptide, the method comprising the steps of a) recombinantly fusing in each of the plasmids the first, second and third nucleic acid encoding the first, second and third polypeptide comprising a first, second and third heterologous antigen fused to an endogenous PEST-containing; b) transforming the recombinant Listeria with each of the episomal expression plasmids; and, c) expressing the first, the second, and the third antigens under conditions conducive to antigenic expression in the recombinant Listeria strain, and wherein if the expression of the first, the second, and the third antigens from each plasmid place a metabolic burden on the Listeria, each of the plasmids' replication control region activates and expresses a repressor that represses plasmid replication and represses expression of the first, second, and the third heterologous antigen or fragment thereof from each plasmid.

In one embodiment, provided herein is a plasmid or recombinant nucleic acid library comprising the monovalent or bivalent plasmids or the episomal recombinant nucleic acids of the present invention that can be combined in as appropriate to any given subject's gene expression pattern. In another embodiment, each bivelent plasmid or episomal recombinant nucleic acid from the library encodes at least two distinct heterologous antigen/PEST-containing polypeptides fusion proteins. It is to be understood that a library of validated plasmids can be created through any means well known in the art and maintained and then used as parts for the creation of a bivalent plasmid uniquely suited to a given subject's gene expression profile. In another embodiment, such plasmids could be used with a single genomically inserted fusion protein. Such libraries can be the source of populations of combinatorial molecules that can be further manipulated or analyzed, for example, by protein expression and screening for fusion proteins having desirable characteristics.

In one embodiment, the recombinant nucleic acid library is a cDNA library, an mRNA library, a plasmid library, etc.

In one embodiment, the heterologous antigen or functional fragments thereof and the endogenous PEST-containing polypeptide provided herein are translated in a single open reading frame. In another embodiment each heterologous antigenic polypeptides and the endogenous PEST-containing polypeptide provided herein are fused after being translated separately.

In another embodiment, the nucleic acid sequences of methods and compositions provided herein are operably linked to a promoter/regulatory sequence. In another embodiment, each of the nucleic acid sequences is operably linked to a promoter/regulatory sequence. In one embodiment, the promoter/regulatory sequence is present on an episomal plasmid comprising the nucleic acid sequence. In one embodiment, endogenous Listeria promoter/regulatory sequences control the expression of a nucleic acid sequence of the methods and compositions of the present invention. Each possibility represents a separate embodiment of the methods and compositions provided herein.

In another embodiment, a nucleic acid sequence provided herein is operably linked to a promoter, regulatory sequence, or combination thereof that drives expression of the encoded peptide in the Listeria strain. Promoter, regulatory sequences, and combinations thereof useful for driving constitutive expression of a gene are well known in the art and include, but are not limited to, for example, the P_{hlyA}, P_{ActA}, hly, ActA, and p60 promoters of Listeria, the Streptococcus bac promoter, the Streptomyces griseus sgiA promoter, and the B. thuringiensis phaZ promoter. In another embodiment, inducible and tissue specific expression of the nucleic acid encoding a peptide provided herein is accomplished by placing the nucleic acid encoding the peptide under the control of an inducible or tissue-specific promoter/regulatory sequence. Examples of tissue-specific or inducible regulatory sequences, promoters, and combinations thereof which are useful for his purpose include, but are not limited to the MMTV LTR inducible promoter, and the SV40 late enhancer/promoter. In another embodiment, a promoter that is induced in response to inducing agents such as metals, glucocorticoids, and the like, is utilized. Thus, it will be appreciated that the invention includes the use of any promoter or regulatory sequence, which is either known or unknown, and which is capable of driving expression of the desired protein operably linked thereto. In one embodiment, a regulatory sequence is a promoter, while in another embodiment, a regulatory sequence is an enhancer, while in another embodiment, a regulatory sequence is a suppressor, while in another embodiment, a regulatory sequence is a repressor, while in another embodiment, a regulatory sequence is a silencer.

In another embodiment, the present invention provides an isolated nucleic acid encoding a recombinant polypeptide. In one embodiment, the isolated nucleic acid comprises a sequence sharing at least 80% homology with a nucleic acid encoding a recombinant polypeptide provided herein. In one embodiment, the isolated nucleic acid comprises a sequence sharing at least 85% homology with a nucleic acid encoding a recombinant polypeptide provided herein. In another embodiment, the isolated nucleic acid comprises a sequence sharing at least 90% homology with a nucleic acid encoding a recombinant polypeptide provided herein. In another embodiment, the isolated nucleic acid comprises a sequence sharing at least 95% homology with a nucleic acid encoding a recombinant polypeptide provided herein. In another embodiment, the isolated nucleic acid comprises a sequence sharing at least 97% homology with a nucleic acid encoding a recombinant polypeptide provided herein. In another embodiment, the isolated nucleic acid comprises a sequence sharing at least 99% homology with a nucleic acid encoding a recombinant polypeptide provided herein.

In one embodiment, the recombinant Listeria expresses at least two or more distinct heterologous antigens. In another embodiment, the recombinant Listeria expresses at least three or more distinct heterologous antigens. In another embodiment, the recombinant Listeria expresses at least four or more distinct heterologous antigens. In another embodiment, provided herein is a method of producing a recombinant Listeria strain expressing three distinct heterologous antigens. In another embodiment, expression of the distinct heterologous antigens is from the episomal vector comprised within the recombinant Listeria strain. In another embodiment, expression of at least two distinct heterologous antigens is from an episomal recombinant nucleic acid in the Listeria. In another embodiment, the Listeria is a recombinant Listeria monocytogenes strain.

In one embodiment, an endogenous open reading frame encoding endogenous polypeptide comprising a PEST-containing polypeptide provided herein is a truncated, non-hemolytic LLO, an N-terminal truncated ActA, a PEST sequence, or functional fragments of each.

In one embodiment, the method provided herein comprises transforming the recombinant Listeria with an episomal recombinant nucleic acid comprising at least two open reading frames encoding at least two polypeptides comprising at least two distinct heterologous antigens. In another embodiment, the method provided herein comprises transforming the recombinant Listeria with an episomal recombinant nucleic acid comprising at least two open reading frames encoding at least two polypeptides comprising at least two distinct heterologous antigens and with an integrating vector comprising one nucleic acid encoding an additional heterologous antigen. In another embodiment, the method comprises transforming said recombinant Listeria with an episomal recombinant nucleic acid encoding at least three distinct heterologous antigens.

In yet another embodiment, the method comprises expressing the first and at least second antigens under conditions conducive to antigenic expression, that are known in the art, in the recombinant Listeria strain. In yet another embodiment, the method comprises expressing the first, second and third antigens under conditions conducive to antigenic expression that are known in the art, in the recombinant Listeria strain.

In one embodiment, the recombinant Listeria strain expresses more than two antigens, which are expressed from one recombinant episomal nucleic acid molecules in the Listeria. In another embodiment, the recombinant Listeria strain expresses more than three antigens, which are expressed from one recombinant episomal nucleic acid molecules and one integrated nucleic acid in the Listeria. Thus, as described hereinabove, in one embodiment, a recombinant Listeria strain provided herein comprises two or more antigens. In another embodiment, each of the antigens are expressed as a fusion protein with LLO, which in one embodiment, is non-hemolytic LLO, and, in another embodiment, truncated LLO. In another embodiment, each of the antigens is expressed as a fusion protein with ActA, which in one embodiment is truncated ActA. In another embodiment, each of the antigens is expressed as a fusion protein with PEST. In one embodiment, a recombinant Listeria strain provided herein targets tumors by eliciting immune responses to at least two separate antigens, which are expressed by two different tumor cell types. In one embodiment, the recombinant Listeria strain provided herein targets tumors by eliciting an immune response to at least two different antigens expressed by the same cell type. In another embodiment, the at least two heterologous antigens are a cell surface antigen and an anti-angiogenic antigen. In another embodiment, a recombinant Listeria strain provided herein targets tumors by eliciting an immune response to at least two different antigens as described herein below or as are known in the art. In another embodiment, a recombinant Listeria strain provided herein targets tumors by eliciting an immune response to at least three different antigens provided herein or as are known in the art.

In one embodiment, the first, or the at least second polypeptide provided herein comprises an antigen associated with the local tissue environment that is further associated with the development or metastasis of cancer. In another embodiment, the first, or at least second polypeptide comprises an antigen associated with tumor immune evasion or resistance to cancer.

In one embodiment, the antigens provided herein can be selected from but are not limited to prostate specific antigen (PSA) and prostate-specific membrane antigen (PSMA), which in one embodiment is FOLH1, HPV-E7, HPV-E6, SCCE, NY-ESO-1, PSMA, prostate stem cell antigen (PSCA), WT-1, HIV-1 Gag, CEA, LMP-1, p53, Proteinase 3, Tyrosinase related protein 2, Muc1 EGFR-III, VEGF-R or any other cancer-associated antigen or any other antigen associated with tumor immune evasion or resistance to cancer. In another embodiment, the antigen is HMW-MAA or a functional fragment thereof.

In one embodiment, a first antigen of the compositions and methods of the present invention is directed against a specific cell surface antigen or tumor target, and at least a second antigen is directed against an angiogenic antigen or tumor microenvironment. In one embodiment, a first antigen of the compositions and methods of the present invention is directed against an angiogenic antigen or tumor microenvironment, and at least a second antigen is directed against a specific cell surface antigen. In another embodiment, the first and at least second antigen of the compositions and methods of the present invention are polypeptides expressed by tumor cells, or in another embodiment, polypeptides expressed in a tumor microenvironment. In another embodiment, the first antigen of the compositions and methods of the present invention is a polypeptide expressed by a tumor and at least the second antigen of the compositions and methods of the present invention is a receptor target, including but not limited to, NO Synthetase, Arg-1, or other enzyme known in the art.

In another embodiment, provided herein is a method of inhibiting the onset of cancer, the method comprising the step of administering a recombinant Listeria composition that expresses at least two distinct heterologous antigens specifically expressed in the cancer from an episomal recombinant nucleic acid or plasmid.

In one embodiment, provided herein is a method of treating a first and at least a second tumor in a subject, the method comprising the step of administering a recombinant Listeria composition that expresses at least two distinct heterologous antigens specifically expressed on the first and at least second tumor, from an episomal recombinant nucleic acid or plasmid.

In another embodiment, provided herein is a method of ameliorating symptoms that are associated with a cancer in a subject, the method comprising the step of administering a recombinant Listeria composition that expresses at least two distinct heterologous antigens specifically expressed in the cancer from an episomal recombinant nucleic acid or plasmid.

In one embodiment, provided herein is a method of protecting a subject from cancer, the method comprising the step of administering a recombinant Listeria composition that expresses at least two distinct heterologous antigens specifically expressed in the cancer from an episomal recombinant nucleic acid or plasmid.

In another embodiment, provided herein is a method of delaying onset of cancer, the method comprising the step of administering a recombinant Listeria composition that expresses at least three distinct heterologous antigens specifically expressed in the cancer. In another embodiment, provided herein is a method of treating metastatic cancer, the method comprising the step of administering a recombinant Listeria composition that expresses two distinct heterologous antigens specifically expressed in the cancer. In another embodiment, provided herein is a method of preventing metastatic cancer or micrometastatis, the method comprising the step of administering a recombinant Listeria composition that expresses two distinct heterologous antigens specifically expressed in the cancer. In another embodiment, the recombinant Listeria composition is administered orally or parenterally.

In one embodiment, provided herein is a method of inducing an immune response to at least two antigens in a subject comprising administering a recombinant Listeria strain of the present invention to the subject. In one embodiment, provided herein is a method of inducing an anti-angiogenic immune response to at least two antigens in a subject, comprising administering a recombinant Listeria strain provided herein to the subject. In another embodiment, the recombinant Listeria strain comprises an episomal recombinant nucleic acid comprising a first and at least a second open reading frame encoding a first and at least a second polypeptide comprising a first and at least a second antigen fused to a PEST-containing sequence. In yet another embodiment, the first nucleic acid molecule is operably integrated into the Listeria genome as an open reading frame with an endogenous polypeptide comprising a PEST sequence, and the second and third episomal recombinant nucleic acids each encode a second and third polypeptide comprising a heterologous antigen fused to a PEST-containing polypeptide.

In one embodiment, provided herein is a method of treating, suppressing, or inhibiting at least one tumor or cancer in a subject comprising administering a recombinant Listeria strain provided herein to the subject. In another embodiment, the tumor is a prostate tumor, brain tumor, lung tumor, gastrointestinal tumor, pancreatic tumor, an ovarian tumor, breast tumor, or a combination thereof. In another embodiment, the tumor is a cancer, in yet another embodiment, the cancer is a metastatic cancer. In another embodiment, the cancer is a prostate cancer, brain cancer, lung cancer, gastrointestinal cancer, pancreatic cancer, an ovarian cancer, breast cancer, or a combination thereof.

In one embodiment, provided herein is a method of delaying the onset of a cancer in a subject comprising administering a recombinant Listeria strain provided herein to the subject. In another embodiment, provided herein is a method of delaying the progression to a cancer in a subject comprising administering a recombinant Listeria strain provided herein to the subject. In another embodiment, provided herein is a method of extending the remission to a cancer in a subject comprising administering a recombinant Listeria strain provided herein to the subject. In another embodiment, provided herein is a method of decreasing the size of an existing tumor in a subject comprising administering a recombinant Listeria strain provided herein to the subject. In another embodiment, provided herein is a method of preventing the growth of an existing tumor in a subject comprising administering a recombinant Listeria strain provided herein to the subject. In another embodiment, provided herein is a method of preventing the growth of new or additional tumors in a subject comprising administering a recombinant Listeria strain provided herein to the subject.

In another embodiment, the present invention provides a method of impeding angiogenesis of a solid tumor in a subject, comprising administering to the subject a composition comprising a recombinant Listeria provided herein. In another embodiment, an antigen of the invention is HMW-MAA. In another embodiment, the antigen is fibroblast growth factor (FGF). In another embodiment, an antigen of the invention is vascular endothelial growth factor (VEGF). In another embodiment, the antigen is any other antigen known in the art to be involved in angiogenesis. In another embodiment, the methods and compositions of impeding angiogenesis of a solid tumor in a subject, provided herein, comprise administering to the subject a composition comprising a recombinant Listeria encoding at least three heterologous antigens, provided herein. In another embodiment, one of the three heterologous antigens is HMW-MAA. In another embodiment, the antigen is any other antigen known in the art to be involved in angiogenesis. Each possibility represents a separate embodiment of the methods and compositions provided herein.

In another embodiment, an episomal expression vector of the methods and compositions provided herein comprises at least two or more heterologous antigens fused in frame to a nucleic acid sequence encoding a PEST-like AA sequence. In another embodiment, the PEST-like AA sequence is KENSISSMAPPASPPASPKTPIEKKHADEIDK (SEQ ID NO: 1). In another embodiment, the PEST sequence is KENSISSMAPPASPPASPK (SEQ ID No: 2). In another embodiment, fusion of an antigen to any LLO sequence that includes one of the PEST-like AA sequences enumerated herein can enhance cell mediated immunity against HMW-MAA.

In another embodiment, the PEST-like AA sequence is a PEST sequence from a Listeria ActA protein. In another embodiment, the PEST sequence is KTEEQPSEVNTGPR (SEQ ID NO: 3), KASVTDTSEGDLDSSMQSADESTPQPLK (SEQ ID NO: 4), KNEEVNASDFPPPPTDEELR (SEQ ID NO: 5), or RGGIPTSEEFSSLNSGDFTDDENSETTEEEIDR (SEQ ID NO: 6). In another embodiment, the PEST sequence is from Listeria seeligeri cytolysin, encoded by the lso gene. In another embodiment, the PEST sequence is RSEVTISPAETPESPPATP (SEQ ID NO: 7). In another embodiment, the PEST sequence is from Streptolysin O protein of Streptococcus sp. In another embodiment, the PEST sequence is from Streptococcus pyogenes Streptolysin O, e.g. KQNTASTETTTTNEQPK (SEQ ID NO: 8) at AA 35-51. In another embodiment, the PEST sequence is from Streptococcus equisimilis Streptolysin O, e.g. KQNTANTETTTTNEQPK (SEQ ID NO: 9) at AA 38-54. In another embodiment, the PEST sequence has a sequence selected from SEQ ID NO: 3-9. In another embodiment, the PEST sequence has a sequence selected from SEQ ID NO: 1-9. In another embodiment, the PEST sequence is another PEST-like AA sequence derived from a prokaryotic organism.

Identification of PEST sequences is well known in the art, and is described, for example in Rogers S et al (Amino acid sequences common to rapidly degraded proteins: the PEST hypothesis. Science 1986; 234(4774):364-8, incorporated herein by reference) and Rechsteiner M et al (PEST sequences and regulation by proteolysis. Trends Biochem Sci 1996; 21(7):267-71, incorporated herein by reference). “PEST sequence” refers, in another embodiment, to a region rich in proline (P), glutamic acid (E), serine (S), and threonine (T) residues. In another embodiment, the PEST sequence is flanked by one or more clusters containing several positively charged amino acids. In another embodiment, the PEST sequence mediates rapid intracellular degradation of proteins containing it. In another embodiment, the PEST sequence fits an algorithm disclosed in Rogers et al. In another embodiment, the PEST sequence fits an algorithm disclosed in Rechsteiner et al. In another embodiment, the PEST sequence contains one or more internal phosphorylation sites, and phosphorylation at these sites precedes protein degradation. In one embodiment, a sequence referred to herein as a PEST-like sequence is a PEST sequence.

In one embodiment, PEST sequences of prokaryotic organisms are identified in accordance with methods such as described by, for example Rechsteiner and Rogers (1996, Trends Biochem. Sci. 21:267-271) for LM and in Rogers S et al (Science 1986; 234(4774):364-8). Alternatively, PEST-like AA sequences from other prokaryotic organisms can also be identified based on this method. Other prokaryotic organisms wherein PEST-like AA sequences would be expected to include, but are not limited to, other Listeria species. In one embodiment, the PEST sequence fits an algorithm disclosed in Rogers et al. In another embodiment, the PEST sequence fits an algorithm disclosed in Rechsteiner et al. In another embodiment, the PEST sequence is identified using the PEST-find program.

Each method for identifying a PEST sequence represents a separate embodiment provided herein.

In another embodiment, the PEST sequence is any other PEST sequence known in the art. Each PEST sequence and type thereof represents a separate embodiment provided herein.

In one embodiment, “fusion” refers to two peptides or protein fragments either linked together at their respective ends or embedded one within the other. In another embodiment the linkage is a covalent linkage. Each possibility represents a separate embodiment of the methods and compositions provided herein.

In another embodiment, a recombinant Listeria strain of the compositions and methods provided herein comprises a full length LLO polypeptide, which in one embodiment, is hemolytic.

In another embodiment, an LLO protein fragment is utilized in compositions and methods provided herein. In one embodiment, a truncated LLO protein is encoded by the episomal expression vector provided herein that expresses a polypeptide, that is, in one embodiment, an antigen, in another embodiment, an angiogenic factor, or, in another embodiment, both an antigen and angiogenic factor. In another embodiment, the LLO fragment is an N-terminal fragment.

In another embodiment, the N-terminal LLO fragment has the sequence:

MKKIMLVFITLILVSLPIAQQTEAKDASAFNKENSISSVAPPASPPASPKTPIEK KHADEIDKYIQGLDYNKNNVLVYHGDAVTNVPPRKGYKDGNEYIVVEKKKKSINQN NADIQVVNAISSLTYPGALVKANSELVENQPDVLPVKRDSLTLSIDLPGMTNQDNKIV VKNATKSNVNNAVNTLVERNEKYAQAYSNVSAKIDYDDEMAYSESQLIAKFGTA FKAVNNSLNVNFGAISEGKMQEEVISFKQIYYNVNVNEPTRPSRFFGKAVTKEQLQA LGVNAENPPAYISSVAYGRQVYLKLSTNSHSTKVKAAFDAAVSGKSVSGDVELTNII KNSSFKAVIYGGSAKDEVQIIDGNLGDLRDILKKGATFNRETPGVPIAYTTNFLKDNE LAVIKNNSEYIETTSKAYTDGKINIDHSGGYVAQFNISWDEVNYD (SEQ ID NO: 10). In another embodiment, an LLO AA sequence of methods and compositions provided herein comprises the sequence set forth in SEQ ID No: 10. In another embodiment, the LLO AA sequence is a homologue of SEQ ID No: 10. In another embodiment, the LLO AA sequence is a variant of SEQ ID No: 10. In another embodiment, the LLO AA sequence is a fragment of SEQ ID No: 10. In another embodiment, the LLO AA sequence is an isoform of SEQ ID No: 10. Each possibility represents a separate embodiment of the methods and compositions provided herein.

In another embodiment, the LLO fragment has the sequence:

mkkmilvfitlilvslpiaqqteakdasafnkensissvappasppaspktpiekkhadeidkyiqgldynknnylvy hgdavtnvpprkgykdgneyivvelkkksinqnnadiqvvnaissltypgalvkanselvenqpdvlpvkrdsldsidlpgmtn qdnkivvknatksnvnnavntiverwnekyaqaysnvsakidyddemaysesqliakfgtafkavnnslnvnfgaisegkmqe evisfkqiyynvnvneptrpsrffgkavtkeqlqalgvnaenppayissvaygrqvylklstnshstkvkaafdaaysgksvsgdv eltniiknssfkaviyggsakdevqiidgnlgdlrdilkkgatfnretpgvpiayttnflkdnelaviknnseyiettskaytd (SEQ ID NO: 11). In another embodiment, an LLO AA sequence of methods and compositions provided herein comprises the sequence set forth in SEQ ID No: 11. In another embodiment, the LLO AA sequence is a homologue of SEQ ID No: 11. In another embodiment, the LLO AA sequence is a variant of SEQ ID No: 11. In another embodiment, the LLO AA sequence is a fragment of SEQ ID No: 11. In another embodiment, the LLO AA sequence is an isoform of SEQ ID No: 11. Each possibility represents a separate embodiment of the methods and compositions provided herein.

The LLO protein used in the compositions and methods provided herein has, in another embodiment, the sequence set forth in GenBank Accession No. P13128 or GenBank Accession No. X15127. The first 25 AA of the proprotein corresponding to this sequence are the signal sequence and are cleaved from LLO when it is secreted by the bacterium. Thus, in this embodiment, the full length active LLO protein is 504 residues long. In another embodiment, the above LLO fragment is used as the source of the LLO fragment incorporated in a vaccine provided herein. In another embodiment, an LLO AA sequence of methods and compositions provided herein comprises the sequence set forth in GenBank Accession No. P13128 or GenBank Accession No. X15127. In another embodiment, the LLO AA sequence is a homologue of GenBank Accession No. P13128 or GenBank Accession No. X15127. In another embodiment, the LLO AA sequence is a variant of GenBank Accession No. P13128 or GenBank Accession No. X15127. In another embodiment, the LLO AA sequence is a fragment of GenBank Accession No. P13128 or GenBank Accession No. X15127. In another embodiment, the LLO AA sequence is an isoform of GenBank Accession No. P13128 or GenBank Accession No. X15127. Each possibility represents a separate embodiment provided herein.

The LLO protein used in the compositions and methods provided herein has, in another embodiment, the sequence:

MKKIMLVFITLILVSLPIAQQTEAKDASAFNKENSISSVAPPASPPASPKTPIEKKHADE IDKYIQGLDYNKNNVLVYHGDAVTNVPPRKGYKDGNEYIVVEKKKKSINQNNADIQ VVNAISSLTYPGALVKANSELVENQPDVLPVKRDSLTLSIDLPGMTNQDNKIVVKNA TKSNVNNAVNTLVERNEKYAQAYSNVSAKIDYDDEMAYSESQLIAKFGTAFKAV NNSLNVNFGAISEGKMQEEVISFKQIYYNVNVNEPTRPSRFFGKAVTKEQLQALGVN AENPPAYISSVAYGRQVYLKLSTNSHSTKVKAAFDAAVSGKSVSGDVELTNIIKNSSF KAVIYGGSAKDEVQIIDGNLGDLRDILKKGATFNRETPGVPIAYTTNFLKDNELAVIK NNSEYIETTSKAYTD (SEQ ID NO: 12). In another embodiment, an LLO AA sequence of methods and compositions provided herein comprises the sequence set forth in SEQ ID NO: 12. In another embodiment, the LLO AA sequence is a homologue of SEQ ID NO: 12. In another embodiment, the LLO AA sequence is a variant of SEQ ID NO: 12. In another embodiment, the LLO AA sequence is a fragment of SEQ ID NO: 12. In another embodiment, the LLO AA sequence is an isoform of SEQ ID NO: 12. Each possibility represents a separate embodiment provided herein.

In one embodiment, the amino acid sequence of the LLO polypeptide of the compositions and methods provided herein is from the Listeria monocytogenes 10403S strain, as set forth in Genbank Accession No.: ZP_{—}01942330, EBA21833, or is encoded by the nucleic acid sequence as set forth in Genbank Accession No.: NZ_AARZ01000015 or AARZ01000015.1. In another embodiment, the LLO sequence for use in the compositions and methods provided herein is from Listeria monocytogenes, which in one embodiment, is the 4b F2365 strain (in one embodiment, Genbank accession number: YP_{—}012823), the EGD-e strain (in one embodiment, Genbank accession number: NP_{—}463733), or any other strain of Listeria monocytogenes known in the art.

Each LLO protein and LLO fragment represents a separate embodiment of the methods and compositions provided herein.

In another embodiment, homologues of LLO from other species, including known lysins, or fragments thereof may be used to create the fusion proteins of LLO and an antigen of the compositions and methods provided herein, which in one embodiment, is HMW-MAA, and in another embodiment is a fragment of HMW-MAA.

In another embodiment, the LLO fragment of methods and compositions provided herein, is a PEST-containing polypeptide and is utilized as part of a composition or in the methods provided herein.

In another embodiment, the LLO fragment consists of about the first 441 AA of the LLO protein. In another embodiment, the LLO fragment comprises about the first 400-441 AA of the 529 AA full length LLO protein. In another embodiment, the LLO fragment corresponds to AA 1-441 of an LLO protein disclosed herein. In another embodiment, the LLO fragment consists of about the first 420 AA of LLO. In another embodiment, the LLO fragment corresponds to AA 1-420 of an LLO protein disclosed herein. In another embodiment, the LLO fragment consists of about AA 20-442 of LLO. In another embodiment, the LLO fragment corresponds to AA 20-442 of an LLO protein disclosed herein. In another embodiment, any ALLO without the activation domain comprising cysteine 484, and in particular without cysteine 484, are suitable for methods and compositions provided herein.

In another embodiment, the LLO fragment corresponds to the first 400 AA of an LLO protein. In another embodiment, the LLO fragment corresponds to the first 300 AA of an LLO protein. In another embodiment, the LLO fragment corresponds to the first 200 AA of an LLO protein. In another embodiment, the LLO fragment corresponds to the first 100 AA of an LLO protein. In another embodiment, the LLO fragment corresponds to the first 50 AA of an LLO protein, which in one embodiment, comprises one or more PEST sequences.

In another embodiment, the LLO fragment contains residues of a homologous LLO protein that correspond to one of the above AA ranges. The residue numbers need not, in another embodiment, correspond exactly with the residue numbers enumerated above; e.g. if the homologous LLO protein has an insertion or deletion, relative to an LLO protein utilized herein.

In one embodiment, the present invention contemplates the use of additional nucleic acids that are to be inserted in the Listeria genome along with the episomaly expressed recombinant nucleic acid encoding at least two antigens. In another embodiment, a recombinant Listeria strain of the methods and compositions provided herein further comprises a nucleic acid molecule operably integrated into the Listeria genome as an open reading frame with an endogenous ActA sequence. In another embodiment, an episomal expression vector provided herein comprises a fusion protein comprising the at least two antigens fused to an ActA or a truncated ActA. In one embodiment, the antigen is HMW-MAA, while in another embodiment, it's an immunogenic fragment of HMW-MAA.

In one embodiment, the Listeria genome comprises a deletion of the endogenous ActA gene, which in one embodiment is a virulence factor. In one embodiment, such a deletion provides a more attenuated and thus safer Listeria strain for human use. According to this embodiment, the antigenic polypeptide encoded by the nucleic acid that may be integrated along with the episomal recombinant nucleic acid provided herein is integrated in frame with LLO in the Listeria chromosome. In another embodiment, the integrated nucleic acid molecule is integrated into the ActA locus. In another embodiment, the chromosomal nucleic acid encoding ActA is replaced by a nucleic acid molecule encoding an antigen.

Techniques for homologous recombination are well known in the art, and are described, for example, in Frankel, F R, Hegde, S, Lieberman, J, and Y Paterson. Induction of a cell-mediated immune response to HIV gag using Listeria monocytogenes as a live vaccine vector. J. Immunol. 155: 4766-4774. 1995; Mata, M, Yao, Z, Zubair, A, Syres, K and Y Paterson, Evaluation of a recombinant Listeria monocytogenes expressing an HIV protein that protects mice against viral challenge. Vaccine 19:1435-45, 2001; Boyer, J D, Robinson, T M, Maciag, P C, Peng, X, Johnson, R S, Pavlakis, G, Lewis, M G, Shen, A, Siliciano, R, Brown, C R, Weiner, D, and Y Paterson. DNA prime Listeria boost induces a cellular immune response to SIV antigens in the Rhesus Macaque model that is capable of limited suppression of SIV239 viral replication. Virology. 333: 88-101, 2005. In another embodiment, homologous recombination is performed as described in U.S. Pat. No. 6,855,320. In another embodiment, a temperature sensitive plasmid is used to select the recombinants. Each technique represents a separate embodiment of the methods and compositions provided herein.

In another embodiment, the construct or heterologous gene is integrated into the Listerial chromosome using transposon insertion. Techniques for transposon insertion are well known in the art, and are described, inter alia, by Sun et al. (Infection and Immunity 1990, 58: 3770-3778) in the construction of DP-L967. Transposon mutagenesis has the advantage, in one embodiment, that a stable genomic insertion mutant can be formed. In another embodiment, the position in the genome where the foreign gene has been inserted by transposon mutagenesis is unknown.

In another embodiment, the nucleic acid construct or heterologous gene that is to be integrated into the Listerial chromosome of a Listeria having the episomal recombinant nucleic acid provided herein, is integrated using phage integration sites (Lauer P, Chow M Y et al, Construction, characterization, and use of two LM site-specific phage integration vectors. J Bacteriol 2002; 184(15): 4177-86). In another embodiment, an integrase gene and attachment site of a bacteriophage (e.g. U153 or PSA listeriophage) is used to insert the heterologous gene into the corresponding attachment site, which can be any appropriate site in the genome (e.g. comK or the 3′ end of the arg tRNA gene). In another embodiment, endogenous prophages are cured from the attachment site utilized prior to integration of the construct or heterologous gene. In another embodiment, this method results in single-copy integrants. Each possibility represents a separate embodiment provided herein.

In one embodiment, the nucleic acid construct used for integration to the Listeria genome contains an integration site. In one embodiment, the site is a PhSA (phage from Scott A) attPP′ integration site. PhSA is, in another embodiment, the prophage of L. monocytogenes strain ScottA (Loessner, M. J., I. B. Krause, T. Henle, and S. Scherer. 1994. Structural proteins and DNA characteristics of 14 Listeria typing bacteriophages. J. Gen. Virol. 75:701-710, incorporated herein by reference), a serotype 4b strain that was isolated during an epidemic of human listeriosis. In another embodiment, the site is any another integration site known in the art. Each possibility represents a separate embodiment of the methods and compositions provided herein.

In another embodiment, the nucleic acid construct contains an integrase gene. In another embodiment, the integrase gene is a PhSA integrase gene. In another embodiment, the integrase gene is any other integrase gene known in the art. Each possibility represents a separate embodiment of the methods and compositions provided herein.

In one embodiment, the nucleic acid construct is a plasmid. In another embodiment, the nucleic acid construct is a shuttle plasmid. In another embodiment, the nucleic acid construct is an integration vector. In another embodiment, the nucleic acid construct is a site-specific integration vector. In another embodiment, the nucleic acid construct is any other type of nucleic acid construct known in the art. Each possibility represents a separate embodiment of the methods and compositions provided herein.

The integration vector of methods and compositions provided herein is, in another embodiment, a phage vector. In another embodiment, the integration vector is a site-specific integration vector. In another embodiment, the vector further comprises an attPP′ site. Each possibility represents a separate embodiment of the methods and compositions provided herein.

In another embodiment, the integration vector is a U153 vector. In another embodiment, the integration vector is an A118 vector. In another embodiment, the integration vector is a PhSA vector.

In another embodiment, the vector is an A511 vector (e.g. GenBank Accession No: X91069). In another embodiment, the vector is an A006 vector. In another embodiment, the vector is a B545 vector. In another embodiment, the vector is a B053 vector. In another embodiment, the vector is an A020 vector. In another embodiment, the vector is an A500 vector (e.g. GenBank Accession No: X85009). In another embodiment, the vector is a B051 vector. In another embodiment, the vector is a B052 vector. In another embodiment, the vector is a B054 vector. In another embodiment, the vector is a B055 vector. In another embodiment, the vector is a B056 vector. In another embodiment, the vector is a B101 vector. In another embodiment, the vector is a B110 vector. In another embodiment, the vector is a B111 vector. In another embodiment, the vector is an A153 vector. In another embodiment, the vector is a D441 vector. In another embodiment, the vector is an A538 vector. In another embodiment, the vector is a B653 vector. In another embodiment, the vector is an A513 vector. In another embodiment, the vector is an A507 vector. In another embodiment, the vector is an A502 vector. In another embodiment, the vector is an A505 vector. In another embodiment, the vector is an A519 vector. In another embodiment, the vector is a B604 vector. In another embodiment, the vector is a C703 vector. In another embodiment, the vector is a B025 vector. In another embodiment, the vector is an A528 vector. In another embodiment, the vector is a B024 vector. In another embodiment, the vector is a B012 vector.

In another embodiment, the vector is a B035 vector. In another embodiment, the vector is a C707 vector.

In another embodiment, the integration vector is any other site-specific integration vector known in the art that is capable of infecting Listeria. Each possibility represents a separate embodiment of the methods and compositions provided herein. In another embodiment, the integration vector or the episomal recombinant nucleic acid of the methods and compositions provided herein does not confer antibiotic resistance to the Listeria vaccine strain. In another embodiment, the integration vector or plasmid does not contain an antibiotic resistance gene. Each possibility represents a separate embodiment of the methods and compositions provided herein.

In one embodiment, an antigen of the methods and compositions provided herein is fused to an ActA protein, which in one embodiment, is an N-terminal fragment of an ActA protein, which in one embodiment, comprises or consists of the first 390 AA of ActA, in another embodiment, the first 418 AA of ActA, in another embodiment, the first 50 AA of ActA, in another embodiment, the first 100 AA of ActA, which in one embodiment, comprise a PEST sequence such as that provided in SEQ ID NO: 2. In another embodiment, an N-terminal fragment of an ActA protein utilized in methods and compositions provided herein comprises or consists of the first 150 AA of ActA, in another embodiment, the first approximately 200 AA of ActA, which in one embodiment comprises 2 PEST sequences as described herein. In another embodiment, an N-terminal fragment of an ActA protein utilized in methods and compositions provided herein comprises or consists of the first 250 AA of ActA, in another embodiment, the first 300 AA of ActA. In another embodiment, the ActA fragment contains residues of a homologous ActA protein that correspond to one of the above AA ranges. The residue numbers need not, in another embodiment, correspond exactly with the residue numbers enumerated above; e.g. if the homologous ActA protein has an insertion or deletion, relative to an ActA protein utilized herein, then the residue numbers can be adjusted accordingly, as would be routine to a skilled artisan using sequence alignment tools such as NCBI BLAST that are well-known in the art.

In another embodiment, the N-terminal portion of the ActA protein comprises 1, 2, 3, or 4 PEST sequences, which in one embodiment are the PEST sequences specifically mentioned herein, or their homologs, as described herein or other PEST sequences as can be determined using the methods and algorithms described herein or by using alternative methods known in the art.

An N-terminal fragment of an ActA protein utilized in methods and compositions provided herein has, in another embodiment, the sequence set forth in SEQ ID NO: 13:

MRAMMVVFITANCITINPDIIFAATDSEDSSLNTDEWEEEKTEEQPSEVNTGPRYETA REVSSRDIKELEKSNKVRNTNKADLIAMLKEKAEKGPNINNNNSEQTENAAINEEAS GADRPAIQVERRHPGLPSDSAAEIKKRRKAIASSDSELESLTYPDKPTKVNKKKVAKE SVADASESDLDSSMQSADESSPQPLKANQQPFFPKVFKKIKDAGKWVRDKIDENPEV KKAIVDKSAGLIDQLLTKKKSEEVNASDFPPPPTDEELRLALPETPMLLGFNAPATSEP SSFEFPPPPTDEELRLALPETPMLLGFNAPATSEPSSFEFPPPPTEDELEIIRETASSLD SS FTRGDLASLRNAINRHSQNFSDFPPIPTEEELNGRGGRP (SEQ ID NO: 13). In another embodiment, the ActA fragment comprises the sequence set forth in SEQ ID NO: 13. In another embodiment, the ActA fragment is any other ActA fragment known in the art. In another embodiment, the ActA protein is a homologue of SEQ ID NO: 13. In another embodiment, the ActA protein is a variant of SEQ ID NO: 13. In another embodiment, the ActA protein is an isoform of SEQ ID NO: 13. In another embodiment, the ActA protein is a fragment of SEQ ID NO: 13. In another embodiment, the ActA protein is a fragment of a homologue of SEQ ID NO: 13. In another embodiment, the ActA protein is a fragment of a variant of SEQ ID NO: 13. In another embodiment, the ActA protein is a fragment of an isoform of SEQ ID NO: 13. Each possibility represents a separate embodiment provided herein. Each possibility represents a separate embodiment provided herein.

In another embodiment, the recombinant nucleotide encoding a fragment of an ActA protein comprises the sequence set forth in SEQ ID NO: 14: atgcgtgcgatgatggtggttttcattactgccaattgcattacgattaaccccgacataatatttgcagcgacagatagcgaagattcta gtctaaacacagatgaatgggaagaagaaaaaacagaagagcaaccaagcgaggtaaatacgggaccaagatacgaaactgcac gtgaagtaagttcacgtgatattaaagaactagaaaaatcgaataaagtgagaaatacgaacaaagcagacctaatagcaatgttgaa agaaaaagcagaaaaaggtccaaatatcaataataacaacagtgaacaaactgagaatgcggctataaatgaagaggcttcaggag ccgaccgaccagctatacaagtggagcgtcgtcatccaggattgccatcggatagcgcagcggaaattaaaaaaagaaggaaagcc atagcatcatcggatagtgagcttgaaagccttacttatccggataaaccaacaaaagtaaataagaaaaaagtggcgaaagagtcag ttgcggatgcttctgaaagtgacttagattctagcatgcagtcagcagatgagtcttcaccacaacctttaaaagcaaaccaacaacc at ttttccctaaagtatttaaaaaaataaaagatgcggggaaatgggtacgtgataaaatcgacgaaaatcctgaagtaaagaaagcgatt gttgataaaagtgcagggttaattgaccaattattaaccaaaaagaaaagtgaagaggtaaatgcttcggacttcccgccaccacctac ggatgaagagttaagacttgctttgccagagacaccaatgcttcttggttttaatgctcctgctacatcagaaccgagctcattcgaatttc caccaccacctacggatgaagagttaagacttgctttgccagagacgccaatgcttcttggttttaatgctcctgctacatcggaaccga gctcgttcgaatttccaccgcctccaacagaagatgaactagaaatcatccgggaaacagcatcctcgctagattctagttttacaagag gggatttagctagtttgagaaatgctattaatcgccatagtcaaaatttctctgatttcccaccaatcccaacagaagaagagttgaacgg gagaggcggtagacca (SEQ ID NO: 14). In another embodiment, the recombinant nucleotide has the sequence set forth in SEQ ID NO: 14. In another embodiment, the recombinant nucleotide comprises any other sequence that encodes a fragment of an ActA protein. Each possibility represents a separate embodiment of the methods and compositions provided herein.

An N-terminal fragment of an ActA protein utilized in methods and compositions provided herein has, in another embodiment, the sequence set forth in SEQ ID NO: 15: MRAMMVVFITANCITINPDIIFAATDSEDSSLNTDEWEEEKTEEQPSEVNTGPRYE TAREVSSRDIEELEKSNKVKNTNKADLIAMLKAKAEKGPNNNNNNGEQTGNVAINE EASGVDRPTLQVERRHPGLSSDSAAEIKKRRKAIASSDSELESLTYPDKPTKANKRKV AKESVVDASESDLDSSMQSADESTPQPLKANQKPFFPKVFKKIKDAGKWVRDKIDE NPEVKKAIVDKSAGLIDQLLTKKKSEEVNASDFPPPPTDEELRLALPETPMLLGFNAP TPSEPSSFEFPPPPTDEELRLALPETPMLLGFNAPATSEPSSFEFPPPPTEDELEIMRETA PSLDSSFTSGDLASLRSAINRHSENFSDFPLIPTEEELNGRGGRP (SEQ ID NO: 15), which in one embodiment is the first 390 AA for ActA from Listeria monocytogenes, strain 10403S. In another embodiment, the ActA fragment comprises the sequence set forth in SEQ ID NO: 15. In another embodiment, the ActA fragment is any other ActA fragment known in the art. In another embodiment, the ActA protein is a homologue of SEQ ID NO: 15. In another embodiment, the ActA protein is a variant of SEQ ID NO: 15. In another embodiment, the ActA protein is an isoform of SEQ ID NO: 15. In another embodiment, the ActA protein is a fragment of SEQ ID NO: 15. In another embodiment, the ActA protein is a fragment of a homologue of SEQ ID NO: 15. In another embodiment, the ActA protein is a fragment of a variant of SEQ ID NO: 15. In another embodiment, the ActA protein is a fragment of an isoform of SEQ ID NO: 15. Each possibility represents a separate embodiment of the methods and compositions provided herein.

In another embodiment, the recombinant nucleotide encoding a fragment of an ActA protein comprises the sequence set forth in SEQ ID NO: 16: atgcgtgcgatgatggtagttttcattactgccaactgcattacgattaaccccgacataatatttgcagcgacagatagcgaagattccag tctaaacacagatgaatgggaagaagaaaaaacagaagagcagccaagcgaggtaaatacgggaccaagatacgaaactgcacgt gaagtaagttcacgtgatattgaggaactagaaaaatcgaataaagtgaaaaatacgaacaaagcagacctaatagcaatgttgaaagc aaaagcagagaaaggtccgaataacaataataacaacggtgagcaaacaggaaatgtggctataaatgaagaggcttcaggagtcga ccgaccaactctgcaagtggagcgtcgtcatccaggtctgtcatcggatagcgcagcggaaattaaaaaaagaagaaaagccatagc gtcgtcggatagtgagatgaaagccttacttatccagataaaccaacaaaagcaaataagagaaaagtggcgaaagagtcagttgtgg atgcttctgaaagtgacttagattctagcatgcagtcagcagacgagtctacaccacaacctttaaaagcaaatcaaaaaccatttttccct aaagtatttaaaaaaataaaagatgcggggaaatgggtacgtgataaaatcgacgaaaatcctgaagtaaagaaagcgattgttgataa aagtgcagggttaattgaccaattattaaccaaaaagaaaagtgaagaggtaaatgatcggacttcccgccaccacctacggatgaag agttaagacttgattgccagagacaccgatgatctcggttttaatgctcctactccatcggaaccgagctcattcgaatttccgccgcca cctacggatgaagagttaagacttgattgccagagacgccaatgatcttggttttaatgctcctgctacatcggaaccgagctcattcga atttccaccgcctccaacagaagatgaactagaaattatgcgggaaacagcaccttcgctagattctagttttacaagcggggatttagct agtttgagaagtgctattaatcgccatagcgaaaatttctctgatttcccactaatcccaacagaagaagagttgaacgggagaggcggt agacca (SEQ ID NO: 16), which in one embodiment, is the first 1170 nucleotides encoding ActA in Listeria monocytogenes 10403S strain. In another embodiment, the recombinant nucleotide has the sequence set forth in SEQ ID NO: 16. In another embodiment, the recombinant nucleotide comprises any other sequence that encodes a fragment of an ActA protein. Each possibility represents a separate embodiment of the methods and compositions provided herein.

In another embodiment, the ActA fragment is another ActA fragment known in the art, which in one embodiment, is any fragment comprising a PEST sequence. Thus, in one embodiment, the ActA fragment is amino acids 1-100 of the ActA sequence. In another embodiment, the ActA fragment is amino acids 1-200 of the ActA sequence. In another embodiment, the ActA fragment is amino acids 200-300 of the ActA sequence. In another embodiment, the ActA fragment is amino acids 300-400 of the ActA sequence. In another embodiment, the ActA fragment is amino acids 1-300 of the ActA sequence. In another embodiment, a recombinant nucleotide provided herein comprises any other sequence that encodes a fragment of an ActA protein. In another embodiment, the recombinant nucleotide comprises any other sequence that encodes an entire ActA protein. Each possibility represents a separate embodiment of the methods and compositions provided herein.

In one embodiment, the ActA sequence for use in the compositions and methods provided herein is from Listeria monocytogenes, which in one embodiment, is the EGD strain, the 10403S strain (Genbank accession number: DQ054585) the NICPBP 54002 strain (Genbank accession number: EU394959), the S3 strain (Genbank accession number: EU394960), the NCTC 5348 strain (Genbank accession number: EU394961), the NICPBP 54006 strain (Genbank accession number: EU394962), the M7 strain (Genbank accession number: EU394963), the S19 strain (Genbank accession number: EU394964), or any other strain of Listeria monocytogenes which is known in the art.

In one embodiment, the sequence of the deleted actA region in the strain, LmddΔactA is as follows:

gcgccaaatcattggttgattggtgaggatgtctgtgtgcgtgggtcgcgagatgggcgaataagaagcattaaagatcct gacaaatataatcaagcggctcatatgaaagattacgaatcgcttccactcacagaggaaggcgactggggcggagttcattataatag tggtatcccgaataaagcagcctataatactatcactaaacttggaaaagaaaaaacagaacagctttattdcgcgccttaaagtactattt aacgaaaaaatcccagtttaccgatgcgaaaaaagcgcttcaacaagcagcgaaagatttatatggtgaagatgcttctaaaaaagttgc tgaagcttgggaagcagttggggttaactgattaacaaatgttagagaaaaattaattctccaagtgatattcttaaaataattcatgaatatt ttttcttatattagctaattaagaagataactaactgctaatccaatttttaacggaacaaattagtgaaaatgaaggccgaattttccttgttct aaaaaggttgtattagcgtatcacgaggagggagtataagtgggattaaacagatttatgcgtgcgatgatggtggttttcattactgcca attgcattacgattaaccccgacgIcgacccatacgacgttaattcttgcaatgttagctattggcgtgttctctttaggggcgtttatcaaaa ttattcaattaagaaaaaataattaaaaacacagaacgaaagaaaaagtgaggtgaatgatatgaaattcaaaaaggtggttctaggtatg tgcttgatcgcaagtgttctagtctaccggtaacgataaaagcaaatgcctgttgtgatgaatacttacaaacacccgcagctccgcatga tattgacagcaaattaccacataaacttagttggtccgcggataacccgacaaatactgacgtaaatacgcactattggctttttaaacaag cggaaaaaatactagctaaagatgtaaatcatatgcgagctaatttaatgaatgaacttaaaaaattcgataaacaaatagctcaaggaat atatgatgcggatcataaaaatccatattatgatactagtacatttttatctcatttttataatcctgatagagataatacttatttgccgggttttg ctaatgcgaaaataacaggagcaaagtatttcaatcaatcggtgactgattaccgagaagggaa (SEQ ID NO: 17). In one embodiment, the underlined region contains actA sequence element that is present in the LmddΔactA strain. In one embodiment, the bold sequence gtcgac represent the site of junction of the N-T and C-T sequence.

In one embodiment, the recombinant Listeria strain of the compositions and methods provided herein comprise a first or second nucleic acid molecule that encodes a High Molecular Weight-Melanoma Associated Antigen (HMW-MAA), or, in another embodiment, a fragment of HMW-MAA.

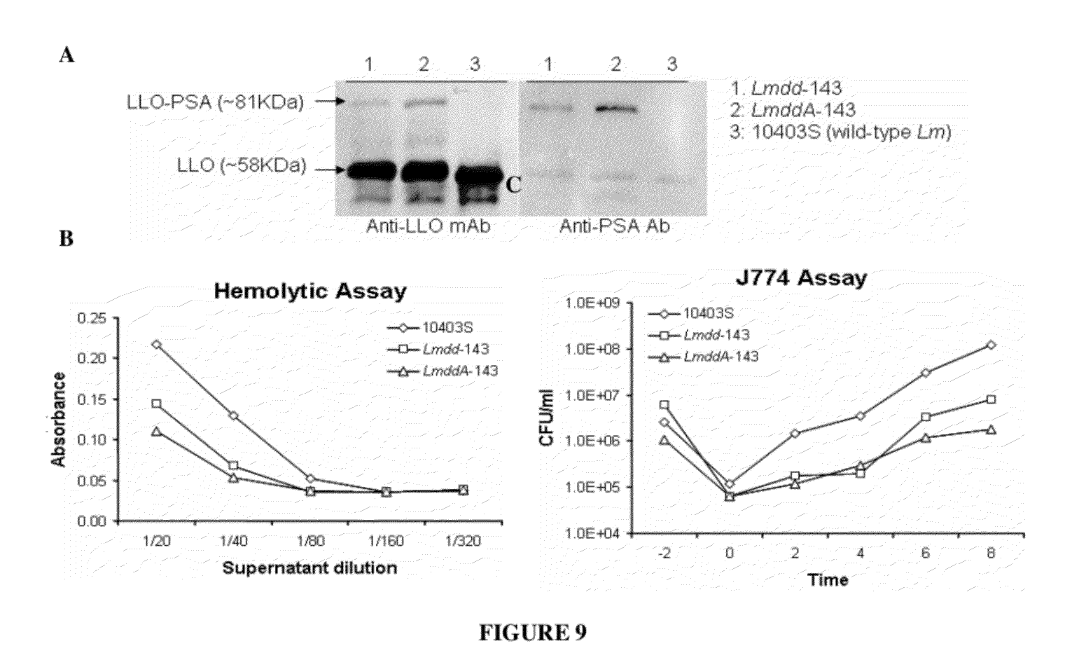

In one embodiment, HMW-MAA is also known as the melanoma chondroitin sulfate proteoglycan (MCSP), and in another embodiment, is a membrane-bound protein of 2322 residues. In one embodiment, HMW-MAA is expressed on over 90% of surgically removed benign nevi and melanoma lesions, and is also expressed in basal cell carcinoma, tumors of neural crest origin (e.g. astrocytomas, gliomas, neuroblastomas and sarcomas), childhood leukemias, and lobular breast carcinoma lesions. In another embodiment, HMW-MAA is highly expressed on both activated pericytes and pericytes in tumor angiogeneic vasculature which, in another embodiment is associated with neovascularization in vivo. In another embodiment, immunization of mice with the recombinant Listeria, provided herein, that expresses a fragment of HMW-MAA (residues 2160 to 2258), impairs the growth of tumors not engineered to express HMW-MAA (FIG. 9D). In another embodiment, immunization of mice with the recombinant Listeria expressing a fragment of HMW-MAA (residues 2160 to 2258) decreases the number of pericytes in the tumor vasculature. In another embodiment, immunization of mice with the recombinant Listeria expressing a fragment of HMW-MAA (residues 2160 to 2258) causes infiltration of CD8⁺ T cells around blood vessels and into the tumor. In another embodiment, HMW-MAA is highly expressed on both activated pericytes and pericytes in tumor angiogenic vasculature. In one embodiment, activated pericytes are associated with neovascularization in vivo. In one embodiment, activated pericytes are involved in angiogenesis. In another embodiment, angiogenesis is important for survival of tumors. In another embodiment, pericytes in tumor angiogenic vasculature are associated with neovascularization in vivo. In another embodiment, activated pericytes are important cells in vascular development, stabilization, maturation and remodeling. Therefore, in one embodiment, besides its role as a tumor-associated antigen, HMW-MAA is also a potential universal target for anti-angiogenesis using an immunotherapeutic approach provided herein. As described herein (Example 8), results obtained using an Lm-based vaccine against this antigen has supported this possibility.

In another embodiment, one of the antigens of the methods and compositions provided herein is expressed in activated pericytes. In another embodiment, at least one of the antigens is expressed in activated pericytes.

The HMW-MAA protein from which HMW-MAA fragments provided herein are derived is, in another embodiment, a human HMW-MAA protein. In another embodiment, the HMW-MAA protein is a mouse protein. In another embodiment, the HMW-MAA protein is a rat protein. In another embodiment, the HMW-MAA protein is a primate protein. In another embodiment, the HMW-MAA protein is from any other species known in the art. In another embodiment, the HMW-MAA protein is melanoma chondroitin sulfate proteoglycan (MCSP). In another embodiment, an AN2 protein is used in methods and compositions provided herein. In another embodiment, an NG2 protein is used in methods and compositions provided herein.

In another embodiment, the HMW-MAA protein of methods and compositions provided herein has the sequence:

MQSGRGPPLPAPGLALALTLTMLARLASAASFFGENHLEVPVATALTDIDLQ LQFSTSQPEALLLLAAGPADHLLLQLYSGRLQVRLVLGQEELRLQTPAETLLSDSIPHT VVLTVVEGWATLSVDGFLNASSAVPGAPLEVPYGLFVGGTGTLGLPYLRGTSRPLRG CLHAATLNGRSLLRPLTPDVHEGCAEEFSASDDVALGFSGPHSLAAFPAWGTQDEGT LEFTLTTQSRQAPLAFQAGGRRGDFIYVDIFEGHLRAVVEKGQGTVLLHNSVPVADG QPHEVSVHINAHRLEISVDQYPTHTSNRGVLSYLEPRGSLLLGGLDAEASRHLQEHRL GLTPEATNASLLGCMEDLSVNGQRRGLREALLTRNMAAGCRLEEEEYEDDAYGHYE AFSTLAPEAWPAMELPEPCVPEPGLPPVFANFTQLLTISPLVVAEGGTAWLEWRHVQP TLDLMEAELRKSQVLFSVTRGARHGELELDIPGAQARKMFTLLDVVNRKARFIHDGS EDTSDQLVLEVSVTARVPMPSCLRRGQTYLLPIQVNPVNDPPHIIFPHGSLMVILEHTQ KPLGPEVFQAYDPDSACEGLTFQVLGTSSGLPVERRDQPGEPATEFSCRELEAGSLVY VHRGGPAQDLTFRVSDGLQASPPATLKVVAIRPAIQIHRSTGLRLAQGSAMPILPANLS VETNAVGQDVSVLFRVTGALQFGELQKQGAGGVEGAEWWATQAFHQRDVEQGRV RYLSTDPQHHAYDTVENLALEVQVGQEILSNLSFPVTIQRATVWMLRLEPLHTQNTQ QETLTTAHLEATLEEAGPSPPTFHYEVVQAPRKGNLQLQGTRLSDGQGFTQDDIQAG RVTYGATARASEAVEDTFRFRVTAPPYFSPLYTFPIHIGGDPDAPVLTNVLLVVPEGG EGVLSADHLFVKSLNSASYLYEVMERPRHGRLAWRGTQDKTTMVTSFTNEDLLRGR LVYQHDDSETTEDDIPFVATRQGESSGDMAWEEVRGVFRVAIQPVNDHAPVQTISRIF HVARGGRRLLTTDDVAFSDADSGFADAQLVLTRKDLLFGSIVAVDEPTRPIYRFTQED LRKRRVLFVHSGADRGWIQLQVSDGQHQATALLEVQASEPYLRVANGSSLVVPQGG QGTIDTAVLHLDTNLDIRSGDEVHYHVTAGPRWGQLVRAGQPATAFSQQDLLDGAV LYSHNGSLSPRDTMAFSVEAGPVHTDATLQVTIALEGPLAPLKLVRHKKIYVFQGEA AEIRRDQLEAAQEAVPPADIVFSVKSPPSAGYLVMVSRGALADEPPSLDPVQSFSQEA VDTGRVLYLHSRPEAWSDAFSLDVASGLGAPLEGVLVELEVLPAAIPLEAQNFSVPEG GSLTLAPPLLRVSGPYFPTLLGLSLQVLEPPQHGALQKEDGPQARTLSAFSWRMVEEQ LIRYVHDGSETLTDSFVLMANASEMDRQSHPVAFTVTVLPVNDQPPILTTNTGLQMW EGATAPIPAEALRSTDGDSGSEDLVYTIEQPSNGRVVLRGAPGTEVRSFTQAQLDGGL VLFSHRGTLDGGFRFRLSDGEHTSPGHFFRVTAQKQVLLSLKGSQTLTVCPGSVQPLS SQTLRASSSAGTDPQLLLYRVVRGPQLGRLFHAQQDSTGEALVNFTQAEVYAGNILY EHEMPPEPFWEAHDTLELQLSSPPARDVAATLAVAVSFEAACPQRPSHLWKNKGLW VPEGQRARITVAALDASNLLASVPSPQRSEHDVLFQVTQFPSRGQLLVSEEPLHAGQP HFLQSQLAAGQLVYAHGGGGTQQDGFHFRAHLQGPAGASVAGPQTSEAFAITVRDV NERPPQPQASVPLRLTRGSRAPISRAQLSVVDPDSAPGEIEYEVQRAPHNGFLSLVGG GLGPVTRFTQADVDSGRLAFVANGSSVAGIFQLSMSDGASPPLPMSLAVDILPSAIEV QLRAPLEVPQALGRSSLSQQQLRVVSDREEPEAAYRLIQGPQYGHLLVGGRPTSAFSQ FQIDQGEVVFAFTNFSSSHDHFRVLALARGVNASAVVNVTVRALLHVWAGGPWPQG ATLRLDPTVLDAGELANRTGSVPRFRLLEGPRHGRVVRVPRARTEPGGSQLVEQFTQ QDLEDGRLGLEVGRPEGRAPGPAGDSLTLELWAQGVPPAVASLDFATEPYNAARPYS VALLSVPEAARTEAGKPESSTPTGEPGPMASSPEPAVAKGGFLSFLEANMFSVIIPMCL VLLLLALILPLLFYLRKRNKTGKHDVQVLTAKPRNGLAGDTETFRKVEPGQAIPLTAV PGQGPPPGGQPDPELLQFCRTPNPALKNGQYWV (SEQ ID No: 18). In another embodiment, an HMW-MAA AA sequence of methods and compositions provided herein comprises the sequence set forth in SEQ ID No: 18. In another embodiment, the HMW-MAA AA sequence is a homologue of SEQ ID No: 18. In another embodiment, the HMW-MAA AA sequence is a variant of SEQ ID No: 18. In another embodiment, the HMW-MAA AA sequence is a fragment of SEQ ID No: 18. In another embodiment, the HMW-MAA AA sequence is an isoform of SEQ ID No: 18. Each possibility represents a separate embodiment of the methods and compositions provided herein.

In another embodiment, the HMW-MAA protein of methods and compositions provided herein is encoded by the sequence:

atgcagtccggccgcggccccccacttccagcccccggcctggccttggctttgaccctgactatgttggccagacttgc atccgcggcttccttcttcggtgagaaccacctggaggtgcctgtggccacggctctgaccgacatagacctgcagctgcagttctcca cgtcccagcccgaagccctccttctcctggcagcaggcccagctgaccacctcctgctgcagctctactctggacgcctgcaggtcag acttgttctgggccaggaggagctgaggctgcagactccagcagagacgctgctgagtgactccatcccccacactgtggtgctgact gtcgtagagggctgggccacgttgtcagtcgatgggtttctgaacgcctcctcagcagtcccaggagcccccctagaggtcccctatg ggctctttgttgggggcactgggacccttggcctgccctacctgaggggaaccagccgacccctgaggggttgcctccatgcagcca ccctcaatggccgcagcctcctccggcctctgacccccgatgtgcatgagggctgtgctgaagagttttctgccagtgatgatgtggcc ctgggcttctctgggccccactctctggctgccttccctgcctggggcactcaggacgaaggaaccctagagtttacactcaccacaca gagccggcaggcacccttggccttccaggcagggggccggcgtggggacttcatctatgtggacatatttgagggccacctgcgggc cgtggtggagaagggccagggtaccgtattgctccacaacagtgtgcctgtggccgatgggcagccccatgaggtcagtgtccacat caatgctcaccggctggaaatctccgtggaccagtaccctacgcatacttcgaaccgaggagtcctcagctacctggagccacgggg cagtctecttctcggggggctggatgcagaggcctctcgtcacctccaggaacaccgcctgggcctgacaccagaggccaccaatgc ctccctgctgggctgcatggaagacctcagtgtcaatggccagaggcgggggctgcgggaagctttgctgacgcgcaacatggcag ccggctgcaggctggaggaggaggagtatgaggacgatgcctatggacattatgaagctttctccaccctggcccctgaggcttggcc agccatggagctgcctgagccatgcgtgcctgagccagggctgcctcctgtctttgccaatttcaccecagctgctgactatcagcccact ggtggtggccgaggggggcacagcctggcttgagtggaggcatgtgcagcccacgctggacctgatggaggctgagctgcgcaaa tcccaggtgctgttcagcgtgacccgaggggcacgccatggcgagctcgagctggacatcccgggagcccaggcacgaaaaatgtt caccctcctggacgtggtgaaccgcaaggcccgcttcatccacgatggctctgaggacacctccgaccagctggtgctggaggtgtc ggtgacggctcgggtgcccatgccctcatgccttcggaggggccaaacatacctcctgcccatccaggtcaaccctgtcaatgaccca ccccacatcatcttcccacatggcagcctcatggtgatcctggaacacacgcagaagccgctggggcctgaggttttccaggcctatga cccggactctgcctgtgagggcctcaccttccaggtccttggcacctcctctggcctccccgtggagcgccgagaccagcctgggga gccggcgaccgagttctcctgccgggagttggaggccggcagcctagtctatgtccaccgcggtggtcctgcacaggacttgacgttc cgggtcagcgatggactgcaggccagccccccggccacgctgaaggtggtggccatccggccggccatacagatccaccgcagca cagggttgcgactggcccaaggctctgccatgcccatcttgcccgccaacctgtcggtggagaccaatgccgtggggcaggatgtga gcgtgctgttccgcgtcactggggccctgcagtttggggagctgcagaagcagggggcaggtggggtggagggtgctgagtggtgg gccacacaggcgttccaccagcgggatgtggagcagggccgcgtgaggtacctgagcactgacccacagcaccacgcttacgaca ccgtggagaacctggccctggaggtgcaggtgggccaggagatcctgagcaatctgtccttcccagtgaccatccagagagccactg tgtggatgctgcggctggagccactgcacactcagaacacccagcaggagaccctcaccacagcccacctggaggccaccctgga ggaggcaggcccaagccccccaaccttccattatgaggtggttcaggctcccaggaaaggcaaccttcaactacagggcacaaggct gtcagatggccagggcttcacccaggatgacatacaggctggccgggtgacctatggggccacagcacgtgcctcagaggcagtcg aggacaccttccgtttccgtgtcacagctccaccatatttctccccacttcccactctataccttccccatccacattggtggtgacccagatgcgcct gtcctcaccaatgtcctcctcgtggtgcctgagggtggtgagggtgtcctctctgctgaccacctctttgtcaagagtctcaacagtgcca gctacctctatgaggtcatggagcggccccgccatgggaggttggcttggcgtgggacacaggacaagaccactatggtgacatcctt caccaatgaagacctgttgcgtggccggctggtctaccagcatgatgactccgagaccacagaagatgatatcccatttgttgctaccc gccagggcgagagcagtggtgacatggcctgggaggaggtacggggtgtcttccgagtggccatccagcccgtgaatgaccacgc ccctgtgcagaccatcagccggatcttccatgtggcccggggtgggcggcggctgctgactacagacgacgtggccttcagcgatgc tgactcgggctttgctgacgcccagctggtgcttacccgcaaggacctcctctttggcagtatcgtggccgtagatgagcccacgcggc ccatctaccgcttcacccaggaggacctcaggaagaggcgagtactgttcgtgcactcaggggctgaccgtggctggatccagctgc aggtgtccgacgggcaacaccaggccactgcgctgctggaggtgcaggcctcggaaccctacctccgtgtggccaacggctccagc cttgtggtccctcaagggggccagggcaccatcgacacggccgtgctccacctggacaccaacctcgacatccgcagtggggatga ggtccactaccacgtcacagctggccctcgctggggacagctagtccgggctggtcagccagccacagccttctcccagcaggacct gctggatggggccgttctctatagccacaatggcagcctcagcccccgcgacaccatggccttctccgtggaagcagggccagtgca cacggatgccaccctacaagtgaccattgccctagagggcccactggccccactgaagctggtccggcacaagaagatctacgtcttc cagggagaggcagctgagatcagaagggaccagctggaggcagcccaggaggcagtgccacctgcagacatcgtattctcagtga agagcccaccgagtgccggctacctggtgatggtgtcgcgtggcgccttggcagatgagccacccagcctggaccctgtgcagagct tctcccaggaggcagtggacacaggcagggtcctgtacctgcactcccgccctgaggcctggagcgatgccttctcgctggatgtgg cctcaggcctgggtgctcccctcgagggcgtccttgtggagctggaggtgctgcccgctgccatcccactagaggcgcaaaacttcag cgtccctgagggtggcagcctcaccctggcccctccactgctccgtgtctccgggccctacttccccactctcctgggcctcagcctgc aggtgctggagccaccccagcatggagccctgcagaaggaggacggacctcaagccaggaccctcagcgccttctcctggagaat ggtggaagagcagctgatccgctacgtgcatgacgggagcgagacactgacagacagttttgtcctgatggctaatgcctccgagatg gatcgccagagccatcctgtggccttcactgtcactgtcctgcctgtcaatgaccaaccccccatcctcactacaaacacaggcctgca gatgtgggagggggccactgcgcccatccctgcggaggctctgaggagcacggacggcgactctgggtctgaggatctggtctaca ccatcgagcagcccagcaacgggcgggtagtgctgcggggggcgccgggcactgaggtgcgcagcttcacgcaggcccagctgg acggcgggctcgtgctgttctcacacagaggaaccctggatggaggcttccgcttccgcctctctgacggcgagcacacttcccccgg acacttcttccgagtgacggcccagaagcaagtgctcctctcgctgaagggcagccagacactgactgtctgcccagggtccgtccag ccactcagcagtcagaccctcagggccagctccagcgcaggcactgacccccagctcctgctctaccgtgtggtgcggggccccca gctaggccggctgttccacgcccagcaggacagcacaggggaggccctggtgaacttcactcaggcagaggtctacgctgggaata ttctgtatgagcatgagatgccccccgagcccttttgggaggcccatgataccctagagctccagctgtcctcgccgcctgcccgggac gtggccgccacccttgctgtggctgtgtcttttgaggctgcctgtccccagcgccccagccacctctggaagaacaaaggtctctgggt ccccgagggccagcgggccaggatcaccgtggctgctctggatgcctccaatctcttggccagcgttccatcaccccagcgctcaga gcatgatgtgctcttccaggtcacacagttccccagccggggccagctgttggtgtccgaggagcccctccatgctgggcagccccac ttcctgcagtcccagctggctgcagggcagctagtgtatgcccacggcggtgggggcacccagcaggatggcttccactttcgtgccc acctccaggggccagcaggggcctccgtggctggaccccaaacctcagaggcctttgccatcacggtgagggatgtaaatgagcgg ccccctcagccacaggcctctgtcccactccggctcacccgaggctctcgtgcccccatctcccgggcccagctgagtgtggtggacc cagactcagctcctggggagattgagtacgaggtccagcgggcaccccacaacggcttcctcagcctggtgggtggtggcctgggg cccgtgacccgcttcacgcaagccgatgtggattcagggcggctggccttcgtggccaacgggagcagcgtggcaggcatcttccag ctgagcatgtctgatggggccagcccacccctgcccatgtccctggctgtggacatcctaccatccgccatcgaggtgcagctgcggg cacccctggaggtgccccaagctttggggcgctcctcactgagccagcagcagctccgggtggtttcagatcgggaggagccagag gcagcataccgcctcatccagggaccccagtatgggcatctcctggtgggcgggcggcccacctcggccttcagccaattccagata gaccagggcgaggtggtctttgccttcaccaacttctcctcctctcatgaccacttcagagtcctggcactggctaggggtgtcaatgcat cagccgtagtgaacgtcactgtgagggctctgctgcatgtgtgggcaggtgggccatggccccagggtgccaccctgcgcctggacc ccaccgtcctagatgctggcgagctggccaaccgcacaggcagtgtgccgcgcttccgcctcctggagggaccccggcatggccgc gtggtccgcgtgccccgagccaggacggagcccgggggcagccagctggtggagcagttcactcagcaggaccttgaggacggg aggctggggctggaggtgggcaggccagaggggagggcccccggccccgcaggtgacagtctcactctggagctgtgggcacag ggcgtcccgcctgctgtggcctccctggactttgccactgagccttacaatgctgcccggccctacagcgtggccctgctcagtgtccc cgaggccgcccggacggaagcagggaagccagagagcagcacccccacaggcgagccaggccccatggcatccagccctgag cccgctgtggccaagggaggcttcctgagcttccttgaggccaacatgttcagcgtcatcatccccatgtgcctggtacttctgctcctgg cgctcatcctgcccctgctcttctacctccgaaaacgcaacaagacgggcaagcatgacgtccaggtcctgactgccaagccccgcaa cggcctggctggtgacaccgagacctttcgcaaggtggagccaggccaggccatcccgctacagctgtgcctggccaggggcccc ctccaggaggccagcctgacccagagctgcgcagttctgccggacacccaaccctgcccttaagaatggccagtactgggtgtgag gcctggcctgggcccagatgctgatcgggccagggacaggc (SEQ ID No: 19). In another embodiment, the recombinant nucleotide has the sequence set forth in SEQ ID NO: 19 In another embodiment, an HMW-MAA-encoding nucleotide of methods and compositions provided herein comprises the sequence set forth in SEQ ID No: 19. In another embodiment, the HMW-MAA-encoding nucleotide is a homologue of SEQ ID No: 19. In another embodiment, the HMW-MAA-encoding nucleotide is a variant of SEQ ID No: 19. In another embodiment, the HMW-MAA-encoding nucleotide is a fragment of SEQ ID No: 19. In another embodiment, the HMW-MAA-encoding nucleotide is an isoform of SEQ ID No: 19. Each possibility represents a separate embodiment of the methods and compositions provided herein.

In another embodiment, the HMW-MAA protein of methods and compositions provided herein has an AA sequence set forth in a GenBank entry having an Accession Numbers selected from NM_{—}001897 and X96753. In another embodiment, the HMW-MAA protein is encoded by a nucleotide sequence set forth in one of the above GenBank entries. In another embodiment, the HMW-MAA protein comprises a sequence set forth in one of the above GenBank entries. In another embodiment, the HMW-MAA protein is a homologue of a sequence set forth in one of the above GenBank entries. In another embodiment, the HMW-MAA protein is a variant of a sequence set forth in one of the above GenBank entries. In another embodiment, the HMW-MAA protein is a fragment of a sequence set forth in one of the above GenBank entries. In another embodiment, the HMW-MAA protein is an isoform of a sequence set forth in one of the above GenBank entries. Each possibility represents a separate embodiment of the methods and compositions provided herein.

The HMW-MAA fragment utilized in the present invention comprises, in another embodiment, AA 360-554. In another embodiment, the fragment consists essentially of AA 360-554. In another embodiment, the fragment consists of AA 360-554. In another embodiment, the fragment comprises AA701-1130. In another embodiment, the fragment consists essentially of AA 701-1130 In another embodiment, the fragment consists of AA 701-1130 In another embodiment, the fragment comprises AA 2160-2258 In another embodiment, the fragment consists essentially of 2160-2258. In another embodiment, the fragment consists of 2160-2258. Each possibility represents a separate embodiment of the methods and compositions provided herein.

In another embodiment, the recombinant Listeria of the compositions and methods provided herein comprise a plasmid that encodes at least two recombinant polypeptides that are, in one embodiment, angiogenic, and in another embodiment, antigenic. In one embodiment an antigen provided herein is incorporated into an LLO fragment, ActA protein or fragment, or PEST sequence. Each possibility represents a separate embodiment of the methods and compositions provided herein.

In one embodiment, the recombinant Listeria strain of the compositions and methods provided herein expresses a heterologous antigenic polypeptide that is expressed by a tumor cell. In one embodiment, the recombinant Listeria strain of the compositions and methods provided herein comprise a first or second nucleic acid molecule that encodes a Prostate Specific Antigen (PSA), which in one embodiment, is a marker for prostate cancer that is highly expressed by prostate tumors, which in one embodiment is the most frequent type of cancer in American men and, in another embodiment, is the second cause of cancer related death in American men. In one embodiment, PSA is a kallikrein serine protease (KLK3) secreted by prostatic epithelial cells, which in one embodiment, is widely used as a marker for prostate cancer.

In one embodiment, the recombinant Listeria strain provided herein comprises a nucleic acid molecule encoding KLK3 protein.

In another embodiment, the KLK3 protein has the sequence set forth in GenBank Accession No. CAA32915. In another embodiment, the KLK3 protein is a homologue of In another embodiment, the KLK3 protein is a variant of GenBank Accession No. CAA32915.

In another embodiment, the KLK3 protein is an isomer of GenBank Accession No. CAA32915. In another embodiment, the KLK3 protein is a fragment of GenBank Accession No. CAA32915. Each possibility represents a separate embodiment of the methods and compositions provided herein.

In another embodiment, the KLK3 protein has the sequence set forth in GenBank Accession No. AAA59995.1. In another embodiment, the KLK3 protein is a homologue of GenBank Accession No. AAA59995.1. In another embodiment, the KLK3 protein is a variant of GenBank Accession No. AAA59995.1. In another embodiment, the KLK3 protein is an isomer of GenBank Accession No. AAA59995.1. In another embodiment, the KLK3 protein is a fragment of GenBank Accession No. AAA59995.1. Each possibility represents a separate embodiment of the methods and compositions provided herein.

In another embodiment, the KLK3 protein is encoded by a nucleotide molecule having the sequence set forth in GenBank Accession No. X14810). In another embodiment, the KLK3 protein is encoded by residues 401 . . . 446, 1728 . . . 1847, 3477 . . . 3763, 3907 . . . 4043, and 5413 . . . 5572 of GenBank Accession No. X14810. In another embodiment, the KLK3 protein is encoded by a homologue of GenBank Accession No. X14810SEQ. In another embodiment, the KLK3 protein is encoded by a variant of GenBank Accession No. X14810. In another embodiment, the KLK3 protein is encoded by an isomer of GenBank Accession No. X14810.

In another embodiment, the KLK3 protein is encoded by a fragment of GenBank Accession No. X14810. Each possibility represents a separate embodiment of the methods and compositions provided herein.

In another embodiment, the KLK3 protein is encoded by a sequence set forth in one of the following GenBank Accession Numbers: BC005307, AJ310938, AJ310937, AF335478, AF335477, M27274, and M26663. In another embodiment, the KLK3 protein is encoded by a sequence set forth in one of the above GenBank Accession Numbers. Each possibility represents a separate embodiment of the methods and compositions provided herein.

In another embodiment, the KLK3 protein is encoded by a sequence set forth in one of the following GenBank Accession Numbers: NM_{—}001030050, NM_{—}001030049, NM_{—}001030048, NM_{—}001030047, NM_{—}001648, AJ459782, AJ512346, or AJ459784. Each possibility represents a separate embodiment of the methods and compositions provided herein. In one embodiment, the KLK3 protein is encoded by a variation of any of the sequences described herein wherein the sequence lacks MWVPVVFLTLSVTWIGAAPLILSR (SEQ ID NO: 20).

In another embodiment, the KLK3 protein has the sequence that comprises a sequence set forth in one of the following GenBank Accession Numbers: X13943, X13942, X13940, X13941, and X13944. Each possibility represents a separate embodiment of the methods and compositions provided herein.

In another embodiment, the KLK3 protein is any other KLK3 protein known in the art. Each KLK3 protein represents a separate embodiment of the methods and compositions provided herein.

In another embodiment, the KLK3 peptide is any other KLK3 peptide known in the art. In another embodiment, the KLK3 peptide is a fragment of any other KLK3 peptide known in the art. Each type of KLK3 peptide represents a separate embodiment of the methods and compositions provided herein.

“KLK3 peptide” refers, in another embodiment, to a full-length KLK3 protein. In another embodiment, the term refers to a fragment of a KLK3 protein. In another embodiment, the term refers to a fragment of a KLK3 protein that is lacking the KLK3 signal peptide. In another embodiment, the term refers to a KLK3 protein that contains the entire KLK3 sequence except the KLK3 signal peptide. “KLK3 signal sequence” refers, in another embodiment, to any signal sequence found in nature on a KLK3 protein. In another embodiment, a KLK3 protein of methods and compositions provided herein does not contain any signal sequence. Each possibility represents a separate embodiment of the methods and composition provided herein.

In another embodiment, the kallikrein-related peptidase 3 (KLK3 protein) that is the source of a KLK3 peptide for use in the methods and compositions provided herein is a PSA protein. In another embodiment, the KLK3 protein is a P-30 antigen protein. In another embodiment, the KLK3 protein is a gamma-seminoprotein. In another embodiment, the KLK3 protein is a kallikrein 3 protein. In another embodiment, the KLK3 protein is a semenogelase protein. In another embodiment, the KLK3 protein is a seminin protein. In another embodiment, the KLK3 protein is any other type of KLK3 protein that is known in the art. Each possibility represents a separate embodiment of the methods and compositions provided herein.

In another embodiment, the antigen of interest is a KLK9 polypeptide.

In another embodiment, the antigen of interest is HPV-E7. In another embodiment, the antigen is HPV-E6. In another embodiment, the antigen is Her-2/neu. In another embodiment, the antigen is NY-ESO-1. In another embodiment, the antigen is telomerase (TERT). In another embodiment, the antigen is SCCE. In another embodiment, the antigen is CEA. In another embodiment, the antigen is LMP-1. In another embodiment, the antigen is p53. In another embodiment, the antigen is carboxic anhydrase IX (CAIX). In another embodiment, the antigen is PSMA. In another embodiment, the antigen is prostate stem cell antigen (PSCA). In another embodiment, the antigen is HMW-MAA. In another embodiment, the antigen is WT-1. In another embodiment, the antigen is HIV-1 Gag. In another embodiment, the antigen is Proteinase 3. In another embodiment, the antigen is Tyrosinase related protein 2. In another embodiment, the antigen is PSA (prostate-specific antigen). In another embodiment, the antigen is selected from HPV-E7, HPV-E6, Her-2, NY-ESO-1, telomerase (TERT), SCCE, HMW-MAA, WT-1, HIV-1 Gag, CEA, LMP-1, p53, PSMA, PSCA, Proteinase 3, Tyrosinase related protein 2, Muc 1, PSA (prostate-specific antigen), or a combination thereof.

In another embodiment, an antigen provided herein is a tumor-associated antigen, which in one embodiment, is one of the following tumor antigens: a MAGE (Melanoma-Associated Antigen E) protein, e.g. MAGE 1, MAGE 2, MAGE 3, MAGE 4, a tyrosinase; a mutant ras protein; a mutant p53 protein; p97 melanoma antigen, a ras peptide or p53 peptide associated with advanced cancers; the HPV 16/18 antigens associated with cervical cancers, KLH antigen associated with breast carcinoma, CEA (carcinoembryonic antigen) associated with colorectal cancer, gp100, a MART1 antigen associated with melanoma, or the PSA antigen associated with prostate cancer. In another embodiment, the antigen for the compositions and methods provided herein are melanoma-associated antigens, which in one embodiment are TRP-2, MAGE-1, MAGE-3, gp-100, tyrosinase, HSP-70, beta-HCG, or a combination thereof.

In one embodiment, the first and at least second nucleic acids may encode separate antigens that serve as tumor targets, which in one embodiment are Prostate Specific Antigen (PSA) and Prostate Cancer Stem Cell (PSCA) antigen. In one embodiment, the polypeptide encoded by the at least second nucleic acid may complement or synergize the immune response to the first nucleic acid encoding an antigenic polypeptide. In another embodiment, the polypeptide encoded by the at least second nucleic acid affects vascular growth. In one embodiment, the first and at least second nucleic acid may encode two polypeptides that affect vascular growth, which in one embodiment, work via distinct mechanisms to affect vascular growth. In one embodiment, such polypeptides are EGFR-III, HMW-MAA, or a combination thereof. In one embodiment, a polypeptide may serve as both a tumor antigen an angiogenic factor. In one embodiment, the first nucleic acid may encode a tumor antigen, and the at least second nucleic acid may encode a polypeptide that is an inhibitor of the function or expression of ARG-1 or NOS or combination. In one embodiment, an inhibitor of NOS is N^{G}-mono-methyl-L-arginine (L-NMMA), N^{G}-nitro-L-argininemethyl ester (L-NAME), 7-NI, L-NIL, or L-NIO. In one embodiment, N-omega-nitro-L-arginine a nitric oxide synthase inhibitor and L-arginine competitive inhibitor may be encoded by the nucleic acid. In one embodiment, the second nucleic acid may encode an mRNA that inhibits function or expression of ARG-1 or NOS.

In one embodiment, at least one of the polypeptides expressed by the Listeria of the present invention may be a neuropeptide growth factor antagonist, which in one embodiment is [D-Arg1, D-Phe5, D-Trp-7,9, Leu11]substance P, [Arg6, D-Trp-7,9, NmePhe8]substance P(6-11). These and related embodiments are understood by one of skill in the art.

In another embodiment, the antigen is an infectious disease antigen. In one embodiment, the antigen is an auto antigen or a self-antigen.

In other embodiments, the antigen is derived from a fungal pathogen, bacteria, parasite, helminth, or viruses. In other embodiments, the antigen is selected from tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, human papilloma virus antigens E1 and E2 from type HPV-16, -18, -31, -33, -35 or -45 human papilloma viruses, or a combination thereof.

In other embodiments, the antigen is associated with one of the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough3 yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and lesteriosis. Each antigen represents a separate embodiment of the methods and composition provided herein.

The immune response induced by methods and compositions provided herein is, in another embodiment, a T cell response. In another embodiment, the immune response comprises a T cell response. In another embodiment, the response is a CD8+ T cell response. In another embodiment, the response comprises a CD8⁺ T cell response. Each possibility represents a separate embodiment provided herein.

In one embodiment, a recombinant Listeria of the compositions and methods provided herein comprise an angiogenic antigen. In another embodiment, anti-angiogenic therapy targets pericytes. In another embodiment, molecular targets on vascular endothelial cells and pericytes are important targets for antitumor therapies. In another embodiment, the platelet-derived growth factor receptor (PDGF-B/PDGFR-β) signaling is important to recruit pericytes to newly formed blood vessels. Thus, in one embodiment, angiogenic antigens provided herein inhibit molecules involved in pericyte signaling, which in one embodiment, is PDGFR-β.

In one embodiment, the compositions of the present invention comprise an angiogenic factor, or an immunogenic fragment thereof, where in one embodiment, the immunogenic fragment comprises one or more epitopes recognized by the host immune system. In one embodiment, an angiogenic factor is a molecule involved in the formation of new blood vessels. In one embodiment, the angiogenic factor is VEGFR2. In another embodiment, an angiogenic factor of the present invention is Angiogenin; Angiopoietin-1; Del-1; Fibroblast growth factors: acidic (aFGF) and basic (bFGF); Follistatin; Granulocyte colony-stimulating factor (G-CSF); Hepatocyte growth factor (HGF)/scatter factor (SF); Interleukin-8 (IL-8); Leptin; Midkine; Placental growth factor; Platelet-derived endothelial cell growth factor (PD-ECGF); Platelet-derived growth factor-BB (PDGF-BB); Pleiotrophin (PTN); Progranulin; Proliferin; Transforming growth factor-alpha (TGF-alpha); Transforming growth factor-beta (TGF-beta); Tumor necrosis factor-alpha (TNF-alpha); Vascular endothelial growth factor (VEGF)/vascular permeability factor (VPF). In another embodiment, an angiogenic factor is an angiogenic protein. In one embodiment, a growth factor is an angiogenic protein. In one embodiment, an angiogenic protein for use in the compositions and methods of the present invention is Fibroblast growth factors (FGF); VEGF; VEGFR and Neuropilin 1 (NRP-1); Angiopoietin 1 (Ang1) and Tie2; Platelet-derived growth factor (PDGF; BB-homodimer) and PDGFR; Transforming growth factor-beta (TGF-β), endoglin and TGF-β receptors; monocyte chemotactic protein-1 (MCP-1); Integrins αVβ3, αVβ5 and α5β1; VE-cadherin and CD31; ephrin; plasminogen activators; plasminogen activator inhibitor-1; Nitric oxide synthase (NOS) and COX-2; AC133; or Id1/Id3. In one embodiment, an angiogenic protein for use in the compositions and methods of the present invention is an angiopoietin, which in one embodiment, is Angiopoietin 1, Angiopoietin 3, Angiopoietin 4 or Angiopoietin 6. In one embodiment, endoglin is also known as CD105; EDG; HHT1; ORW; or ORW1. In one embodiment, endoglin is a TGF beta co-receptor.

In one embodiment, the compositions and methods provided herein provide anti-angiogenesis therapy, which in one embodiment, may improve immunotherapy strategies. In one embodiment, the compositions and methods provided herein circumvent endothelial cell anergy in vivo by up-regulating adhesion molecules in tumor vessels and enhancing leukocyte-vessel interactions, which increases the number of tumor infiltrating leukocytes, such as CD8⁺ T cells. Interestingly, enhanced anti-tumor protection correlates with an increased number of activated CD4⁺ and CD8⁺ tumor-infiltrating T cells and a pronounced decrease in the number of regulatory T cells in the tumor upon VEGF blockade.

In one embodiment, delivery of anti-angiogenic antigen simultaneously with a tumor-associated antigen to a host afflicted by a tumor has a synergistic effect in impacting tumor growth and a more potent therapeutic efficacy.

In another embodiment, targeting pericytes through vaccination leads to cytotoxic T lymphocyte (CTL) infiltration, destruction of pericytes, blood vessel destabilization and vascular inflammation, which in another embodiment is associated with up-regulation of adhesion molecules in the endothelial cells that are important for lymphocyte adherence and transmigration, ultimately improving the ability of lymphocytes to infiltrate the tumor tissue. In another embodiment, concomitant delivery of a tumor-specific antigen generates lymphocytes able to invade the tumor site and kill tumor cells.

In one embodiment, the platelet-derived growth factor receptor (PDGF-B/PDGFR-β) signaling is important to recruit pericytes to newly formed blood vessels. In another embodiment, inhibition of VEGFR-2 and PDGFR-β concomitantly induces endothelial cell apoptosis and regression of tumor blood vessels, in one embodiment, approximately 40% of tumor blood vessels.

In another embodiment, the recombinant Listeria strain is an auxotrophic Listeria strain. In another embodiment, the auxotrophic Listeria strain is a dal/dat mutant. In another embodiment, the nucleic acid molecule is stably maintained in the recombinant bacterial strain in the absence of antibiotic selection.

In one embodiment, auxotrophic mutants useful as vaccine vectors are generated in a number of ways. In another embodiment, D-alanine auxotrophic mutants are generated, in one embodiment, via the disruption of both the dal gene and the dat gene to generate an attenuated auxotrophic strain of Listeria which requires exogenously added D-alanine for growth.

In one embodiment, the generation of AA strains of Listeria deficient in D-alanine, for example, may be accomplished in a number of ways that are well known to those of skill in the art, including deletion mutagenesis, insertion mutagenesis, and mutagenesis which results in the generation of frame shift mutations, mutations which cause premature termination of a protein, or mutation of regulatory sequences which affect gene expression. In another embodiment, mutagenesis can be accomplished using recombinant DNA techniques or using traditional mutagenesis technology using mutagenic chemicals or radiation and subsequent selection of mutants. In another embodiment, deletion mutants are preferred because of the accompanying low probability of reversion of the auxotrophic phenotype. In another embodiment, mutants of D-alanine which are generated according to the protocols presented herein may be tested for the ability to grow in the absence of D-alanine in a simple laboratory culture assay. In another embodiment, those mutants which are unable to grow in the absence of this compound are selected for further study.

In another embodiment, in addition to the aforementioned D-alanine associated genes, other genes involved in synthesis of a metabolic enzyme, provided herein, may be used as targets for mutagenesis of Listeria.

In one embodiment, the auxotrophic Listeria strain comprises an episomal expression vector comprising a metabolic enzyme that complements the auxotrophy of the auxotrophic Listeria strain. In another embodiment, the construct is contained in the Listeria strain in an episomal fashion. In another embodiment, the foreign antigen is expressed from a vector harbored by the recombinant Listeria strain. In another embodiment, the episomal expression vector lacks an antibiotic resistance marker. In one embodiment, an antigen of the methods and compositions provided herein is genetically fused to an oligopeptide comprising a PEST sequence. In another embodiment, the endogenous polypeptide comprising a PEST sequence is LLO. In another embodiment, the endogenous polypeptide comprising a PEST sequence is ActA. Each possibility represents a separate embodiment of the methods and compositions provided herein.

In another embodiment, the metabolic enzyme complements an endogenous metabolic gene that is lacking in the remainder of the chromosome of the recombinant bacterial strain. In one embodiment, the endogenous metabolic gene is mutated in the chromosome. In another embodiment, the endogenous metabolic gene is deleted from the chromosome. In another embodiment, the metabolic enzyme is an amino acid metabolism enzyme. In another embodiment, the metabolic enzyme catalyzes a formation of an amino acid used for a cell wall synthesis in the recombinant Listeria strain. In another embodiment, the metabolic enzyme is an alanine racemes enzyme. In another embodiment, the metabolic enzyme is a D-amino acid transferase enzyme. Each possibility represents a separate embodiment of the methods and compositions provided herein.

In another embodiment, the metabolic enzyme catalyzes the formation of an amino acid (AA) used in cell wall synthesis. In another embodiment, the metabolic enzyme catalyzes synthesis of an AA used in cell wall synthesis. In another embodiment, the metabolic enzyme is involved in synthesis of an AA used in cell wall synthesis. In another embodiment, the AA is used in cell wall biogenesis. Each possibility represents a separate embodiment of the methods and compositions provided herein.

In another embodiment, the metabolic enzyme is a synthetic enzyme for D-glutamic acid, a cell wall component.

In another embodiment, the metabolic enzyme is encoded by an alanine racemase gene (dal) gene. In another embodiment, the dal gene encodes alanine racemase, which catalyzes the reaction L-alanine [symbol] D-alanine.

The dal gene of methods and compositions of the methods and composition provided herein is encoded, in another embodiment, by the sequence set forth in GenBank Accession No: AF038438). In another embodiment, the nucleotide encoding dal is homologous to GenBank Accession No: AF038438. In another embodiment, the nucleotide encoding dal is a variant of GenBank Accession No: AF038438. In another embodiment, the nucleotide encoding dal is a fragment of GenBank Accession No: AF038438. In another embodiment, the dal protein is encoded by any other dal gene known in the art. Each possibility represents a separate embodiment of the methods and compositions provided herein.

In another embodiment, the dal protein has the sequence set forth in GenBank Accession No: AF038428. In another embodiment, the dal protein is homologous to GenBank Accession No: AF038428. In another embodiment, the dal protein is a variant of GenBank Accession No: AF038428. In another embodiment, the dal protein is an isomer of GenBank Accession No: AF038428. In another embodiment, the dal protein is a fragment of GenBank Accession No: AF038428. In another embodiment, the dal protein is a fragment of a homologue of GenBank Accession No: AF038428. In another embodiment, the dal protein is a fragment of a variant of GenBank Accession No: AF038428. In another embodiment, the dal protein is a fragment or an isomer of GenBank Accession No: AF038428.

In another embodiment, the dal protein is any other Listeria dal protein known in the art. In another embodiment, the dal protein is any other gram-positive dal protein known in the art. In another embodiment, the dal protein is any other dal protein known in the art. Each possibility represents a separate embodiment of the methods and compositions provided herein.

In another embodiment, the dal protein of the methods and compositions provided herein retains its enzymatic activity. In another embodiment, the dal protein retains 90% of wild-type activity. In another embodiment, the dal protein retains 80% of wild-type activity.

In another embodiment, the dal protein retains 70% of wild-type activity. In another embodiment, the dal protein retains 60% of wild-type activity. In another embodiment, the dal protein retains 50% of wild-type activity. In another embodiment, the dal protein retains 40% of wild-type activity. In another embodiment, the dal protein retains 30% of wild-type activity. In another embodiment, the dal protein retains 20% of wild-type activity. In another embodiment, the dal protein retains 10% of wild-type activity. In another embodiment, the dal protein retains 5% of wild-type activity. Each possibility represents a separate embodiment of the methods and compositions provided herein.

In another embodiment, the metabolic enzyme is encoded by a D-amino acid aminotransferase gene (dat). D-glutamic acid synthesis is controlled in part by the dat gene, which is involved in the conversion of D-glu+pyr to alpha-ketoglutarate+D-ala, and the reverse reaction.

In another embodiment, a dat gene utilized in the present invention has the sequence set forth in GenBank Accession Number AF038439. In another embodiment, the dat gene is any another dat gene known in the art. Each possibility represents a separate embodiment of the methods and compositions provided herein.

The dat gene of methods and compositions of the methods and composition provided herein is encoded, in another embodiment, by the sequence set forth in GenBank Accession No: AF038439. In another embodiment, the nucleotide encoding dat is homologous to GenBank Accession No: AF038439. In another embodiment, the nucleotide encoding dat is a variant of GenBank Accession No: AF038439. In another embodiment, the nucleotide encoding dat is a fragment of GenBank Accession No: AF038439. In another embodiment, the dat protein is encoded by any other dat gene known in the art. Each possibility represents a separate embodiment of the methods and compositions provided herein.

In another embodiment, the dat protein has the sequence set forth in GenBank Accession No: AF038439. In another embodiment, the dat protein is homologous to GenBank Accession No: AF038439. In another embodiment, the dat protein is a variant of GenBank Accession No: AF038439. In another embodiment, the dat protein is an isomer of GenBank Accession No: AF038439. In another embodiment, the dat protein is a fragment of GenBank Accession No: AF038439. In another embodiment, the dat protein is a fragment of a homologue of GenBank Accession No: AF038439. In another embodiment, the dat protein is a fragment of an isomer of GenBank Accession No: AF038439.

In another embodiment, the dat protein is any other Listeria dat protein known in the art. In another embodiment, the dat protein is any other gram-positive dat protein known in the art. In another embodiment, the dat protein is any other dat protein known in the art. Each possibility represents a separate embodiment of the methods and compositions provided herein.

In another embodiment, the dat protein of methods and compositions of the methods and compositions provided herein retains its enzymatic activity. In another embodiment, the dat protein retains 90% of wild-type activity. In another embodiment, the dat protein retains 80% of wild-type activity. In another embodiment, the dat protein retains 70% of wild-type activity. In another embodiment, the dat protein retains 60% of wild-type activity. In another embodiment, the dat protein retains 50% of wild-type activity. In another embodiment, the dat protein retains 40% of wild-type activity. In another embodiment, the dat protein retains 30% of wild-type activity. In another embodiment, the dat protein retains 20% of wild-type activity. In another embodiment, the dat protein retains 10% of wild-type activity. In another embodiment, the dat protein retains 5% of wild-type activity. Each possibility represents a separate embodiment of the methods and compositions provided herein.

In another embodiment, the metabolic enzyme is encoded by dga. D-glutamic acid synthesis is also controlled in part by the dga gene, and an auxotrophic mutant for D-glutamic acid synthesis will not grow in the absence of D-glutamic acid (Pucci et al, 1995, J. Bacteriol. 177: 336-342). In another embodiment, the recombinant Listeria is auxotrophic for D-glutamic acid. A further example includes a gene involved in the synthesis of diaminopimelic acid. Such synthesis genes encode beta-semialdehyde dehydrogenase, and when inactivated, renders a mutant auxotrophic for this synthesis pathway (Sizemore et al, 1995, Science 270: 299-302). In another embodiment, the dga protein is any other Listeria dga protein known in the art. In another embodiment, the dga protein is any other gram-positive dga protein known in the art. Each possibility represents a separate embodiment of the methods and compositions provided herein.

In another embodiment, the metabolic enzyme is encoded by an alr (alanine racemase) gene. In another embodiment, the metabolic enzyme is any other enzyme known in the art that is involved in alanine synthesis. In another embodiment, the metabolic enzyme is any other enzyme known in the art that is involved in L-alanine synthesis. In another embodiment, the metabolic enzyme is any other enzyme known in the art that is involved in D-alanine synthesis. In another embodiment, the recombinant Listeria is auxotrophic for D-alanine. Bacteria auxotrophic for alanine synthesis are well known in the art, and are described in, for example, E. coli (Strych et al, 2002, J. Bacteriol. 184:4321-4325), Corynebacterium glutamicum (Tauch et al, 2002, J. Biotechnol 99:79-91), and Listeria monocytogenes (Frankel et al, U.S. Pat. No. 6,099,848)), Lactococcus species, and Lactobacillus species, (Bron et al, 2002, Appl Environ Microbiol, 72: 5663-70). In another embodiment, any D-alanine synthesis gene known in the art is inactivated. Each possibility represents a separate embodiment of the methods and compositions provided herein.

In another embodiment, the metabolic enzyme is an amino acid aminotransferase.

In another embodiment, the metabolic enzyme is encoded by serC, a phosphoserine aminotransferase. In another embodiment, the metabolic enzyme is encoded by asd (aspartate beta-semialdehyde dehydrogenase), involved in synthesis of the cell wall constituent diaminopimelic acid. In another embodiment, the metabolic enzyme is encoded by gsaB-glutamate-1-semialdehyde aminotransferase, which catalyzes the formation of 5-aminolevulinate from (S)-4-amino-5-oxopentanoate. In another embodiment, the metabolic enzyme is encoded by HemL, which catalyzes the formation of 5-aminolevulinate from (S)-4-amino-5-oxopentanoate. In another embodiment, the metabolic enzyme is encoded by aspB, an aspartate aminotransferase that catalyzes the formation of oxalozcetate and L-glutamate from L-aspartate and 2-oxoglutarate. In another embodiment, the metabolic enzyme is encoded by argF-1, involved in arginine biosynthesis. In another embodiment, the metabolic enzyme is encoded by aroE, involved in amino acid biosynthesis. In another embodiment, the metabolic enzyme is encoded by aroB, involved in 3-dehydroquinate biosynthesis. In another embodiment, the metabolic enzyme is encoded by aroD, involved in amino acid biosynthesis. In another embodiment, the metabolic enzyme is encoded by aroC, involved in amino acid biosynthesis. In another embodiment, the metabolic enzyme is encoded by hisB, involved in histidine biosynthesis. In another embodiment, the metabolic enzyme is encoded by hisD, involved in histidine biosynthesis. In another embodiment, the metabolic enzyme is encoded by hisG, involved in histidine biosynthesis. In another embodiment, the metabolic enzyme is encoded by metX, involved in methionine biosynthesis. In another embodiment, the metabolic enzyme is encoded by proB, involved in proline biosynthesis. In another embodiment, the metabolic enzyme is encoded by argR, involved in arginine biosynthesis. In another embodiment, the metabolic enzyme is encoded by argJ, involved in arginine biosynthesis. In another embodiment, the metabolic enzyme is encoded by thil, involved in thiamine biosynthesis. In another embodiment, the metabolic enzyme is encoded by LMOf2365_{—}1652, involved in tryptophan biosynthesis. In another embodiment, the metabolic enzyme is encoded by aroA, involved in tryptophan biosynthesis. In another embodiment, the metabolic enzyme is encoded by ilvD, involved in valine and isoleucine biosynthesis. In another embodiment, the metabolic enzyme is encoded by ilvC, involved in valine and isoleucine biosynthesis. In another embodiment, the metabolic enzyme is encoded by leuA, involved in leucine biosynthesis. In another embodiment, the metabolic enzyme is encoded by dapF, involved in lysine biosynthesis. In another embodiment, the metabolic enzyme is encoded by thrB, involved in threonine biosynthesis (all GenBank Accession No. NC_{—}002973).

In another embodiment, the metabolic enzyme is a tRNA synthetase. In another embodiment, the metabolic enzyme is encoded by the trpS gene, encoding tryptophanyl tRNA synthetase. In another embodiment, the metabolic enzyme is any other tRNA synthetase known in the art. Each possibility represents a separate embodiment of the methods and compositions provided herein.

In another embodiment, a recombinant Listeria strain provided herein has been passaged through an animal host. In another embodiment, the passaging maximizes efficacy of the strain as a vaccine vector. In another embodiment, the passaging stabilizes the immunogenicity of the Listeria strain. In another embodiment, the passaging stabilizes the virulence of the Listeria strain. In another embodiment, the passaging increases the immunogenicity of the Listeria strain. In another embodiment, the passaging increases the virulence of the Listeria strain. In another embodiment, the passaging removes unstable sub-strains of the Listeria strain. In another embodiment, the passaging reduces the prevalence of unstable sub-strains of the Listeria strain. In another embodiment, the passaging attenuates the strain, or in another embodiment, makes the strain less virulent. Methods for passaging a recombinant Listeria strain through an animal host are well known in the art, and are described, for example, in U.S. patent application Ser. No. 10/541,614. Each possibility represents a separate embodiment of the methods and composition provided herein.

The recombinant Listeria strain of the methods and compositions provided herein is, in another embodiment, a recombinant Listeria monocytogenes strain. In another embodiment, the Listeria strain is a recombinant Listeria seeligeri strain. In another embodiment, the Listeria strain is a recombinant Listeria grayi strain. In another embodiment, the Listeria strain is a recombinant Listeria ivanovii strain. In another embodiment, the Listeria strain is a recombinant Listeria murrayi strain. In another embodiment, the Listeria strain is a recombinant Listeria welshimeri strain. In another embodiment, the Listeria strain is a recombinant strain of any other Listeria species known in the art. Each possibility represents a separate embodiment provided herein. In another embodiment, the sequences of Listeria proteins for use in the methods and compositions provided herein are from any of the above-described strains.

In one embodiment, a Listeria monocytogenes strain provided herein is the EGD strain, the 10403S strain, the NICPBP 54002 strain, the S3 strain, the NCTC 5348 strain, the NICPBP 54006 strain, the M7 strain, the S19 strain, or another strain of Listeria monocytogenes which is known in the art.

In another embodiment, the recombinant Listeria strain is a vaccine strain, which in one embodiment, is a bacterial vaccine strain.

In another embodiment, the present invention provides an immunogenic composition comprising a recombinant Listeria of the present invention. In another embodiment, the immunogenic composition of methods and compositions of the present invention comprises a recombinant vaccine vector of the present invention. In another embodiment, the immunogenic composition comprises a plasmid of the present invention. In another embodiment, the immunogenic composition comprises an adjuvant. In one embodiment, a vector of the present invention may be administered as part of a vaccine composition. Each possibility represents a separate embodiment of the present invention.

In another embodiment, a vaccine of the present invention is delivered with an adjuvant. In one embodiment, the adjuvant favors a predominantly Th1-mediated immune response. In another embodiment, the adjuvant favors a Th1-type immune response. In another embodiment, the adjuvant favors a Th1-mediated immune response. In another embodiment, the adjuvant favors a cell-mediated immune response over an antibody-mediated response. In another embodiment, the adjuvant is any other type of adjuvant known in the art. In another embodiment, the immunogenic composition induces the formation of a T cell immune response against the target protein.

In another embodiment, the adjuvant is MPL. In another embodiment, the adjuvant is QS21. In another embodiment, the adjuvant is a TLR agonist. In another embodiment, the adjuvant is a TLR4 agonist. In another embodiment, the adjuvant is a TLR9 agonist. In another embodiment, the adjuvant is Resiquimod®. In another embodiment, the adjuvant is imiquimod. In another embodiment, the adjuvant is a CpG oligonucleotide. In another embodiment, the adjuvant is a cytokine or a nucleic acid encoding same. In another embodiment, the adjuvant is a chemokine or a nucleic acid encoding same. In another embodiment, the adjuvant is IL-12 or a nucleic acid encoding same. In another embodiment, the adjuvant is IL-6 or a nucleic acid encoding same. In another embodiment, the adjuvant is a lipopolysaccharide. In another embodiment, the adjuvant is as described in Fundamental Immunology, 5th ed (August 2003): William E. Paul (Editor); Lippincott Williams & Wilkins Publishers; Chapter 43: Vaccines, GJV Nossal, which is hereby incorporated by reference. In another embodiment, the adjuvant is any other adjuvant known in the art. Each possibility represents a separate embodiment of the methods and composition provided herein.

In one embodiment, a method of present invention further comprises the step of boosting the human subject with a recombinant Listeria strain provided herein. In another embodiment, the recombinant strain used in the booster inoculation is the same as the strain used in the initial “priming” inoculation. In another embodiment, the booster strain is different from the priming strain. In another embodiment, the same doses are used in the priming and boosting inoculations. In another embodiment, a larger dose is used in the booster. In another embodiment, a smaller dose is used in the booster. Each possibility represents a separate embodiment of the methods and composition provided herein.

In one embodiment, the first, second or third nucleic acid molecule encodes a prostate specific antigen (PSA) and the method is for treating, inhibiting or suppressing prostate cancer. In another embodiment, the first, second or third nucleic acid molecule encodes PSA and the method is for treating, inhibiting or suppressing ovarian cancer. In another embodiment, the first, second or third nucleic acid molecule encodes PSA and the method is treating, inhibiting, or suppressing metastasis of prostate cancer, which in one embodiment, comprises metastasis to bone, and in another embodiment, comprises metastasis to other organs. In another embodiment, the first, second or third nucleic acid molecule encodes PSA and the method is for treating, inhibiting or suppressing metastasis of prostate cancer to bones. In yet another embodiment the method is for treating, inhibiting, or suppressing metastasis of prostate cancer to other organs. In another embodiment, the first, second or third nucleic acid molecule encodes PSA and the method is for treating, inhibiting or suppressing breast cancer. In another embodiment, the first, second or third nucleic acid molecule encodes PSA and the method is for treating, inhibiting or suppressing both ovarian and breast cancer.

In one embodiment, the first, second or third nucleic acid molecule encodes a High Molecular Weight-Melanoma Associated Antigen (HMW-MAA) and the method is for treating, inhibiting or suppressing melanoma. In another embodiment, the first, second or third nucleic acid molecule encodes HMW-MAA and the method is for treating, inhibiting or suppressing breast cancer. In another embodiment, the first, second or third nucleic acid molecule encodes HMW-MAA and the method is for treating, inhibiting or suppressing ovarian cancer. In another embodiment, the first, second or third nucleic acid molecule encodes HMW-MAA and the method is for treating, inhibiting or suppressing benign nevi lesions. In another embodiment, the first, second or third nucleic acid molecule encodes HMW-MAA and the method is for treating, inhibiting or suppressing basal cell carcinoma. In another embodiment, the first, second or third nucleic acid molecule encodes HMW-MAA and the method is for treating, inhibiting or suppressing a tumor of neural crest origin, which in one embodiment, is an astrocytoma, glioma, neuroblastoma, sarcoma, or combination thereof. In another embodiment, the first, second or third nucleic acid molecule encodes HMW-MAA and the method is for treating, inhibiting or suppressing a childhood leukemia, which in one embodiment, is Childhood Acute Lymphoblastic Leukemia, and in another embodiment, is Childhood Acute Myeloid Leukemia (which in one embodiment, is acute myelogenous leukemia, acute myeloid leukemia, acute myelocytic leukemia, or acute non-lymphocytic leukemia) and in another embodiment, is acute lymphocytic leukemia (which in one embodiment, is called acute lymphoblastic leukemia, and in another embodiment, is acute myelogenous leukemia (also called acute myeloid leukemia, acute myelocytic leukemia, or acute non-lymphocytic leukemia) and in another embodiment, is Hybrid or mixed lineage leukemia. In another embodiment, the first or second polypeptide comprises HMW-MAA and the method is for treating, inhibiting or suppressing Chronic myelogenous leukemia or Juvenile Myelomonocytic Leukemia (JMML). In another embodiment, the first, second or third nucleic acid molecule encodes HMW-MAA and the method is for treating, inhibiting or suppressing lobular breast carcinoma lesions.

The cancer that is the target of methods and compositions provided herein is, in another embodiment, a melanoma. In another embodiment, the cancer is a sarcoma. In another embodiment, the cancer is a carcinoma. In another embodiment, the cancer is a mesothelioma (e.g. malignant mesothelioma). In another embodiment, the cancer is a glioma. In another embodiment, the cancer is a germ cell tumor. In another embodiment, the cancer is a choriocarcinoma.

In another embodiment, the cancer is pancreatic cancer. In another embodiment, the cancer is ovarian cancer. In another embodiment, the cancer is gastric cancer. In another embodiment, the cancer is a carcinomatous lesion of the pancreas. In another embodiment, the cancer is pulmonary adenocarcinoma. In another embodiment, the cancer is colorectal adenocarcinoma. In another embodiment, the cancer is pulmonary squamous adenocarcinoma. In another embodiment, the cancer is gastric adenocarcinoma. In another embodiment, the cancer is an ovarian surface epithelial neoplasm (e.g. a benign, proliferative or malignant variety thereof). In another embodiment, the cancer is an oral squamous cell carcinoma. In another embodiment, the cancer is non small-cell lung carcinoma. In another embodiment, the cancer is an endometrial carcinoma. In another embodiment, the cancer is a bladder cancer. In another embodiment, the cancer is a head and neck cancer. In another embodiment, the cancer is a prostate carcinoma.

In another embodiment, the cancer is a non-small cell lung cancer (NSCLC). In another embodiment, the cancer is a colon cancer. In another embodiment, the cancer is a lung cancer. In another embodiment, the cancer is an ovarian cancer. In another embodiment, the cancer is a uterine cancer. In another embodiment, the cancer is a thyroid cancer. In another embodiment, the cancer is a hepatocellular carcinoma. In another embodiment, the cancer is a thyroid cancer. In another embodiment, the cancer is a liver cancer. In another embodiment, the cancer is a renal cancer. In another embodiment, the cancer is a kaposis. In another embodiment, the cancer is a sarcoma. In another embodiment, the cancer is another carcinoma or sarcoma. Each possibility represents a separate embodiment of the methods and composition provided herein.

In one embodiment, the compositions and methods provided herein are used to treat solid tumors related to or resulting from any of the cancers described hereinabove. In another embodiment, the tumor is a Wilms' tumor. In another embodiment, the tumor is a desmoplastic small round cell tumor.

Methods for assessing efficacy of prostate cancer vaccines are well known in the art, and are described, for example, in Dzojic H et al (Adenovirus-mediated CD40 ligand therapy induces tumor cell apoptosis and systemic immunity in the TRAMP-C2 mouse prostate cancer model. Prostate. 2006 Jun. 1; 66(8):831-8), Naruishi K et al (Adenoviral vector-mediated RTVP-1 gene-modified tumor cell-based vaccine suppresses the development of experimental prostate cancer. Cancer Gene Ther. 2006 July; 13(7):658-63), Sehgal I et al (Cancer Cell Int. 2006 Aug. 23; 6:21), and Heinrich J E et al (Vaccination against prostate cancer using a live tissue factor deficient cell line in Lobund-Wistar rats. Cancer Immunol Immunother 2007; 56 (5):725-30). Each possibility represents a separate embodiment provided herein.

In another embodiment, the prostate cancer model used to test methods and compositions provided herein is the TPSA23 (derived from TRAMP-C1 cell line stably expressing PSA) mouse model. In another embodiment, the prostate cancer model is a 178-2 BMA cell model. In another embodiment, the prostate cancer model is a PAIII adenocarcinoma cells model. In another embodiment, the prostate cancer model is a PC-3M model. In another embodiment, the prostate cancer model is any other prostate cancer model known in the art. Each possibility represents a separate embodiment of the methods and composition provided herein.

In another embodiment, the vaccine is tested in human subjects, and efficacy is monitored using methods well known in the art, e.g. directly measuring CD4⁺ and CD8⁺ T cell responses, or measuring disease progression, e.g. by determining the number or size of tumor metastases, or monitoring disease symptoms (cough, chest pain, weight loss, etc). Methods for assessing the efficacy of a prostate cancer vaccine in human subjects are well known in the art, and are described, for example, in Uenaka A et al (T cell immunomonitoring and tumor responses in patients immunized with a complex of cholesterol-bearing hydrophobized pullulan (CHP) and NY-ESO-1 protein. Cancer Immun 2007 Apr. 19; 7:9) and Thomas-Kaskel A K et al (Vaccination of advanced prostate cancer patients with PSCA and PSA peptide-loaded dendritic cells induces DTH responses that correlate with superior overall survival. Int J. Cancer. 2006 Nov. 15; 119(10):2428-34). Each method represents a separate embodiment of the methods and composition provided herein.

In another embodiment, the present invention provides a method of treating benign prostate hyperplasia (BPH) in a subject. In another embodiment, the present invention provides a method of treating Prostatic Intraepithelial Neoplasia (PIN) in a subject.

Further, in another embodiment, the compositions or vaccines are administered as a suppository, for example a rectal suppository or a urethral suppository. Further, in another embodiment, the pharmaceutical compositions are administered by subcutaneous implantation of a pellet. In a further embodiment, the pellet provides for controlled release of an agent over a period of time. In yet another embodiment, the pharmaceutical compositions are administered in the form of a capsule.

In one embodiment, the route of administration may be parenteral. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof. For intranasal administration or application by inhalation, solutions or suspensions of the compounds mixed and aerosolized or nebulized in the presence of the appropriate carrier suitable. Such an aerosol may comprise any agent described herein. In one embodiment, the compositions as set forth herein may be in a form suitable for intracranial administration, which in one embodiment, is intrathecal and intracerebroventricular administration. In one embodiment, the regimen of administration will be determined by skilled clinicians, based on factors such as exact nature of the condition being treated, the severity of the condition, the age and general physical condition of the patient, body weight, and response of the individual patient, etc.

In one embodiment, parenteral application, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

Sustained or directed release compositions can be formulated, e.g., liposomes or those wherein the active compound is protected with differentially degradable coatings, e.g., by microencapsulation, multiple coatings, etc. Such compositions may be formulated for immediate or slow release. It is also possible to freeze-dry the new compounds and use the lyophilisates obtained, for example, for the preparation of products for injection.

In one embodiment, for liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In one embodiment, compositions of this invention are pharmaceutically acceptable. In one embodiment, the term “pharmaceutically acceptable” refers to any formulation which is safe, and provides the appropriate delivery for the desired route of administration of an effective amount of at least one compound for use in the present invention. This term refers to the use of buffered formulations as well, wherein the pH is maintained at a particular desired value, ranging from pH 4.0 to pH 9.0, in accordance with the stability of the compounds and route of administration.

In one embodiment, a composition of or used in the methods of this invention may be administered alone or within a composition. In another embodiment, compositions of this invention admixture with conventional excipients, i.e., pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In one embodiment, the compositions for use in the methods and compositions provided herein may be administered with a carrier/diluent. Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In one embodiment, the compositions of the methods and composition provided herein may comprise the composition of this invention and one or more additional compounds effective in preventing or treating cancer. In some embodiments, the additional compound may comprise a compound useful in chemotherapy, which in one embodiment, is Cisplatin. In another embodiment, Ifosfamide, Fluorouracilor5-FU, Irinotecan, Paclitaxel (Taxol), Docetaxel, Gemcitabine, Topotecan or a combination thereof, may be administered with a composition provided herein for use in the methods provided herein. In another embodiment, Amsacrine, Bleomycin, Busulfan, Capecitabine, Carboplatin, Carmustine, Chlorambucil, Cisplatin, Cladribine, Clofarabine, Crisantaspase, Cyclophosphamide, Cytarabine, Dacarbazine, Dactinomycin, Daunorubicin, Docetaxel, Doxorubicin, Epirubicin, Etoposide, Fludarabine, Fluorouracil, Gemcitabine, Gliadelimplants, Hydroxycarbamide, Idarubicin, Ifosfamide, Irinotecan, Leucovorin, Liposomaldoxorubicin, Liposomaldaunorubicin, Lomustine, Melphalan, Mercaptopurine, Mesna, Methotrexate, Mitomycin, Mitoxantrone, Oxaliplatin, Paclitaxel, Pemetrexed, Pentostatin, Procarbazine, Raltitrexed, Satraplatin, Streptozocin, Tegafur-uracil, Temozolomide, Teniposide, Thiotepa, Tioguanine, Topotecan, Treosulfan, Vinblastine, Vincristine, Vindesine, Vinorelbine, or a combination thereof, may be administered with a composition provided herein for use in the methods provided herein.

In one embodiment, provided herein is a recombinant Listeria capable of expressing and secreting at least three distinct heterologous antigens comprising a first antigen that is operably integrated in the genome as an open reading frame with a first polypeptide or fragment thereof comprising a PEST sequence, a second and a third antigen that are genetically fused in an episomal plasmid vector each to a PEST sequence-containing polypeptide. In another embodiment, the first or second polypeptide or fragment thereof is ActA, or LLO. In another embodiment, the first or second antigen is prostate tumor-associated antigen (PSA), or High Molecular Weight-Melanoma Associated Antigen (HMWMAA). In another embodiment, the fragment is an immunogenic fragment. In yet another embodiment, the episomal expression vector lacks an antibiotic resistance marker.

In one embodiment, provided herein is a method of preparing a recombinant Listeria capable of expressing and secreting at least two distinct heterologous antigens that target tumor cells and angiogenesis concomitantly. In another embodiment, the method of preparing the recombinant Listeria comprises the steps of transforming the recombinant Listeria with an episomal recombinant nucleic acid encoding the at least two antigens each fused to a PEST-containing gene.

In another embodiment, the first and at least second antigen are distinct. In another embodiment, the first and at least second antigens are concomitantly expressed. In another embodiment, the first or at least second antigen are expressed at the same level. In another embodiment, the first or at least second antigen are differentially expressed. In another embodiment, gene or protein expression is determined by methods that are well known in the art which in another embodiment comprise real-time PCR, northern blotting, immunoblotting, etc. In another embodiment, the first or at least second antigen's expression is controlled by an inducible system, while in another embodiment, the first or at least second antigen's expression is controlled by a constitutive promoter. In another embodiment, inducible expression systems are well known in the art.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, D.C.; Miller, 1992, A Short Course in Bacterial Genetics, Cold Spring Harbor Laboratory Press, Cold Spring Harbor, N.Y.) In another embodiment, the Listeria vaccine strain provided herein is transformed by electroporation. Each method represents a separate embodiment of the methods and compositions provided herein.

In one embodiment, the present invention provides a method of producing a recombinant Listeria strain expressing at least two antigens, the method comprising: (a) genetically fusing a first nucleic acid encoding a first antigen into the Listeria genome in an open reading frame with an endogenous PEST-containing gene; (b) transforming the recombinant Listeria with an episomal expression vector comprising at least a second nucleic acid encoding at least a second antigen; and (c) expressing the first and the at least second antigens under conditions conducive to antigenic expression in the recombinant Listeria strain.

In one embodiment, the present invention provides a method of producing a recombinant Listeria strain expressing at least three antigens, the method comprising: (a) genetically fusing a first nucleic acid encoding a first antigen into the Listeria genome in an open reading frame with an endogenous PEST-containing polypeptide; (b) transforming the recombinant Listeria with an episomal expression vector comprising a second and a third nucleic acid encoding a second and a third antigen; and (c) expressing the first, second and third antigens under conditions conducive to antigenic expression in the recombinant Listeria strain.

In one embodiment, “antigen” is used herein to refer to a substance that when placed in contact with an organism, results in a detectable immune response from the organism. An antigen may be a lipid, peptide, protein, carbohydrate, nucleic acid, or combinations and variations thereof.

In one embodiment, “variant” refers to an amino acid or nucleic acid sequence (or in other embodiments, an organism or tissue) that is different from the majority of the population but is still sufficiently similar to the common mode to be considered to be one of them, for example splice variants.

In one embodiment, “isoform” refers to a version of a molecule, for example, a protein, with only slight differences compared to another isoform, or version, of the same protein. In one embodiment, isoforms may be produced from different but related genes, or in another embodiment, may arise from the same gene by alternative splicing. In another embodiment, isoforms are caused by single nucleotide polymorphisms.

In one embodiment, “fragment” refers to a protein or polypeptide that is shorter or comprises fewer amino acids than the full length protein or polypeptide. In another embodiment, fragment refers to a nucleic acid that is shorter or comprises fewer nucleotides than the full length nucleic acid. In another embodiment, the fragment is an N-terminal fragment. In another embodiment, the fragment is a C-terminal fragment. In one embodiment, the fragment is an intrasequential section of the protein, peptide, or nucleic acid. In one embodiment, the fragment is a functional fragment. In another embodiment, the fragment is an immunogenic fragment. In one embodiment, a fragment has 10-20 nucleic or amino acids, while in another embodiment, a fragment has more than 5 nucleic or amino acids, while in another embodiment, a fragment has 100-200 nucleic or amino acids, while in another embodiment, a fragment has 100-500 nucleic or amino acids, while in another embodiment, a fragment has 50-200 nucleic or amino acids, while in another embodiment, a fragment has 10-250 nucleic or amino acids.

In one embodiment, “immunogenicity” or “immunogenic” refers to the innate ability of a protein, peptide, nucleic acid, antigen or organism to elicit an immune response in an animal when the protein, peptide, nucleic acid, antigen or organism is administered to the animal. Thus, “enhancing the immunogenicity” in one embodiment, refers to increasing the ability of a protein, peptide, nucleic acid, antigen or organism to elicit an immune response in an animal when the protein, peptide, nucleic acid, antigen or organism is administered to an animal. The increased ability of a protein, peptide, nucleic acid, antigen or organism to elicit an immune response can be measured by, in one embodiment, a greater number of antibodies to a protein, peptide, nucleic acid, antigen or organism, a greater diversity of antibodies to an antigen or organism, a greater number of T-cells specific for a protein, peptide, nucleic acid, antigen or organism, a greater cytotoxic or helper T-cell response to a protein, peptide, nucleic acid, antigen or organism, and the like.

In one embodiment, a “homologue” refers to a nucleic acid or amino acid sequence which shares a certain percentage of sequence identity with a particular nucleic acid or amino acid sequence. In one embodiment, a sequence useful in the composition and methods provided herein may be a homologue of a particular LLO sequence or N-terminal fragment thereof, ActA sequence or N-terminal fragment thereof, or PEST sequence described herein or known in the art. In one embodiment, such a homolog maintains In another embodiment, a sequence useful in the composition and methods provided herein may be a homologue of an antigenic polypeptide, or a functional fragment thereof provided herein. In one embodiment, a homolog of a polypeptide and, in one embodiment, the nucleic acid encoding such a homolog, of the present invention maintains the functional characteristics of the parent polypeptide. For example, in one embodiment, a homolog of an antigenic polypeptide of the present invention maintains the antigenic characteristic of the parent polypeptide. In another embodiment, a sequence useful in the composition and methods provided herein may be a homologue of any sequence described herein. In one embodiment, a homologue shares at least 70% identity with a particular sequence. In another embodiment, a homologue shares at least 72% identity with a particular sequence. In another embodiment, a homologue shares at least 75% identity with a particular sequence. In another embodiment, a homologue shares at least 78% identity with a particular sequence. In another embodiment, a homologue shares at least 80% identity with a particular sequence. In another embodiment, a homologue shares at least 82% identity with a particular sequence. In another embodiment, a homologue shares at least 83% identity with a particular sequence. In another embodiment, a homologue shares at least 85% identity with a particular sequence. In another embodiment, a homologue shares at least 87% identity with a particular sequence. In another embodiment, a homologue shares at least 88% identity with a particular sequence. In another embodiment, a homologue shares at least 90% identity with a particular sequence. In another embodiment, a homologue shares at least 92% identity with a particular sequence. In another embodiment, a homologue shares at least 93% identity with a particular sequence. In another embodiment, a homologue shares at least 95% identity with a particular sequence. In another embodiment, a homologue shares at least 96% identity with a particular sequence. In another embodiment, a homologue shares at least 97% identity with a particular sequence. In another embodiment, a homologue shares at least 98% identity with a particular sequence. In another embodiment, a homologue shares at least 99% identity with a particular sequence. In another embodiment, a homologue shares 100% identity with a particular sequence. Each possibility represents a separate embodiment provided herein.

In one embodiment, it is to be understood that a homolog of any of the sequences provided herein and/or as described herein is considered to be a part of the invention.

In one embodiment, “functional” refers to the innate ability of a protein, peptide, nucleic acid, fragment or a variant thereof to exhibit a biological activity or function. In one embodiment, such a biological function is its binding property to an interaction partner, e.g., a membrane-associated receptor, and in another embodiment, its trimerization property. In the case of functional fragments and the functional variants of the invention, these biological functions may in fact be changed, e.g., with respect to their specificity or selectivity, but with retention of the basic biological function.

In one embodiment, “treating” refers to both therapeutic treatment and prophylactic or preventative measures, wherein the object is to prevent or lessen the targeted pathologic condition or disorder as described herein. Thus, in one embodiment, treating may include directly affecting or curing, suppressing, inhibiting, preventing, reducing the severity of, delaying the onset of, reducing symptoms associated with the disease, disorder or condition, or a combination thereof. Thus, in one embodiment, “treating” refers inter alia to delaying progression, expediting remission, inducing remission, augmenting remission, speeding recovery, increasing efficacy of or decreasing resistance to alternative therapeutics, or a combination thereof. In one embodiment, “preventing” or “impeding” refers, inter alia, to delaying the onset of symptoms, preventing relapse to a disease, decreasing the number or frequency of relapse episodes, increasing latency between symptomatic episodes, or a combination thereof. In one embodiment, “suppressing” or “inhibiting”, refers inter alia to reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In one embodiment, symptoms are primary, while in another embodiment, symptoms are secondary. In one embodiment, “primary” refers to a symptom that is a direct result of a particular disease or disorder, while in one embodiment, “secondary” refers to a symptom that is derived from or consequent to a primary cause. In one embodiment, the compounds for use in the present invention treat primary or secondary symptoms or secondary complications. In another embodiment, “symptoms” may be any manifestation of a disease or pathological condition.

In one embodiment, the term “comprising” refers to the inclusion of other recombinant polypeptides, amino acid sequences, or nucleic acid sequences, as well as inclusion of other polypeptides, amino acid sequences, or nucleic acid sequences, that may be known in the art, which in one embodiment may comprise antigens or Listeria polypeptides, amino acid sequences, or nucleic acid sequences. In another embodiments, the term “consisting essentially of” refers to a composition for use in the methods provided herein, which has the specific recombinant polypeptide, amino acid sequence, or nucleic acid sequence, or fragment thereof. However, other polypeptides, amino acid sequences, or nucleic acid sequences may be included that are not involved directly in the utility of the recombinant polypeptide(s). In another embodiment, the term “consisting” refers to a composition for use in the methods provided herein having a particular recombinant polypeptide, amino acid sequence, or nucleic acid sequence, or fragment or combination of recombinant polypeptides, amino acid sequences, or nucleic acid sequences or fragments provided herein, in any form or embodiment as described herein.

In another embodiment of the methods and compositions provided herein, “nucleic acids” or “nucleotide” refers to a string of at least two base-sugar-phosphate combinations. The term includes, in one embodiment, DNA and RNA. “Nucleotides” refers, in one embodiment, to the monomeric units of nucleic acid polymers. RNA may be, in one embodiment, in the form of a tRNA (transfer RNA), snRNA (small nuclear RNA), rRNA (ribosomal RNA), mRNA (messenger RNA), anti-sense RNA, small inhibitory RNA (siRNA), micro RNA (miRNA) and ribozymes. The use of siRNA and miRNA has been described (Caudy A A et al, Genes & Devel 16: 2491-96 and references cited therein). DNA may be in form of plasmid DNA, viral DNA, linear DNA, or chromosomal DNA or derivatives of these groups. In addition, these forms of DNA and RNA may be single, double, triple, or quadruple stranded. The term also includes, in another embodiment, artificial nucleic acids that may contain other types of backbones but the same bases. In one embodiment, the artificial nucleic acid is a PNA (peptide nucleic acid). PNA contain peptide backbones and nucleotide bases and are able to bind, in one embodiment, to both DNA and RNA molecules. In another embodiment, the nucleotide is oxetane modified. In another embodiment, the nucleotide is modified by replacement of one or more phosphodiester bonds with a phosphorothioate bond. In another embodiment, the artificial nucleic acid contains any other variant of the phosphate backbone of native nucleic acids known in the art. The use of phosphothiorate nucleic acids and PNA are known to those skilled in the art, and are described in, for example, Neilsen P E, Curr Opin Struct Biol 9:353-57; and Raz N K et al Biochem Biophys Res Commun 297:1075-84. The production and use of nucleic acids is known to those skilled in art and is described, for example, in Molecular Cloning, (2001), Sambrook and Russell, eds. and Methods in Enzymology: Methods for molecular cloning in eukaryotic cells (2003) Purchio and G. C. Fareed. Each nucleic acid derivative represents a separate embodiment provided herein.

The terms “polypeptide,” “peptide” and “recombinant peptide” refer, in another embodiment, to a peptide or polypeptide of any length. In another embodiment, a peptide or recombinant peptide provided herein has one of the lengths enumerated above for an HMW-MAA fragment. Each possibility represents a separate embodiment of the methods and composition provided herein. In one embodiment, the term “peptide” refers to native peptides (either degradation products, synthetically synthesized peptides or recombinant peptides) and/or peptidomimetics (typically, synthetically synthesized peptides), such as peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

In one embodiment, “antigenic polypeptide” is used herein to refer to a polypeptide, peptide or recombinant peptide as described hereinabove that is foreign to a host and leads to the mounting of an immune response when present in, or, in another embodiment, detected by, the host.

“Stably maintained” refers, in another embodiment, to maintenance of a nucleic acid molecule or plasmid in the absence of selection (e.g. antibiotic selection) for 10 generations, without detectable loss. In another embodiment, the period is 15 generations. In another embodiment, the period is 20 generations. In another embodiment, the period is 25 generations. In another embodiment, the period is 30 generations. In another embodiment, the period is 40 generations. In another embodiment, the period is 50 generations. In another embodiment, the period is 60 generations. In another embodiment, the period is 80 generations. In another embodiment, the period is 100 generations. In another embodiment, the period is 150 generations. In another embodiment, the period is 200 generations. In another embodiment, the period is 300 generations. In another embodiment, the period is 500 generations. In another embodiment, the period is more than 500 generations. In another embodiment, the nucleic acid molecule or plasmid is maintained stably in vitro (e.g. in culture). In another embodiment, the nucleic acid molecule or plasmid is maintained stably in vivo. In another embodiment, the nucleic acid molecule or plasmid is maintained stably both in vitro and in vitro. Each possibility represents a separate embodiment of the methods and compositions provided herein.

In one embodiment, the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” may include both D- and L-amino acids.

The term “nucleic acid” or “nucleic acid sequence” refers to a deoxyribonucleotide or ribonucleotide oligonucleotide in either single- or double-stranded form. The term encompasses nucleic acids, i.e., oligonucleotides, containing known analogues of natural nucleotides which have similar or improved binding properties, for the purposes desired, as the reference nucleic acid. The term also includes nucleic acids which are metabolized in a manner similar to naturally occurring nucleotides or at rates that are improved thereover for the purposes desired. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotries ter, sulfamate, 3′-thio acetal, methylene(methylimino), 3′-N-carbamate, morpholino carbamate, and peptide nucleic acids (PNAs); see, e.g., Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Mulligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., in WO 97/03211; WO 96/39154; Mata (1997) Toxicol. Appl. Pharmacol. 144:189-197. Other synthetic backbones encompasses by the term include methyl-phosphonate linkages or alternating methylphosphonate and phosphodiester linkages (Strauss-Soukup (1997) Biochemistry 36:8692-8698), and benzylphosphonate linkages (S amstag (1996) Anti sense Nucleic Acid Drug Dev. 6:153-156). The term nucleic acid is used interchangeably with gene, cDNA, mRNA, oligonucleotide primer, probe and amplification product.

In one embodiment of the methods and compositions provided herein, the term “recombination site” or “site-specific recombination site” refers to a sequence of bases in a nucleic acid molecule that is recognized by a recombinase (along with associated proteins, in some cases) that mediates exchange or excision of the nucleic acid segments flanking the recombination sites. The recombinases and associated proteins are collectively referred to as “recombination proteins” see, e.g., Landy, A., (Current Opinion in Genetics & Development) 3:699-707; 1993).

A “phage expression vector” or “phagemid” refers to any phage-based recombinant expression system for the purpose of expressing a nucleic acid sequence of the methods and compositions provided herein in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

In one embodiment, the term “operably linked” as used herein means that the transcriptional and translational regulatory nucleic acid, is positioned relative to any coding sequences in such a manner that transcription is initiated. Generally, this will mean that the promoter and transcriptional initiation or start sequences are positioned 5′ to the coding region.

In one embodiment, a “regulator gene” is a gene that encodes a protein that controls the rate of synthesis of another gene. An example of a regulator gene is a gene that encodes a repressor.

In another embodiment, a “repressor” is a protein that is synthesized by a regulator gene and binds to an operator locus, blocking transcription of that operon.

In one embodiment, an “inducer” is a small organic molecule that causes a regulated control sequence to become active.

In one embodiment, “trans regulatory element” refers to a molecule or complex that modulates the expression of a gene. Examples include repressors that bind to operators in a control sequence, activators that cause transcription initiation, and antisense RNA that binds to and prevents translation of an mRNA. In another embodiment, such elements are contemplated for use in the present invention, particularly and as a non-limiting example, when expression of an excessive amount of heterologous antigens present a metabolic burden on the Listeria host which would require regulating plasmid copy number and resultant expression of the heterologous antigens to allow optimal survival of the Listeria vaccine strains and also allow optimal efficiency in inducing the desired immune responses in a subject to which the Listerial vaccine strain has been administered.

Another type of trans regulatory element is RNA polymerase. Plasmid genes encoding heterologous antigens can be regulated by linking them to promoters recognized only by specific RNA polymerases. By regulating the expression of the specific RNA polymerase, expression of the gene is also regulated. For example T7 RNA polymerase requires a specific promoter sequence that is not recognized by bacterial RNA polymerases. A T7 RNA polymerase gene can be placed in the host cell and regulated to be expressed only in the permissive or non-permissive environment. Expression of the T7 RNA polymerase will in turn express any gene linked to a T7 RNA polymerase promoter. A description of how to use T7 RNA polymerase to regulate expression of a gene of interest, including descriptions of nucleic acid sequences useful for this regulation appears in Studier et al., Methods Enzymol. 185:60-89 (1990).

Another type of trans regulatory element is antisense RNA. Antisense RNA is complementary to a nucleic acid sequence, referred to as a target sequence of a gene to be regulated. Hybridization between the antisense RNA and the target sequence prevents expression of the gene. Typically, antisense RNA complementary to the mRNA of a gene is used and the primary effect is to prevent translation of the mRNA. Expression of the genes of a RADS can be regulated by controlling the expression of the antisense RNA. Expression of the antisense RNA in turn prevents expression of the gene of interest, which in the present invention can be any of the heterologous antigens encoded by the nucleic acid molecules provided herein. A complete description of how to use antisense RNA to regulate expression of a gene of interest appears in U.S. Pat. No. 5,190,931, which is incorporated by reference in its entirety herein.

In one embodiment, an “open reading frame” or “ORF” is a portion of an organism's genome which contains a sequence of bases that could potentially encode a protein. In another embodiment, the start and stop ends of the ORF are not equivalent to the ends of the mRNA, but they are usually contained within the mRNA. In one embodiment, ORFs are located between the start-code sequence (initiation codon) and the stop-codon sequence (termination codon) of a gene. Thus, in one embodiment, a nucleic acid molecule operably integrated into a genome as an open reading frame with an endogenous polypeptide is a nucleic acid molecule that has integrated into a genome in the same open reading frame as an endogenous polypeptide.

In one embodiment, the present invention provides a fusion polypeptide comprising a linker sequence. In one embodiment, a “linker sequence” refers to an amino acid sequence that joins two heterologous polypeptides, or fragments or domains thereof. In general, as used herein, a linker is an amino acid sequence that covalently links the polypeptides to form a fusion polypeptide. A linker typically includes the amino acids translated from the remaining recombination signal after removal of a reporter gene from a display vector to create a fusion protein comprising an amino acid sequence encoded by an open reading frame and the display protein. As appreciated by one of skill in the art, the linker can comprise additional amino acids, such as glycine and other small neutral amino acids.

In one embodiment, the terms “episomal expression vector”, or “episomal recombinant nucleic acid” refer to a nucleic acid vector which may be linear or circular, and which is usually double-stranded in form. In one embodiment, an episomal expression vector comprises a gene of interest. In another embodiment, the inserted gene of interest is not interrupted or subjected to regulatory constraints which often occur from integration into cellular DNA. In another embodiment, the presence of the inserted heterologous gene does not lead to rearrangement or interruption of the cell's own important regions. In another embodiment, episomal vectors persist in multiple copies in the bacterial cytoplasm, resulting in amplification of the gene of interest, and, in another embodiment, viral trans-acting factors are supplied when necessary. In another embodiment, in stable transfection procedures, the use of episomal vectors often results in higher transfection efficiency than the use of chromosome-integrating plasmids (Belt, P. B. G. M., et al (1991) Efficient cDNA cloning by direct phenotypic correction of a mutant human cell line (HPRT2) using an Epstein-Barr virus-derived cDNA expression vector. Nucleic Acids Res. 19, 4861-4866; Mazda, O., et al. (1997) Extremely efficient gene transfection into lympho-hematopoietic cell lines by Epstein-Barr virus-based vectors. J. Immunol. Methods 204, 143-151). In one embodiment, the episomal expression vectors of the methods and compositions provided herein may be delivered to cells in vivo, ex vivo, or in vitro by any of a variety of the methods employed to deliver DNA molecules to cells. The vectors may also be delivered alone or in the form of a pharmaceutical composition that enhances delivery to cells of a subject.

In another embodiment, conjugation is used to introduce genetic material and/or plasmids into bacteria. Methods for conjugation are well known in the art, and are described, for example, in Nikodinovic J et al (A second generation snp-derived Escherichia coli-Streptomyces shuttle expression vector that is generally transferable by conjugation. Plasmid. 2006 November; 56(3):223-7) and Auchtung J M et al (Regulation of a Bacillus subtilis mobile genetic element by intercellular signaling and the global DNA damage response. Proc Natl Acad Sci USA. 2005 Aug. 30; 102(35):12554-9). Each method represents a separate embodiment of the methods and compositions provided herein.

“Metabolic enzyme” refers, in another embodiment, to an enzyme involved in synthesis of a nutrient required by the host bacteria. In another embodiment, the term refers to an enzyme required for synthesis of a nutrient required by the host bacteria. In another embodiment, the term refers to an enzyme involved in synthesis of a nutrient utilized by the host bacteria. In another embodiment, the term refers to an enzyme involved in synthesis of a nutrient required for sustained growth of the host bacteria. In another embodiment, the enzyme is required for synthesis of the nutrient. Each possibility represents a separate embodiment of the methods and compositions provided herein.

In another embodiment, the present invention provides a kit for conveniently practicing the methods provided herein comprising one or more Listeria strains provided herein, an applicator, and instructional material that describes how to use the kit components in practicing the methods provided herein.

In one embodiment, the term “about” refers to in quantitative terms plus or minus 5%, or in another embodiment plus or minus 10%, or in another embodiment plus or minus 15%, or in another embodiment plus or minus 20%.

In one embodiment, the term “subject” refers to a mammal including a human in need of therapy for, or susceptible to, a condition or its sequelae. The subject may include dogs, cats, pigs, cows, sheep, goats, horses, rats, and mice and humans. In one embodiment, the term “subject” does not exclude an individual that is healthy in all respects and does not have or show signs of disease or disorder.

The following examples are presented in order to more fully illustrate the preferred embodiments of the invention. They should in no way be construed, however, as limiting the broad scope of the invention.

### EXAMPLES

A recombinant Lm that secretes PSA fused to tLLO (Lm-LLO-PSA) was developed, which elicits a potent PSA-specific immune response associated with regression of tumors in a mouse model for prostate cancer, wherein the expression of tLLO-PSA is derived from a plasmid based on pGG55 (Table 1), which confers antibiotic resistance to the vector for a strain for the PSA vaccine based on the pADV142 plasmid was also developed. This strain, has no antibiotic resistance markers, and is referred as LmddA-142 (Table 1). This new strain is 10 times more attenuated than Lm-LLO-PSA. In addition, LmddA-142 was slightly more immunogenic and significantly more efficacious in regressing PSA expressing tumors than the Lm-LLO-PSA.

TABLE 1
Plasmids and strains

| Plasmids | Features |
|---|---|
| pGG55 | pAM401/pGB354 shuttle plasmid with gram(−) |
| | and gram(+) cm resistance, LLO-E7 expression |
| | cassette and a copy of LmprfA gene |
| pTV3 | Derived from pGG55 by deleting cm genes and |
| | inserting the Lmdal gene |
| pADV119 | Derived from pTV3 by deleting the prfA gene |
| pADV134 | Derived from pADV119 by replacing the Lmdal |
| | gene by the Bacillusdal gene |
| pADV142 | Derived from pADV134 by replacing HPV16 e7 |
| | with klk3 |
| pADV172 | Derived from pADV134 by replacing HPV16 e7 |
| | with hmw-maa₂₁₆₀₋₂₂₅₈ |
| Strains | Genotype |
| 10403S | Wild-type Listeria monocytogenes:: str |
| XFL-7 | 10403S prfA⁽⁻⁾ |
| Lmdd | 10403S dal⁽⁻⁾ dat⁽⁻⁾ |
| LmddA | 10403S dal⁽⁻⁾ dat⁽⁻⁾ actA⁽⁻⁾ |
| LmddA-134 | 10403S dal⁽⁻⁾ dat⁽⁻⁾ actA⁽⁻⁾ pADV134 |
| LmddA-142 | 10403S dal⁽⁻⁾ dat⁽⁻⁾ actA⁽⁻⁾ pADV142 |
| Lmdd-143 | 10403S dal⁽⁻⁾ dat⁽⁻⁾ with klk3 fused to the |
| | hly gene in the chromosome |
| LmddA-143 | 10403S dal⁽⁻⁾ dat⁽⁻⁾ actA⁽⁻⁾ with klk3 |
| | fused to the hly gene in the chromosome |
| LmddA-172 | 10403S dal⁽⁻⁾ dat⁽⁻⁾ actA⁽⁻⁾ pADV172 |
| Lmdd-143/134 | Lmdd-143 pADV134 |
| LmddA-143/134 | LmddA-143 pADV134 |
| Lmdd-143/172 | Lmdd-143 pADV172 |
| LmddA-143/172 | LmddA-143 pADV172 |

The sequence of the plasmid pAdv142 (6523 bp) was as follows:

cggagtgtatactggcttactatgttggcactgatgagggtgtcagtgaagtgcttcatgtggcaggagaaaaaaggctgc accggtgcgtcagcagaatatgtgatacaggatatattccgcttcctcgctcactgactcgctacgctcggtcgttcgactgcggcgagc ggaaatggcttacgaacggggcggagatttcctggaagatgccaggaagatacttaacagggaagtgagagggccgcggcaaagc cgtttttccataggctccgcccccctgacaagcatcacgaaatctgacgctcaaatcagtggtggcgaaacccgacaggactataaaga taccaggcgtttccccctggcggctccctcgtgcgctctcctgttcctgcctttcggtttaccggtgtcattccgctgttatggccgcgtttgt ctcattccacgcctgacactcagttccgggtaggcagttcgctccaagctggactgtatgcacgaaccccccgttcagtccgaccgctg cgccttatccggtaactatcgtcttgagtccaacccggaaagacatgcaaaagcaccactggcagcagccactggtaattgatttagag gagttagtcttgaagtcatgcgccggttaaggctaaactgaaaggacaagattggtgactgcgctcctccaagccagttacctcggaca aagagaggtagctcagagaaccacgaaaaaccgccctgcaaggcggattacgattcagagcaagagattacgcgcagaccaaaa cgatctcaagaagatcatcttattaatcagataaaatatactagccctcattgattagtatattcctatcttaaagttactatatgtggaggc a ttaacatttgttaatgacgtcaaaaggatagcaagactagaataaagctataaagcaagcatataatattgcgtttcatctttagaagcgaat ttcgccaatattataattatcaaaagagaggggtggcaaacggtataggcattattaggttaaaaaatgtagaaggagagtgaaacccat gaaaaaaataatgctagtattattacacttatattagttagtctaccaattgcgcaacaaactgaagcaaaggatgcatctgcattcaataaa gaaaattcaatttcatccatggcaccaccagcatctccgcctgcaagtcctaagacgccaatcgaaaagaaacacgcggatgaaatcg ataagtatatacaaggattggattacaataaaaacaatgtattagtataccacggagatgcagtgacaaatgtgccgccaagaaaaggtt acaaagatggaaatgaatatattgagtggagaaaaagaagaaatccatcaatcaaaataatgcagacattcaagagtgaatgcaatttc gagcctaacctatccaggtgctctcgtaaaagcgaattcggaattagtagaaaatcaaccagatgactccctgtaaaacgtgattcattaa cactcagcattgatttgccaggtatgactaatcaagacaataaaatagagtaaaaaatgccactaaatcaaacgttaacaacgcagtaaa tacattagtggaaagatggaatgaaaaatatgctcaagcttatccaaatgtaagtgcaaaaattgattatgatgacgaaatggcttacagtg aatcacaattaattgcgaaataggtacagcatttaaagctgtaaataatagcttgaatgtaaacttcggcgcaatcagtgaagggaaaatg caagaagaagtcattagattaaacaaatttactataacgtgaatgttaatgaacctacaagaccaccagattatcggcaaagctgttacta aagagcagagcaagcgcttggagtgaatgcagaaaatcctcctgcatatatctcaagtgtggcgtatggccgtcaagatatttgaaatta tcaactaattcccatagtactaaagtaaaagctgcttttgatgctgccgtaagcggaaaatctgtctcaggtgatgtagaactaacaaatat catcaaaaattcaccacaaagccgtaatttacggaggaccgcaaaagatgaagttcaaatcatcgacggcaacctcggagacttacg cgatattagaaaaaaggcgctacattaatcgagaaacaccaggagacccattgcttatacaacaaacacctaaaagacaatgaattag ctgttattaaaaacaactcagaatatattgaaacaacttcaaaagcttatacagatggaaaaattaacatcgatcactctggaggatacgtt gctcaattcaacatttcagggatgaagtaaattatgatctcgagattgtgggaggctgggagtgcgagaagcattcccaaccctggcag gtgcttgtggcctctcgtggcagggcagtctgcggcggtgactggtgcacccccagtgggtcctcacagctgcccactgcatcagga acaaaagcgtgatcttgctgggtcggcacagcctgtttcatcctgaagacacaggccaggtatttcaggtcagccacagcttcccacac ccgctctacgatatgagcctcctgaagaatcgattcctcaggccaggtgatgactccagccacgacctcatgctgctccgcctgtcaga gcctgccgagctcacggatgctgtgaaggtcatggacctgcccacccaggagccagcactggggaccacctgctacgcctcaggct ggggcagcattgaaccagaggagacttgaccccaaagaaacttcagtgtgtggacctccatgttataccaatgacgtgtgtgcgcaag ttcaccctcagaaggtgaccaagttcatgctgtgtgctggacgctggacagggggcaaaagcacctgctcgggtgattctgggggccc acttgtctgttatggtgtgcttcaaggtatcacgtcatggggcagtgaaccatgtgccctgcccgaaaggccttccctgtacaccaaggtg gtgcattaccggaagtggatcaaggacaccatcgtggccaaccccTAAcccgggccactaactcaacgctagtagtggatttaatcc caaatgagccaacagaaccagaaccagaaacagaacaagtaacattggagttagaaatggaagaagaaaaaagcaatgatttcgtgt gaataatgcacgaaatcattgcttattatttaaaaagcgatatactagatataacgaaacaacgaactgaataaagaatacaaaaaaaga gccacgaccagttaaagcctgagaaactttaactgcgagccttaattgattaccaccaatcaattaaagaagtcgagacccaaaataggt aaagtatttaattactttattaatcagatacttaaatatctgtaaacccattatatcgggtattgaggggatttcaagtattaagaagatacca ggcaatcaattaagaaaaacttagttgattgccattagagtgattcaactagatcgtagatctaactaattaattacgtaagaaaggaga acagctgaatgaatatcccattgagtagaaactgtgcttcatgacggcttgttaaagtacaaatttaaaaatagtaaaattcgctcaatcac taccaagccaggtaaaagtaaaggggctatattgcgtatcgctcaaaaaaaagcatgattggcggacgtggcgttgactgacttccga agaagcgattcacgaaaatcaagatacatttacgcattggacaccaaacgtttatcgttatggtacgtatgcagacgaaaaccgttcatac actaaaggacattctgaaaacaatttaagacaaatcaataccttctttattgattttgatattcacacggaaaaagaaactatttcagcaagc gatattttaacaacagctattgatttaggttttatgcctacgttaattatcaaatctgataaaggttatcaagcatattttgttttagaaacgccag tctatgtgacttcaaaatcagaatttaaatctgtcaaagcagccaaaataatctcgcaaaatatccgagaatattttggaaagtctttgccag ttgatctaacgtgcaatcattttgggattgctcgtataccaagaacggacaatgtagaattttttgatcccaattaccgttattctttcaaagaa tggcaagattggtctttcaaacaaacagataataagggctttactcgttcaagtctaacggttttaagcggtacagaaggcaaaaaacaa gtagatgaaccctggtttaatctcttattgcacgaaacgaaattttcaggagaaaagggtttagtagggcgcaatagcgttatgtttaccct ctctttagcctactttagttcaggctattcaatcgaaacgtgcgaatataatatgtttgagtttaataatcgattagatcaacccttagaagaaa aagaagtaatcaaaattgttagaagtgcctattcagaaaactatcaaggggctaatagggaatacattaccattctttgcaaagcttgggta tcaagtgatttaaccagtaaagatttatttgtccgtcaagggtggtttaaattcaagaaaaaaagaagcgaacgtcaacgtgttcatttgtca gaatggaaagaagatttaatggcttatattagcgaaaaaagcgatgtatacaagccttatttagcgacgaccaaaaaagagattagagaa gtgctaggcattcctgaacggacattagataaattgctgaaggtactgaaggcgaatcaggaaattttctttaagattaaaccaggaagaa atggtggcattcaacttgctagtgttaaatcattgttgctatcgatcattaaattaaaaaaagaagaacgagaaagctatataaaggcgctg acagcttcgtttaatttagaacgtacatttattcaagaaactctaaacaaattggcagaacgccccaaaacggacccacaactcgatttgtt tagctacgatacaggctgaaaataaaacccgcactatgccattacatttatatctatgatacgtgtttgatttattgctggctagcttaattgc ttatatttacctgcaataaaggatttcttacttccattatactcccattttccaaaaacatacggggaacacgggaacttattgtacaggccac ctcatagttaatggtttcgagccttcctgcaatctcatccatggaaatatattcatccccctgccggcctattaatgtgacttttgtgcccggc ggatattcctgatccagctccaccataaattggtccatgcaaattcggccggcaattttcaggcgttttcccttcacaaggatgtcggtccc tttcaattttcggagccagccgtccgcatagcctacaggcaccgtcccgatccatgtgtctttttccgctgtgtactcggctccgtagctga cgctctcgccttttctgatcagtttgacatgtgacagtgtcgaatgcagggtaaatgccggacgcagctgaaacggtatctcgtccgac at gtcagcagacgggcgaaggccatacatgccgatgccgaatctgactgcattaaaaaagccttattcagccggagtccagcggcgctg ttcgcgcagtggaccattagattctttaacggcagcggagcaatcagctctttaaagcgctcaaactgcattaagaaatagcctctttattt tcatccgctgtcgcaaaatgggtaaatacccctttgcactttaaacgagggttgcggtcaagaattgccatcacgttctgaacttcttcctct gtttttacaccaagtctgttcatccccgtatcgaccttcagatgaaaatgaagagaaccttttttcgtgtggcgggctgcctcctgaagccat tcaacagaataacctgttaaggtcacgtcatactcagcagcgattgccacatactccgggggaaccgcgccaagcaccaatataggcg ccttcaatccctttttgcgcagtgaaatcgcttcatccaaaatggccacggccaagcatgaagcacctgcgtcaagagcagcctttgctgt ttctgcatcaccatgcccgtaggcgtttgctttcacaactgccatcaagtggacatgttcaccgatatgattttcatattgctgacanttcatt atcgcggacaagtcaatttccgcccacgtatctctgtaaaaaggttttgtgctcatggaaaactcctctcttttttcagaaaatcccagtacgt aattaagtatttgagaattaattttatattgattaatactaagtttacccagttttcacctaaaaaacaaatgatgagataatagctccaaaggct aaagaggactataccaactatttgttaattaa (SEQ ID NO: 21). This plasmid was sequenced at Genewiz facility from the E. coli strain on Feb. 20, 2008.

### Example 1

### Construction of Attenuated Listeria Strain-LmddΔactA and Insertion of the Human klk3 Gene in Frame to the hly Gene in the Lmdd and Lmdda Strains

The strain Lm dal dat (Lmdd) was attenuated by the irreversible deletion of the virulence factor, ActA. An in-frame deletion of actA in the Lmdaldat (Lmdd) background was constructed to avoid any polar effects on the expression of downstream genes. The Lm dal datΔactA contains the first 19 amino acids at the N-terminal and 28 amino acid residues of the C-terminal with a deletion of 591 amino acids of ActA.

The actA deletion mutant was produced by amplifying the chromosomal region corresponding to the upstream (657 bp-oligo's Adv 271/272) and downstream (625 bp-oligo's Adv 273/274) portions of actA and joining by PCR. The sequence of the primers used for this amplification is given in the Table 2. The upstream and downstream DNA regions of actA were cloned in the pNEB193 at the EcoRI/PstI restriction site and from this plasmid, the EcoRI/PstI was further cloned in the temperature sensitive plasmid pKSV7, resulting in AactA/pKSV7 (pAdv120).

TABLE 2
Sequence of primers that was used for the
amplification of DNA sequences upstream and
downstream of actA

| | | SEQ |
|---|---|---|
| | | ID |
| Primer | Sequence | NO: |
| Adv271- | cg GAATTCGGATCCgcgccaaatcattggttgattg | 22 |
| actAF1 | | |
| Adv272- | gcgaGTCGACgtcggggttaatcgtaatgcaattggc | 23 |
| actAR1 | | |
| Adv273- | gcgaGTCGACccatacgacgttaattcttgcaatg | 24 |
| actAF2 | | |
| Adv274- | gataCTGCAGGGATCCttcccttctcggtaatcagtcac | 25 |
| actAR2 | | |

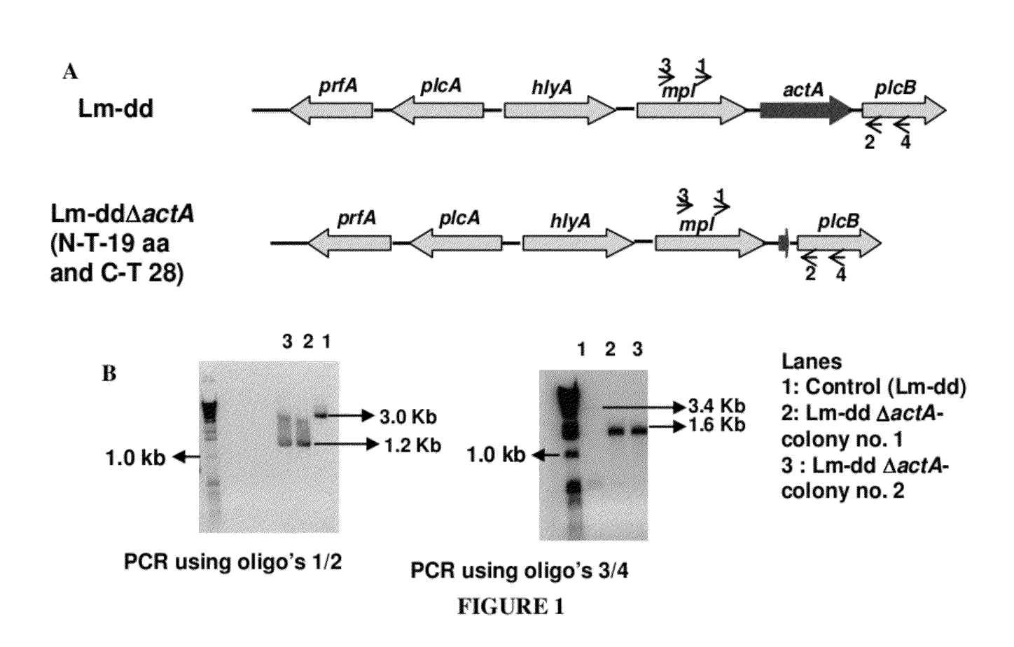

The deletion of the gene from its chromosomal location was verified using primers that bind externally to the actA deletion region, which are shown in FIG. 1 as primer 3 (Adv 305-tgggatggccaagaaattc, SEQ ID NO: 34) and primer 4 (Adv304-ctaccatgtcttccgttgcttg; SEQ ID NO: 35). The PCR analysis was performed on the chromosomal DNA isolated from Lmdd and LmddΔactA. The sizes of the DNA fragments after amplification with two different sets of primer pairs ½ and ¾ in Lmdd chromosomal DNA was expected to be 3.0 Kb and 3.4 Kb. On the other hand, the expected sizes of PCR using the primer pairs ½ and ¾ for the LmddΔactA was 1.2 Kb and 1.6 Kb. Thus, PCR analysis in FIG. 1 confirms that the 1.8 kb region of actA was deleted in the LmddΔactA strain. DNA sequencing was also performed on PCR products to confirm the deletion of actA containing region in the strain, LmddΔactA.

### Example 2

### Construction of the Antibiotic-Independent Episomal Expression System for Antigen Delivery by Lm Vectors

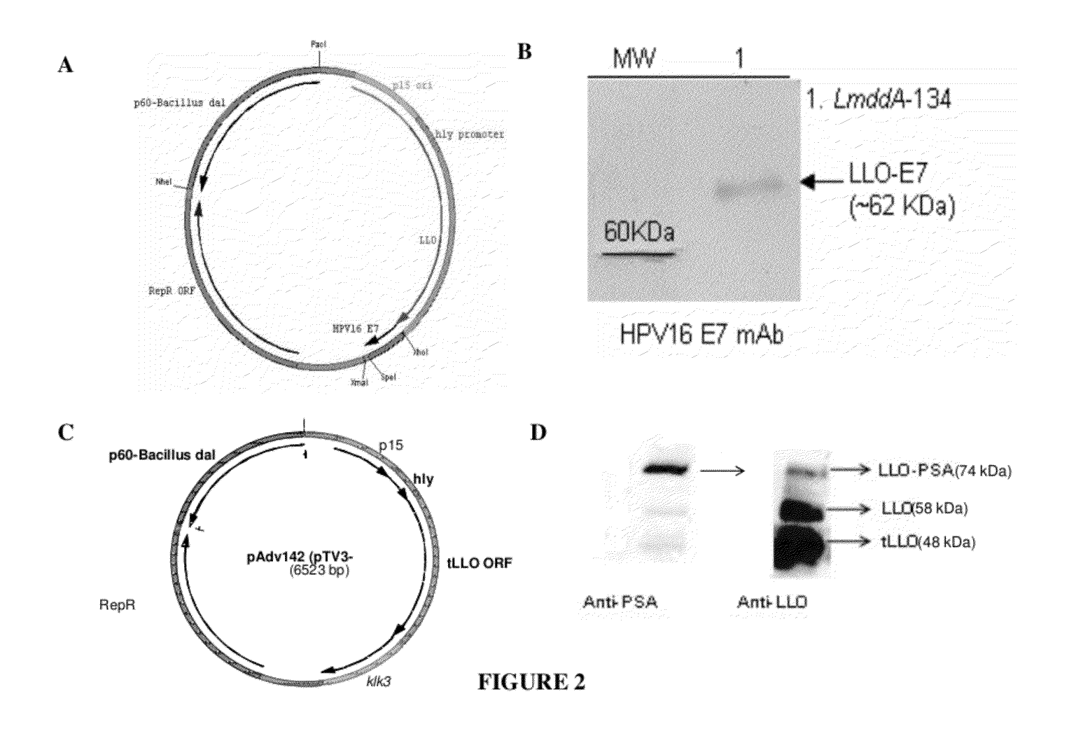

The antibiotic-independent episomal expression system for antigen delivery by Lm vectors (pAdv142) is the next generation of the antibiotic-free plasmid pTV3 (Verch et al., Infect Immun, 2004. 72 (11):6418-25, incorporated herein by reference). The gene for virulence gene transcription activator, prfA was deleted from pTV3 since Listeria strain Lmdd contains a copy of prfA gene in the chromosome. Additionally, the cassette for p60-Listeria dal at the NheI/PacI restriction site was replaced by p60-Bacillus subtilis dal resulting in plasmid pAdv134 (FIG. 2A). The similarity of the Listeria and Bacillus dal genes is ˜30%, virtually eliminating the chance of recombination between the plasmid and the remaining fragment of the dal gene in the Lmdd chromosome. The plasmid pAdv134 contained the antigen expression cassette tLLO-E7. The LmddA strain was transformed with the pADV134 plasmid and expression of the LLO-E7 protein from selected clones confirmed by Western blot (FIG. 2B). The Lmdd system derived from the 10403S wild-type strain lacks antibiotic resistance markers, except for the Lmdd streptomycin resistance.

Further, pAdv134 was restricted with XhoI/XmaI to clone human PSA, klk3 resulting in the plasmid, pAdv142. The new plasmid, pAdv142 (FIG. 2C, Table 1) contains Bacillus dal (B-Dal) under the control of Listeria p60 promoter. The shuttle plasmid, pAdv142 complemented the growth of both E. coli ala drx MB2159 as well as Listeria monocytogenes strain Lmdd in the absence of exogenous D-alanine. The antigen expression cassette in the plasmid pAdv142 consists of hly promoter and LLO-PSA fusion protein (FIG. 2C).

The plasmid pAdv142 was transformed to the Listeria background strains, Lmdd actA strain resulting in Lm-ddA-LLO-PSA. The expression and secretion of LLO-PSA fusion protein by the strain, Lm-ddA-LLO-PSA was confirmed by Western Blot using anti-LLO and anti-PSA antibody (FIG. 2D). There was stable expression and secretion of LLO-PSA fusion protein by the strain, Lm-ddA-LLO-PSA after two in vivo passages.

### Example 3

### In Vitro and In Vivo Stability of the Strain LmddA-LLO-PSA

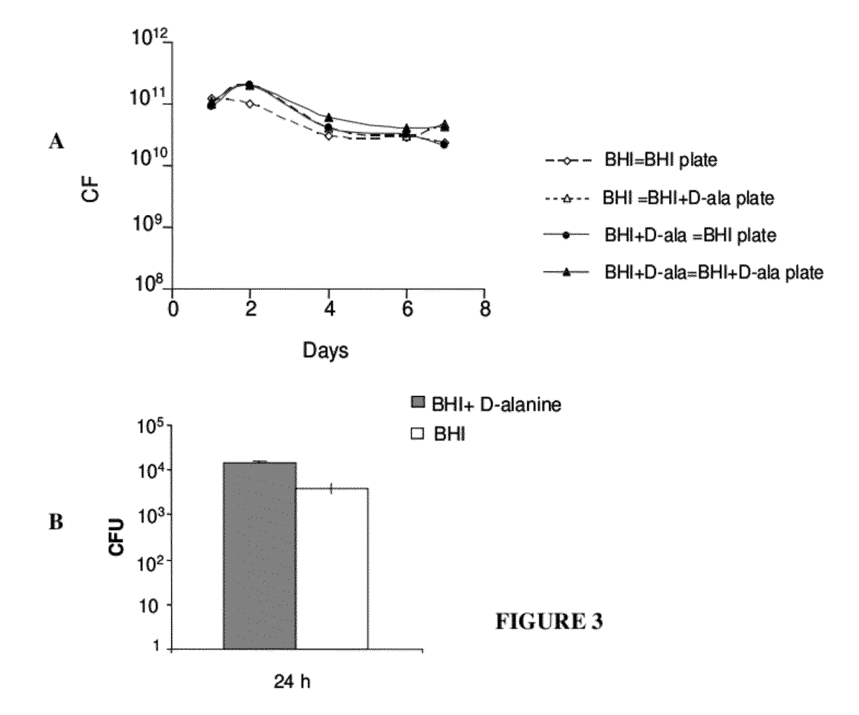

The in vitro stability of the plasmid was examined by culturing the LmddA-LLO-PSA Listeria strain in the presence or absence of selective pressure for eight days. The selective pressure for the strain LmddA-LLO-PSA is D-alanine. Therefore, the strain LmddA-LLO-PSA was passaged in Brain-Heart Infusion (BHI) and BHI+100 μg/ml D-alanine. CFUs were determined for each day after plating on selective (BHI) and non-selective (BHI+D-alanine) medium. It was expected that a loss of plasmid will result in higher CFU after plating on non-selective medium (BHI+D-alanine). As depicted in FIG. 3A, there was no difference between the number of CFU in selective and non-selective medium. This suggests that the plasmid pAdv142 was stable for at least 50 generations, when the experiment was terminated.

Plasmid maintenance in vivo was determined by intravenous injection of 5×10⁷ CFU LmddA-LLO-PSA, in C57BL/6 mice. Viable bacteria were isolated from spleens homogenized in PBS at 24 h and 48 h. CFUs for each sample were determined at each time point on BHI plates and BHI+100 μg/ml D-alanine. After plating the splenocytes on selective and non-selective medium, the colonies were recovered after 24 h. Since this strain is highly attenuated, the bacterial load is cleared in vivo in 24 h. No significant differences of CFUs were detected on selective and non-selective plates, indicating the stable presence of the recombinant plasmid in all isolated bacteria (FIG. 3B).

### Example 4

### In Vivo Passaging, Virulence and Clearance of the Strain LmddA-142

### (LmddA-LLO-PSA)

LmddA-142 is a recombinant Listeria strain that secretes the episomally expressed tLLO-PSA fusion protein. To determine a safe dose, mice were immunized with LmddA-LLO-PSA at various doses and toxic effects were determined LmddA-LLO-PSA caused minimum toxic effects (data not shown). The results suggested that a dose of 10⁸CFU of LmddA-LLO-PSA was well tolerated by mice. Virulence studies indicate that the strain LmddA-LLO-PSA was highly attenuated.

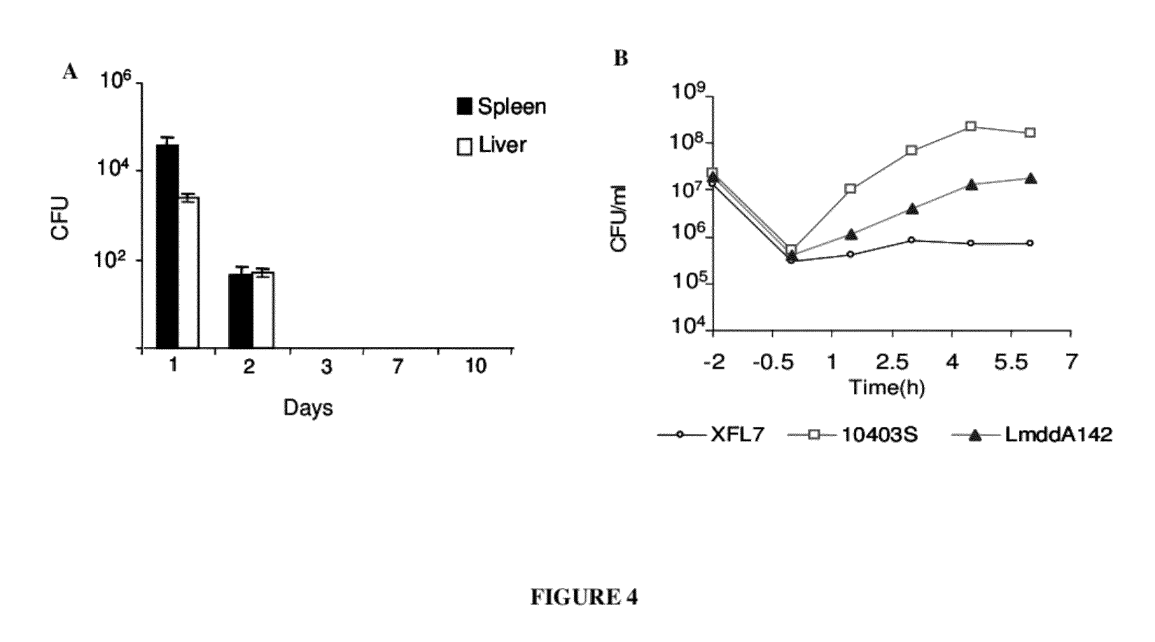

The in vivo clearance of LmddA-LLO-PSA after administration of the safe dose, 10⁸ CFU intraperitoneally in C57BL/6 mice, was determined. There were no detectable colonies in the liver and spleen of mice immunized with LmddA-LLO-PSA after day 2. Since this strain is highly attenuated, it was completely cleared in vivo at 48 h (FIG. 4A).

To determine if the attenuation of LmddA-LLO-PSA attenuated the ability of the strain LmddA-LLO-PSA to infect macrophages and grow intracellularly, we performed a cell infection assay. Mouse macrophage-like cell line such as J774A.1 were infected in vitro with Listeria constructs and intracellular growth was quantified. The positive control strain, wild type Listeria strain 10403S grows intracellularly, and the negative control XFL7, a prfA mutant, cannot escape the phagolysosome and thus does not grow in J774 cells. The intracytoplasmic growth of LmddA-LLO-PSA was slower than 10403S due to the loss of the ability of this strain to spread from cell to cell (FIG. 4B). The results indicate that LmddA-LLO-PSA has the ability to infect macrophages and grow intracytoplasmically.

### Example 5

### Immunogenicity of the Strain-LmddA-LLO-PSA in C57BL/6 Mice

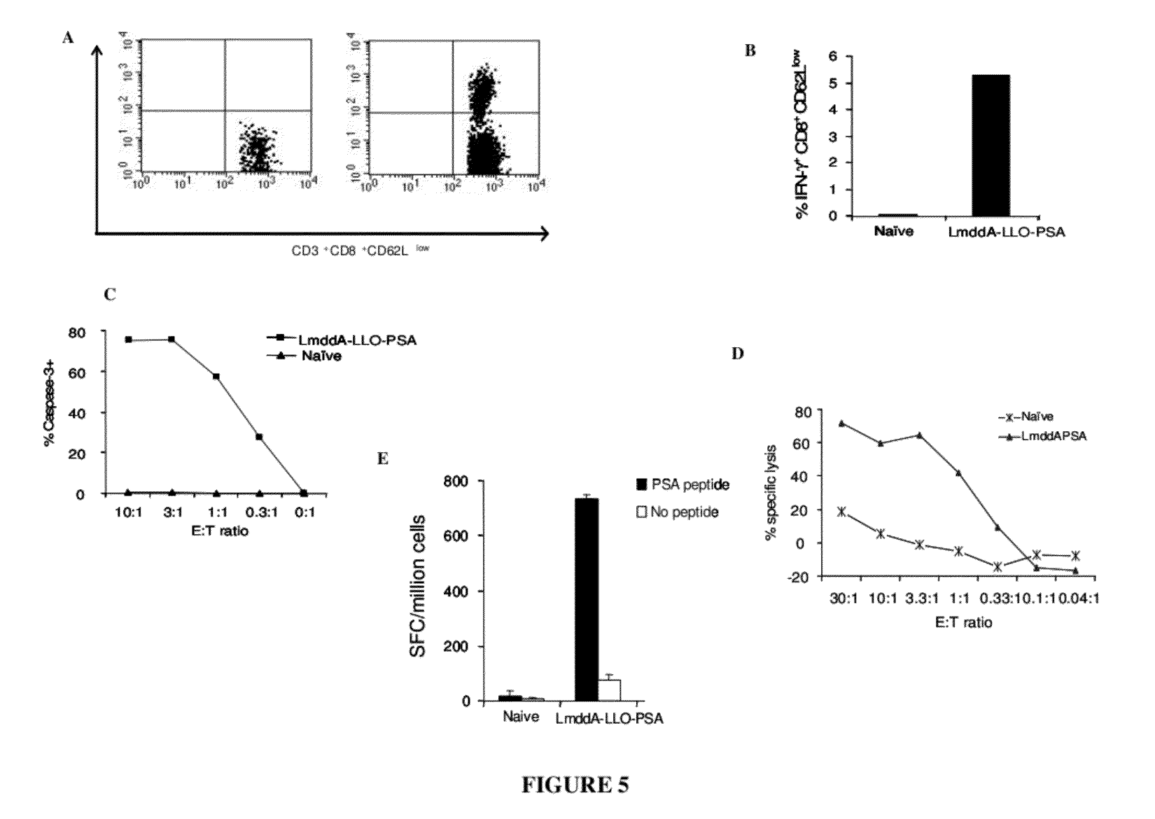

The PSA-specific immune responses elicited by the construct LmddA-LLO-PSA in C57BL/6 mice were determined using PSA tetramer staining. Mice were immunized twice with LmddA-LLO-PSA at one week intervals and the splenocytes were stained for PSA tetramer on day 6 after the boost. Staining of splenocytes with the PSA-specific tetramer showed that LmddA-LLO-PSA elicited 23% of PSA tetramer⁺CD8⁺CD62L^{low} cells (FIG. 5A).

The functional ability of the PSA-specific T cells to secrete IFN-γ after stimulation with PSA peptide for 5 h was examined using intracellular cytokine staining. There was a 200-fold increase in the percentage of CD8⁺CD62L^{low}IFN-γ secreting cells stimulated with PSA peptide in the LmddA-LLO-PSA group compared to the naïve mice (FIG. 5B), indicating that the LmddA-LLO-PSA strain is very immunogenic and primes high levels of functionally active PSA CD8⁺ T cell responses against PSA in the spleen.

To determine the functional activity of cytotoxic T cells generated against PSA after immunizing mice with LmddA-LLO-PSA, we tested the ability of PSA-specific CTLs to lyse cells EL4 cells pulsed with H-2D^{b} peptide in an in vitro assay. A FACS-based caspase assay (FIG. 5C) and Europium release (FIG. 5D) were used to measure cell lysis. Splenocytes of mice immunized with LmddA-LLO-PSA contained CTLs with high cytolytic activity for the cells that display PSA peptide as a target antigen.

Elispot was performed to determine the functional ability of effector T cells to secrete IFN-γ after 24 h stimulation with antigen. Using ELISpot, we observed there was a 20-fold increase in the number of spots for IFN-γ in splenocytes from mice immunized with LmddA-LLO-PSA stimulated with specific peptide when compared to the splenocytes of the naïve mice (FIG. 5E).

### Example 6

### Immunization with the LmddA-142 Strains Induces Regression of a Tumor Expressing PSA and Infiltration of the Tumor by PSA-Specific CTLs

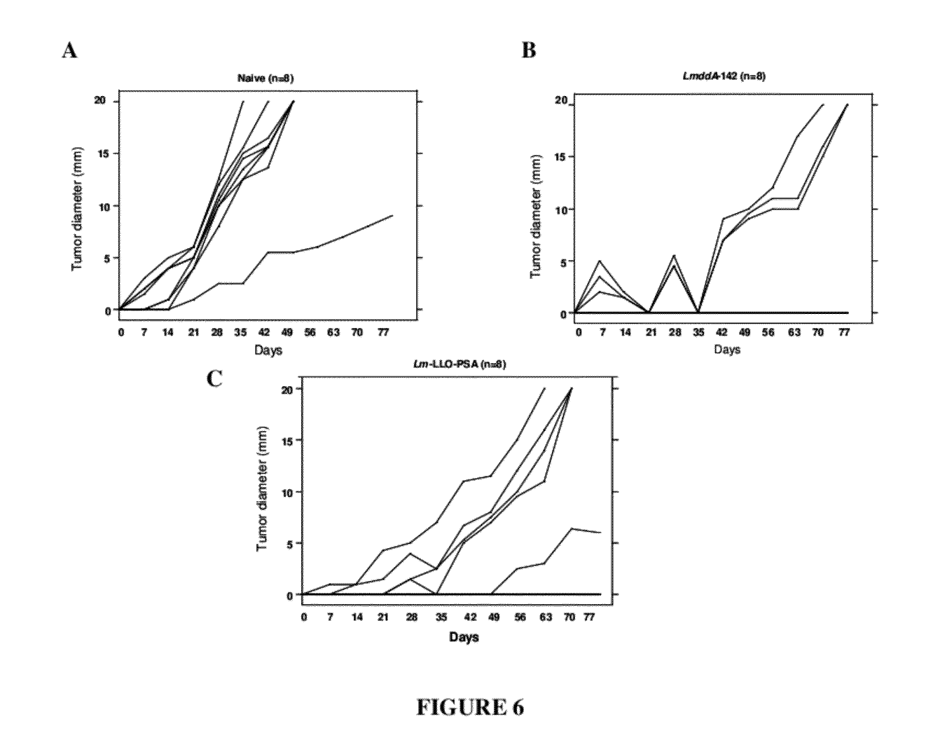

The therapeutic efficacy of the construct LmddA-142(LmddA-LLO-PSA) was determined using a prostrate adenocarcinoma cell line engineered to express PSA (Tramp-Cl-PSA (TPSA); Shahabi et al., 2008). Mice were subcutaneously implanted with 2×10⁶TPSA cells. When tumors reached the palpable size of 4-6 mm, on day 6 after tumor inoculation, mice were immunized three times at one week intervals with 10⁸ CFU LmddA-142, 10⁷ CFU Lm-LLO-PSA (positive control) or left untreated. The naïve mice developed tumors gradually (FIG. 6A). The mice immunized with LmddA-142 were all tumor-free until day 35 and gradually 3 out of 8 mice developed tumors, which grew at a much slower rate as compared to the naïve mice (FIG. 6B). Five out of eight mice remained tumor free through day 70. As expected, Lm-LLO-PSA-vaccinated mice had fewer tumors than naïve controls and tumors developed more slowly than in controls (FIG. 6C). Thus, the construct LmddA-LLO-PSA could regress 60% of the tumors established by TPSA cell line and slow the growth of tumors in other mice. Cured mice that remained tumor free were rechallenged with TPSA tumors on day 72.

Immunization of mice with the LmddA-142 can control the growth and induce regression of 7-day established Tramp-C1 tumors that were engineered to express PSA in more than 60% of the experimental animals (FIG. 6B), compared to none in the untreated group (FIG. 6A). The LmddA-142 was constructed using a highly attenuated vector (LmddA) and the plasmid pADV142 (Table 1).

Further, the ability of PSA-specific CD8 lymphocytes generated by the LmddA-LLO-PSA construct to infiltrate tumors was investigated. Mice were subcutaneously implanted with a mixture of tumors and matrigel followed by two immunizations at seven day intervals with naive or control (Lm-LLO-E7) Listeria, or with LmddA-LLO-PSA. Tumors were excised on day 21 and were analyzed for the population of CD8⁺CD62L^{low} PSA^{tetramer+} and CD4⁺CD25⁺FoxP3⁺ regulatory T cells infiltrating in the tumors.

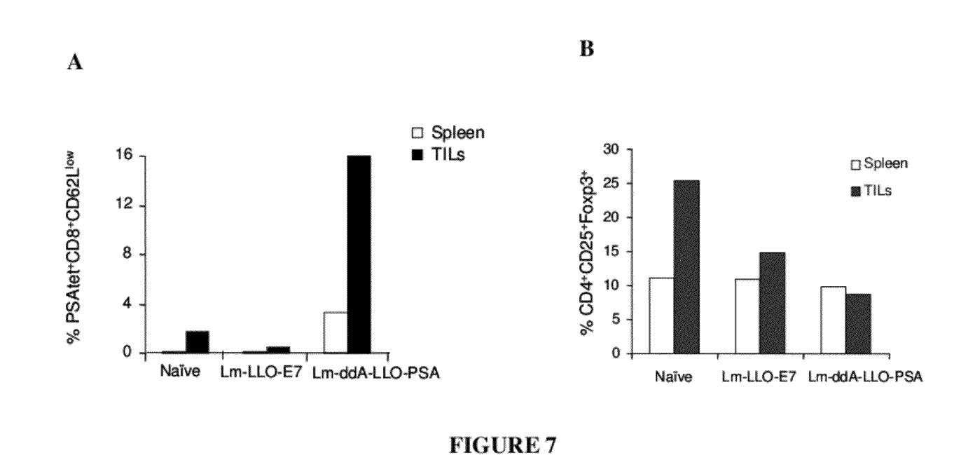

A very low number of CD8⁺CD62L^{low} PSA^{tetramer+} tumor infiltrating lymphocytes (TILs) specific for PSA that were present in the both naïve and Lm-LLO-E7 control immunized mice was observed. However, there was a 10-30-fold increase in the percentage of PSA-specific CD8⁺CD62L^{low} PSA^{tetramer+} TILs in the mice immunized with LmddA-LLO-PSA (FIG. 7A). Interestingly, the population of CD8⁺CD62L^{low} PSA^{tetramer+} cells in spleen was 7.5 fold less than in tumor (FIG. 7A).

In addition, the presence of CD4⁺/CD25⁺/Foxp3⁺ T regulatory cells(regs) in the tumors of untreated mice and Listeria immunized mice was determined Interestingly, immunization with Listeria resulted in a considerable decrease in the number of CD4⁺CD25⁺FoxP3⁺T-regs in tumor but not in spleen (FIG. 7B). However, the construct LmddA-LLO-PSA had a stronger impact in decreasing the frequency of CD4⁺CD25⁺FoxP3⁺ T-regs in tumors when compared to the naïve and Lm-LLO-E7 immunized group (FIG. 7B).

Thus, the LmddA-142 vaccine can induce PSA-specific CD8⁺ T cells that are able to infiltrate the tumor site (FIG. 7A). Interestingly, Immunization with LmddA-142 was associated with a decreased number of regulatory T cells in the tumor (FIG. 7B), probably creating a more favorable environment for an efficient anti-tumor CTL activity.

### Example 7

### Lmdd-143 and LmddA-143 Secretes a Functional LLO Despite the PSA Fusion

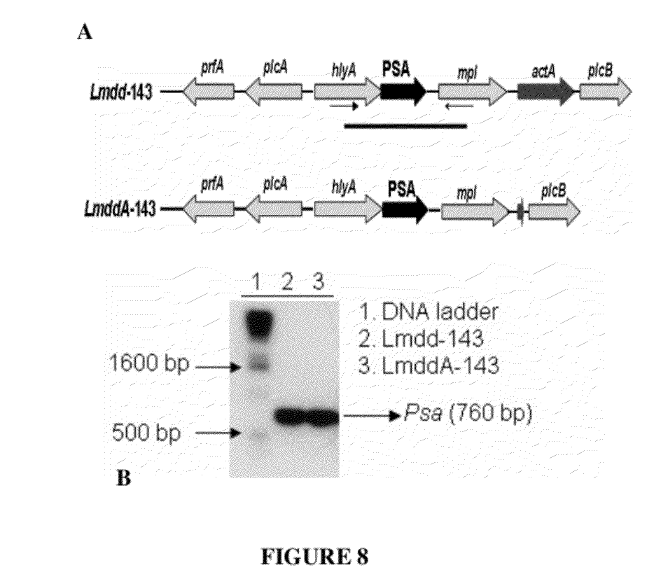

The Lmdd-143 and LmddA-143 contain the full-length human klk3 gene, which encodes the PSA protein, inserted by homologous recombination downstream and in frame with the hly gene in the chromosome. These constructs were made by homologous recombination using the pKSV7 plasmid (Smith and Youngman, Biochimie 1992; 74 (7-8) p705-711), which has a temperature-sensitive replicon, carrying the hly-klk3-mpl recombination cassette. Because of the plasmid excision after the second recombination event, the antibiotic resistance marker used for integration selection is lost. Additionally, the actA gene is deleted in the LmddA-143 strain (FIG. 8A). The insertion of klk3 in frame with hly into the chromosome was verified by PCR (FIG. 8B) and sequencing (data not shown) in both constructs.

One important aspect of these chromosomal constructs is that the production of LLO-PSA would not completely abolish the function of LLO, which is required for escape of Listeria from the phagosome, cytosol invasion and efficient immunity generated by L. monocytogenes. Western-blot analysis of secreted proteins from Lmdd-143 and LmddA-143 culture supernatants revealed an ˜81 kDa band corresponding to the LLO-PSA fusion protein and an ˜60 kDa band, which is the expected size of LLO (FIG. 9A), indicating that LLO is either cleaved from the LLO-PSA fusion or still produced as a single protein by L. monocytogenes, despite the fusion gene in the chromosome. The LLO secreted by Lmdd-143 and LmddA-143 retained 50% of the hemolytic activity, as compared to the wild-type L. monocytogenes 10403S (FIG. 9B). In agreement with these results, both Lmdd-143 and LmddA-143 were able to replicate intracellularly in the macrophage-like J774 cell line (FIG. 9C).

### Example 8

### Both Lmdd-143 and LmddA-143 Elicit Cell-Mediated Immune Responses Against the PSA Antigen

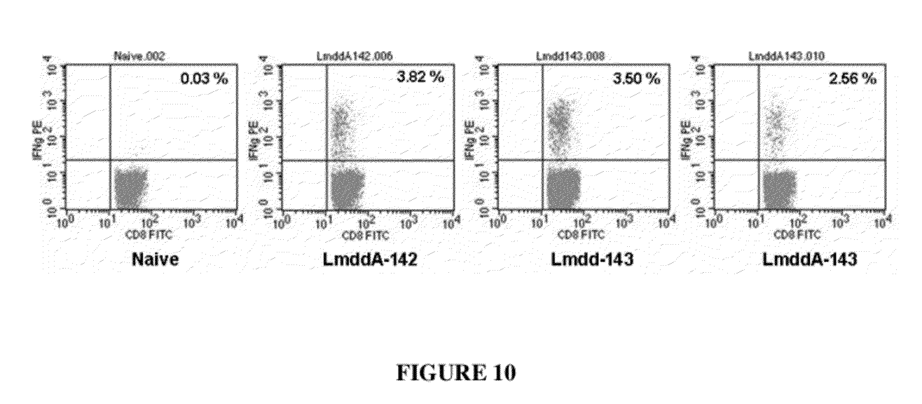

After showing that both Lmdd-143 and LmddA-143 are able to secrete PSA fused to LLO, we investigated if these strains could elicit PSA-specific immune responses in vivo. C57B1/6 mice were either left untreated or immunized twice with the Lmdd-143, LmddA-143 or LmddA-142. PSA-specific CD8⁺ T cell responses were measured by stimulating splenocytes with the PSA₆₅₋₇₄ peptide and intracellular staining for IFN-γ. As shown in FIG. 10, the immune response induced by the chromosomal and the plasmid-based vectors is similar.

### Example 9

### A recombinant Lm Strain Secreting a LLO-HMW-MAA Fusion Protein Results in a Broad Antitumor Response

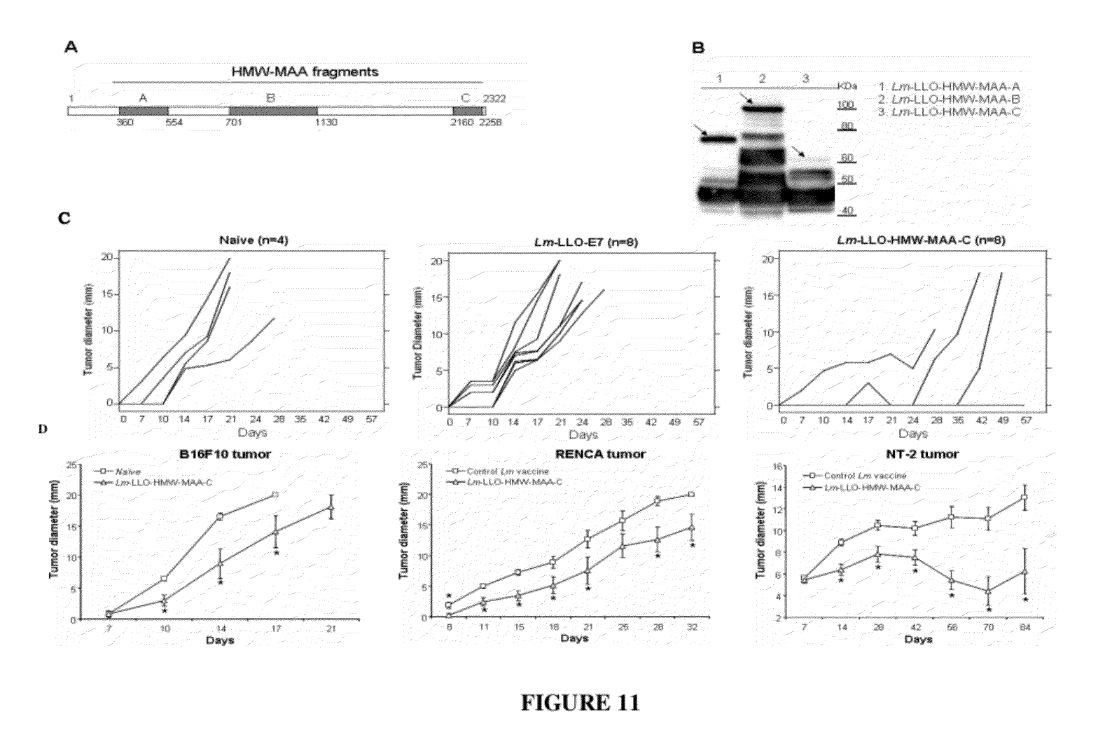

Three Lm-based vaccines expressing distinct HMW-MAA fragments based on the position of previously mapped and predicted HLA-A2 epitopes were designed (FIG. 11A). The Lm-tLLO-HMW-MMA₂₁₆₀₋₂₂₅₈ (also referred as Lm-LLO-HMW-MAA-C) is based on the avirulent Lm XFL-7 strain and a pGG55-based plasmid. This strain secretes a ˜62 kDa band corresponding to the tLLO-HMW-MAA₂₁₆₀₋₂₂₅₈ fusion protein (FIG. 11B). The secretion of tLLO-HMW-MAA₂₁₆₀₋₂₂₅₈ is relatively weak likely due to the high hydrophobicity of this fragment, which corresponds to the HMW-MAA transmembrane domain. Using B16F10 melanoma cells transfected with the full-length HMW-MAA gene, we observed that up to 62.5% of the mice immunized with the Lm-LLO-HMW-MAA-C could impede the growth of established tumors (FIG. 11C). This result shows that HMW-MAA can be used as a target antigen in vaccination strategies. Interestingly, we also observed that immunization of mice with Lm-LLO-HMW-MAA-C significantly impaired the growth of tumors not engineered to express HMW-MAA, such as B16F10, RENCA and NT-2 (FIG. 11D), which were derived from distinct mouse strains. In the NT-2 tumor model, which is a mammary carcinoma cell line expressing the rat HER-2/neu protein and is derived from the FVB/N transgenic mice, immunization with Lm-LLO-HMW-MAA-C 7 days after tumor inoculation not only impaired tumor growth but also induced regression of the tumor in 1 out of 5 mice (FIG. 11D).

### Example 10

### Immunization of Mice with Lm-LLO-HMW-MAA-C Induces Infiltration of the Tumor Stroma by CD8⁺ T Cells and a Significant Reduction in the Pericyte Coverage in the Tumor Vasculature

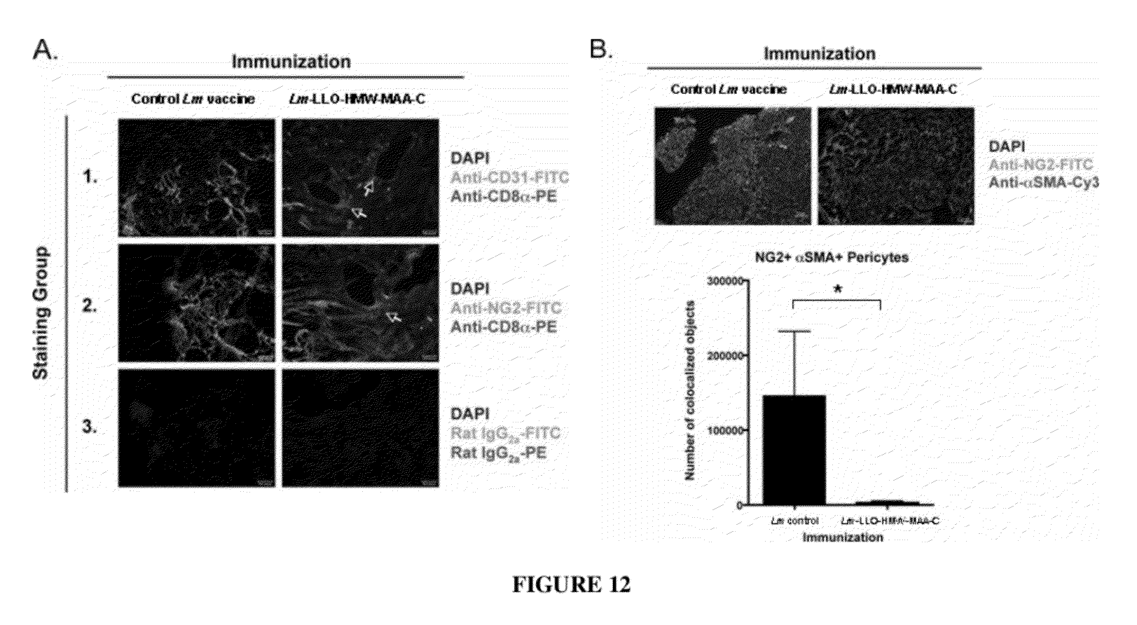

Although NT-2 cells do not express the HMW-MAA homolog NG2, immunization of FVB/N mice with Lm-LLO-HMW-MAA-C significantly impaired the growth of NT-2 tumors and eventually led to tumor regression (FIG. 11D). This tumor model was used to evaluate CD8⁺ T cells and pericytes in the tumor site by immunofluorescence. Staining of NT-2 tumor sections for CD8 showed infiltration of CD8⁺ T cells into the tumors and around blood vessels in mice immunized with the Lm-LLO-HMW-MAA-C vaccine, but not in mice immunized with the control vaccine (FIG. 2A). Pericytes in NT-2 tumors were also analyzed by double staining with αSMA and NG2 (murine homolog of HMW-MAA) antibodies. Data analysis from three independent NT-2 tumors showed a significant decrease in the number of pericytes in mice immunized with Lm-LLO-HMW-MAA-C, as compared to control (P≦0.05) (FIG. 12B). Similar results were obtained when the analysis was restricted to cells stained for αSMA, which is not targeted by the vaccine (data not shown). Thus, Lm-LLO-HMW-MAA-C vaccination impacts blood vessel formation in the tumor site by targeting pericytes.

### Example 11

### Development of a Recombinant L. monocytogenes Vector with Enhanced Anti-Tumor Activity by Concomitant Expression and Secretion of LLO-PSA and tLLO-HMW-MAA₂₁₆₀₋₂₂₅₈ Fusion Proteins, Eliciting Immune Responses to Both Heterologous Antigens

### Materials and Methods:

Construction of the pADV172 plasmid. The HMW-MAA-C fragment is excised from a pCR2.1-HMW-MAA₂₁₆₀₋₂₂₅₈ plasmid by double digestion with XhoI and XmaI restriction endonucleases. This fragment is cloned in the pADV134 plasmid already digested with XhoI and XmaI to excise the E7 gene. The pADV172 plasmid is electroporated into electrocompetent the dal⁽⁻⁾dat⁽⁻⁾ E. coli strain MB2159 and positive clones screened for RFLP and sequence analysis.

Construction of Lmdd-143/172, LmddA-143/172 and the control strains LmddA-172, Lmdd-143/134 and LmddA-143/134. Lmdd, Lmdd-143 and LmddA-143 is transformed with either pADV172 or pADV134 plasmid. Transformants are selected on Brain-Heart Infusion-agar plates supplemented with streptomycin (250 μg/ml) and without D-alanine (BHIs medium). Individual clones are screened for LLO-PSA, tLLO-HMW-MAA₂₁₆₀₋₂₂₅₈ and tLLO-E7 secretion in bacterial culture supernatants by Western-blot using an anti-LLO, anti-PSA or anti-E7 antibody. A selected clone from each strain will be evaluated for in vitro and in vivo virulence. Each strain is passaged twice in vivo to select the most stable recombinant clones. Briefly, a selected clone from each construct is grown and injected i.p. to a group of 4 mice at 1×10⁸ CFU/mouse. Spleens are harvested on days 1 and 3, homogenized and plated on BHIs-agar plates. After the first passage, one colony from each strain is selected and passaged in vivo for a second time. To prevent further attenuation of the vector, to a level impairing its viability, constructs in two vectors with distinct attenuation levels (Lmdd-143/172, LmddA-143/172) are generated.

In vitro virulence determination by intracellular replication in J774 cells. Uptake of Lm by macrophages, followed by cytosolic invasion and intracellular proliferation are required for successful antigen delivery and presentation by Lm-based vaccines. An in vitro invasion assay, using a macrophage-like J774 cell line is used to test these properties in new recombinant Lm strains. Briefly, J774 cells are infected for 1 hour in medium without antibiotics at MOI of 1:1 with either the control wild-type Lm strain 10403S or the new Lm strains to be tested. Extracellular bacteria are killed by 1 hour incubation in medium 10 μg/ml of gentamicin. Samples are harvested at regular intervals and cells lysed with water. Ten-fold serial dilutions of the lysates are plated in duplicates on BHIs plates and colony-forming units (CFU) counted in each sample.

In vivo virulence studies. Groups of four C57BL/6 mice (7 weeks old) are injected i.p. with two different doses (1×10⁸ and 1×10⁹ CFUs/dose) of Lmdd-143/172, LmddA-143/172, LmddA-172, Lmdd-143/134 or LmddA-143/134 strains. Mice are followed-up for 2 weeks for survival and LD₅₀ estimation. An LD₅₀ of >1×10⁸ constitutes an acceptable value based on previous experience with other Lm-based vaccines.

### Results

Once the pADV172 plasmid is successfully constructed, it is sequenced for the presence of the correct HMW-MAA sequence. This plasmid in these new strains express and secrete the LLO fusion proteins specific for each construct. These strains are highly attenuated, with an LD50 of at least 1×10⁸ CFU and likely higher than 1×10⁹ CFU for the actA-deficient (LmddA) strains, which lack the actA gene and consequently the ability of cell-to-cell spread. The construct is tested and the one that has a better balance between attenuation and therapeutic efficacy is selected.

### Example 12

### Detection of Immune Responses and Anti-Tumor Effects Elicited Upon Immunization with Lmdd-143/172 and LmddA-143/172

Immune responses to PSA and HMW-MAA are studied in mice upon immunization with Lmdd-143/172 and LmddA-143/172 strains using standard methods, such as detection of IFN-γ production and specific CTL activity against these antigens. The therapeutic efficacy of dual-expression vectors are tested in the TPSA23 tumor model.

Intracellular cytokine staining for IFN-γ. C57BL/6 mice (3 mice per treatment group) are immunized twice at 1-week intervals with the Lmdd-143/172 and LmddA-143/172 strains. As controls for this experiment, mice are immunized with Lmdd-143, LmddA-143, LmddA-142, LmddA-172, Lmdd-143/134, LmddA-143/134 or left untreated (naïve group). Spleens are harvested after 7 days and a single cell suspension of splenocytes are prepared. These splenocytes are plated at 2×10⁶ cells/well in a round bottom 96-well plate, in freshly prepared complete RPMI medium with IL-2 (50 U/ml) and stimulated with either the PSA H-2Db peptide, HCIRNKSVIL, (SEQ ID NO: 32), or the HPV16 E7 H-2Db control peptide RAHYNIVTF (SEQ ID NO: 33) at a final concentration of 1 μM. Since HMW-MAA-epitopes have not been mapped in the C57B1/6 mouse, HMW-MAA-specific immune responses are detected by incubating 2×10⁶ splenocytes with 2×10⁵ EL4-HMW-MAA cells. The cells are incubated for 5 hours in the presence of monensin to retain the intracellular IFN-γ in the cells. After incubation, cells are stained with anti-mouse CD8-FITC, CD3-PerCP, CD62L-APC antibodies. They are then permeabilized and stained for IFNγ-PE and analyzed in a four-color FACS Calibur (BD Biosciences).

Cytotoxicity assay. To investigate the effector activity of the PSA and HMW-MAA specific T cells generated upon vaccinations, isolated splenocytes are incubated for 5 days in complete RPMI medium containing 20 U/ml of mouse IL-2 (Sigma), in the presence of stimulator cells (mitomycin C treated MC57G cells infected with either PSA or HMW-MAA vaccinia). For the cytotoxicity assay, EL4 target cells are labeled for 15 minutes with DDAO-SE (0.6 μM) (Molecular Probes) and washed twice with complete medium. The labeled target cells are pulsed for 1 hour with either the PSA H-2Db peptide, or the HPV16 E7 H-2Db control peptide, at a final concentration of 5 μM. For HMW-MAA-specific cytotoxic responses, the EL4-HMW-MAA cells are used as targets. The cytotoxicity assay is performed for 2 hours by incubating the target cells (T) with effector cells (E) at different E:T ratios for 2-3 hours. Cells are fixed with formalin, permeabilized and stained for cleaved caspase-3 to detect induction of apoptosis in the target cells.

Anti-tumor efficacy. The anti-tumor efficacy of the Lmdd-143/172 and LmddA-143/172 strains are compared to that of LmddA-142 and LmddA-172, using the T-PSA23 tumor model (TrampC-1/PSA). Groups of 8 male C57BL/6 mice (6-8 weeks old) are inoculated s.c. with 2×10⁶ T-PSA23 cells and 7 days later immunized i.p. with 0.1×LD50 dose of Lmdd-143/172, LmddA-143/172, LmddA-142 and LmddA-172. As controls, mice are either left untreated or immunized with an Lm control strain (LmddA-134). Each group receives two additional doses of the vaccines with 7 day intervals. Tumors are monitored for 60 days or until they reach a size of 2 cm, at which point mice are sacrificed.

### Results

Immunization of mice with LmddA-172 results in the induction of specific responses against HMW-MAA. Similarly, Lmdd-143/172 and LmddA-143/172 elicits an immune response against PSA and HMW-MAA that is comparable to the immune responses generated by L. monocytogenes vectors expressing each antigen individually Immunization of T-PSA-23-bearing mice with the Lmdd-143/172 and LmddA-143/172 results in a better anti-tumor therapeutic efficacy than the immunization with either LmddA-142 or LmddA-172.

### Example 13

### Immunization with Either Lmdd-143/172 or LmddA-143/172 Results in Pericyte Destruction, Up-Regulation of Adhesion Molecules in Endothelial Cells and Enhanced Infiltration of TILs Specific for PSA

Characterization of tumor infiltrating lymphocytes and endothelial cell-adhesion molecules induced upon immunization with Lmdd-143/172 or LmddA-143/172. The tumors from mice immunized with either Lmdd-143/172 or LmddA-143/172 are analyzed by immunofluorescence to study expression of adhesion molecules by endothelial cells, blood vessel density and pericyte coverage in the tumor vasculature, as well as infiltration of the tumor by immune cells, including CD8 and CD4 T cells. TILs specific for the PSA antigen are characterized by tetramer analysis and functional tests.

Analysis of tumor infiltrating lymphocytes (TILs). TPSA23 cells embedded in matrigel are inoculated s.c in mice (n=3 per group), which are immunized on days 7 and 14 with either Lmdd-143/172 or LmddA-143/172, depending on which one is the more effective according to results obtained in anti-tumor studies. For comparison, mice are immunized with LmddA-142, LmddA-172, a control Lm vaccine or left untreated. On day 21, the tumors are surgically excised, washed in ice-cold PBS and minced with a scalpel. The tumors are treated with dispase to solubilize the Matrigel and release single cells for analysis. PSA-specific CD8⁺ T cells are stained with a PSA65-74 H-2Db tetramer-PE and anti-mouse CD8-FITC, CD3-PerCP-Cy5.5 and CD62L-APC antibodies. To analyze regulatory T cell in the tumor, TILs are stained with CD4-FITC, CD3-PerCP-Cy5.5 and CD25-APC and subsequently permeabilized for FoxP3 staining (anti-FoxP3-PE, Milteny Biotec). Cells are analyzed by a FACS Calibur cytometer and CellQuestPro software (BD Biosciences).

Immunofluorescence. On day 21 post tumor inoculation, the TPSA23 tumors embedded in matrigel are surgically excised and a fragment immediately cryopreserved in OCT freezing medium. The tumor fragments are cryosectioned for 8-10 μm thick sections. For immunofluorescence, samples are thawed and fixed using 4% formalin. After blocking, sections are stained with antibodies in blocking solution in a humidified chamber at 37° C. for 1 hour. DAPI (Invitrogen) staining are performed according to manufacturer instructions. For intracellular stains (αSMA), incubation is performed in PBS/0.1% Tween/1% BSA solution. Slides are cover-slipped using a mounting solution (Biomeda) with anti-fading agents, set for 24 hours and kept at 4° C. until imaging using Spot Image Software (2006) and BX51 series Olympus fluorescent microscope. CD8, CD4, FoxP3, αSMA, NG2, CD31, ICAM-1, VCAM-1 and VAP-1 are evaluated by immunofluorescence.

Statistical analysis: Non-parametric Mann-Whitney and Kruskal-Wallis tests are applied to compare tumor sizes among different treatment groups. Tumor sizes are compared at the latest time-point with the highest number of mice in each group (8 mice). A p-value of less than 0.05 is considered statistically significant in these analyses.

### Results

Immunization of TPSA23-bearing mice with the Lmdd-143/172 and LmddA-143/172 results in higher numbers of effector TILs specific to PSA and also decreases pericyte coverage of the tumor vasculature. Further, cell-adhesion markers are significantly up-regulated in immunized mice.

### Example 14

### Construction of Dual Plasmid that Concomitantly Delivers Two Antigens

DNA corresponding to the actA promoter region and 1-233 amino acids of N-terminus of ActA will be amplified from Listeria genomic DNA by Polymerase Chain Reaction (PCR) using the following primers ActA-F-5′-atcccgggtgaagcttgggaagcagttggg-3′(XmaI) (SEQ ID NO: 6) and ActA-R— attctagatttatcacgtacccatttccccgc(XbaI) (SEQ ID NO:27). The restriction sites used for cloning are underlined. XmaI/XbaI segment will be cloned in plasmid pNEB193 to create pNEB193-ActA. Further antigen 2, which is Chimera Her2 will be PCR amplified using the primers Ch-Her2-F-5′-attctagaacccacctggacatgctccgccac-3′(XbaI) (SEQ ID NO: 28) and Ch-Her2-R-5′-gtcgacactagtctagtggtgatggtgatgatggagctcagatctgtctaagaggcagccatagggc-3′ (RE sites-SalI-SpeI-SacI-BglII) (SEQ ID NO: 29). The XbaI and SalI fragment of Ch-Her2 will be cloned in the plasmid pNEB193-ActA to create pNEB193-ActA-Ch-Her2 plasmid. His tag DNA sequence is included in the Ch-Her2 reverse primer sequence between Sad and SpeI restriction site. The XmaI/SpeI fragment corresponding to tActA-Ch-Her2-His from the plasmid pNEB193-ActA-Ch-Her2 will be excised for cloning in XmaI/SpeI restricted pAdv134 to create dual plasmid.

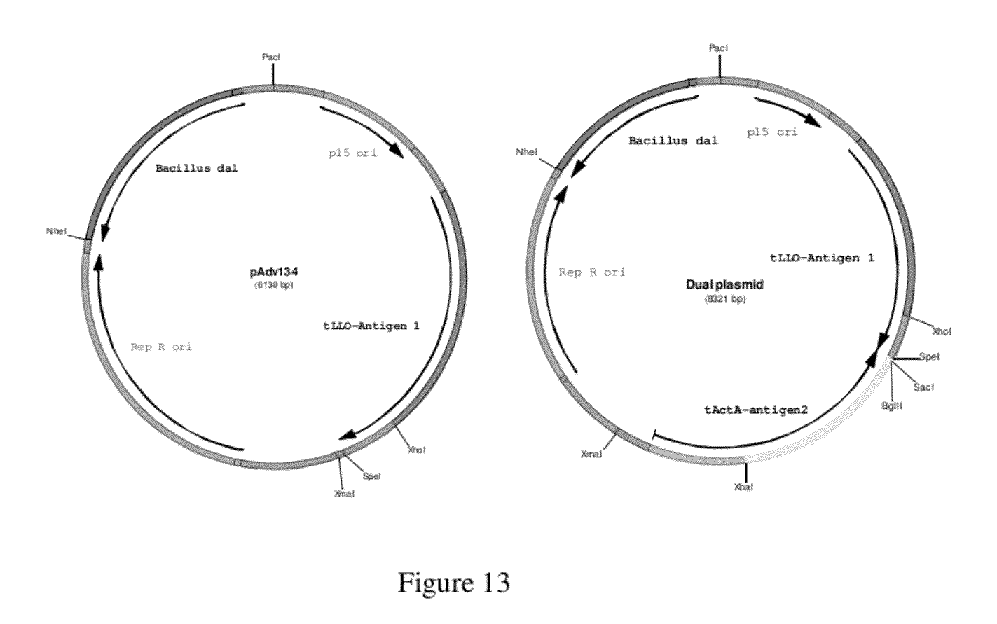

A Listeria based plasmid that delivers two recombinant antigens concomitantly as fusion proteins is generated. The two fusion proteins that are expressed by this plasmid include tLLO-antigen 1 and tActA-antigen 2. The expression and secretion of the antigen 1 is under the control of hly promoter and LLO signal sequence and it is expressed as a fusion to non-hemolytic fragment of Listeriolysin O (truncated LLO or tLLO). The expression and secretion of antigen 2 is under the control of actA promoter and ActA signal sequence and it is expressed as fusion to 1-233 amino acids of ActA (truncated ActA or tActA). The construction of antibiotic—marker free plasmid pAdv134 has been described previously and it contains the gene cassette for the expression of tLLO-antigen 1 fusion protein. The SpeI and Xma I restriction sites present downstream of the tLLO-antigen 1 in pAdv134 are used for the cloning of actA promoter-tActA-antigen 2 DNA segment (FIG. 13). The restriction sites XbaI, Sad and BglII are added in the cassette to facilitate cloning of the antigen 2 insert at XbaI/SacI or XbaI/BglII. A DNA sequence coding for His tag is added after Sad site to facilitate the detection of tActA-antigen 2-his fusion protein. The dual plasmid is able to concomitantly express and secrete two different antigens as fusion proteins.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A recombinant nucleic acid sequence comprising a first and at least a second open reading frame each encoding a first and at least a second polypeptide, wherein said first and said second polypeptide each comprise a heterologous antigen or a functional fragment thereof fused to an N-terminal truncated LLO polypeptide, an N-terminal ActA polypeptide, or PEST-peptide, or a functional fragment thereof.

2. The recombinant nucleic acid sequence of claim 1, wherein said nucleic acid further comprises a third open reading frame encoding a third polypeptide, wherein said third polypeptide comprises a heterologous antigen or a functional fragment thereof fused to an endogenous PEST-containing polypeptide.

3. (canceled)

4. The recombinant nucleic acid sequence of claim 1, wherein said first, or said at least second heterologous antigen or functional fragment thereof is expressed by a tumor cell.

5. The recombinant nucleic acid sequence of claim 2, wherein said third heterologous antigen or functional fragment thereof is expressed by a tumor cell.

6. The recombinant nucleic acid sequence of claim 1, wherein said first, or said at least second polypeptide comprises an angiogenic antigen or an antigen associated with tumor evasion or resistance to cancer or an antigen associated with the local tissue environment that is further associated with the development or metastasis of cancer.

7. (canceled)

8. (canceled)

9. A vaccine comprising a recombinant Listeria strain further comprising the recombinant nucleic acid of claim 1 and an adjuvant, cytokine, chemokine, or a combination thereof.

10. A nucleic acid library comprising the recombinant nucleic acid sequence of claim 1.

11. A recombinant Listeria strain comprising an episomal recombinant nucleic acid molecule, said nucleic acid molecule comprising a first and at least a second open reading frame each encoding a first and at least a second polypeptide, wherein said first and said at least second polypeptide each comprise a heterologous antigen or a functional fragment thereof fused to an N-terminal ActA polypeptide, or PEST-peptide, or a functional fragment thereof.

12. The recombinant Listeria strain of claim 11, wherein said nucleic acid further comprises a third open reading frame encoding a third polypeptide, wherein said third polypeptide comprises a heterologous antigen or a functional fragment thereof fused to an N-terminal truncated LLO polypeptide, N-terminal ActA polypeptide, or PEST-peptide, or a functional fragment thereof.

13. (canceled)

14. (canceled)

15. The recombinant Listeria strain of claim 11, wherein said first, or said at least second heterologous antigen or functional fragment thereof is expressed by a tumor cell.

16. The recombinant Listeria strain of claim 12, wherein said third heterologous antigen or functional fragment thereof is expressed by a tumor cell.

17. The recombinant Listeria strain of claim 11, wherein said first, or said at least second polypeptide comprises an angiogenic antigen or an antigen associated with tumor evasion or resistance to cancer or an antigen associated with the local tissue environment that is further associated with the development or metastasis of cancer.

18. (canceled)

19. (canceled)

20. (canceled)

21. The recombinant Listeria strain of claim 12, wherein said recombinant Listeria strain is an auxotrophic Listeria strain comprising a metabolic enzyme that complements the auxotrophy of said auxotrophic Listeria strain.

22. The recombinant Listeria strain of claim 21, wherein said auxotrophic Listeria strain is a dal/dat mutant.

23. (canceled)

24. The recombinant Listeria strain of claim 23, wherein said metabolic enzyme is an amino acid metabolism enzyme.

25. (canceled)

26. The recombinant Listeria strain of claim 23, wherein said metabolic enzyme is an alanine racemase enzyme.

27. The recombinant Listeria strain of claim 23, wherein said metabolic enzyme is a D-amino acid transferase enzyme.

28. The recombinant Listeria strain of claim 11, wherein said recombinant Listeria strain has been passaged through an animal host.

29. The recombinant Listeria strain of claim 11, wherein said recombinant Listeria strain is a recombinant Listeria monocytogenes strain.

30. A vaccine comprising the recombinant Listeria strain of claim 11 and an adjuvant, cytokine, chemokine, or a combination thereof.

31. A recombinant Listeria strain comprising a first integrated recombinant nucleic acid molecule comprising a first open reading frame encoding a polypeptide, wherein said polypeptide comprises a heterologous antigenic or a functional fragment thereof fused to an N-terminal truncated LLO polypeptide, an N-terminal ActA polypeptide, or PEST-peptide, or a functional fragment thereof, wherein said first nucleic acid molecule is integrated into said Listeria genome, wherein said Listeria strain further comprises an episomal recombinant nucleic acid molecule comprising a first and at least a second open reading frame each encoding a first and at least a second polypeptide, and wherein said first and said at least second polypeptide each comprise a heterologous antigen or a functional fragment thereof fused to said N-terminal truncated LLO polypeptide, an N-terminal ActA polypeptide, or PEST-peptide, or a functional fragment thereof.

32. The recombinant Listeria strain of claim 31, wherein said episomal bivalent recombinant nucleic acid further comprises a third open reading frame encoding a third polypeptide, wherein said third polypeptide comprises a heterologous antigen or a functional fragment thereof fused to an endogenous PEST-containing polypeptide.

33. (canceled)

34. (canceled)

35. The recombinant Listeria strain of claim 31, wherein said first, or said at least second heterologous antigen is expressed by a tumor cell.

36. The recombinant Listeria strain of claim 31, wherein said first, or said at least second polypeptide comprises an angiogenic antigen or an antigen associated with tumor evasion or resistance to cancer or an antigen is associated with the local tissue environment that is further associated with the development or metastasis of cancer.

37. (canceled)

38. (canceled)

39. The recombinant Listeria strain of claim 31, wherein said first nucleic acid molecule is a vector designed for site-specific homologous recombination into the Listeria genome.

40. (canceled)

41. The recombinant Listeria strain of claim 32, wherein said recombinant Listeria strain is an auxotrophic Listeria strain, comprising an episomal expression vector comprising a metabolic enzyme that complements the auxotrophy of said auxotrophic Listeria strain.

42. The recombinant Listeria strain of claim 41, wherein said auxotrophic Listeria strain is a dal/dat mutant.

43. (canceled)

44. The recombinant Listeria strain of claim 43, wherein said metabolic enzyme is an amino acid metabolism enzyme.

45. (canceled)

46. The recombinant Listeria strain of claim 43, wherein said metabolic enzyme is an alanine racemase enzyme or a D-amino acid transferase enzyme.

47. (canceled)

48. The recombinant Listeria strain of claim 31, wherein said recombinant Listeria strain has been passaged through an animal host.

49. The recombinant Listeria strain of claim 31, wherein said recombinant Listeria strain is a recombinant Listeria monocytogenes strain.

50. A vaccine comprising the recombinant Listeria strain of claim 31 and an adjuvant, cytokine, chemokine, or combination thereof.

51. A recombinant Listeria strain comprising at least one episomal recombinant nucleic acid molecule, said nucleic acid molecule comprising a first and at least a second open reading frame each encoding a first and at least a second polypeptide, wherein said first and said at least second polypeptide each comprise a heterologous antigen or a functional fragment thereof fused to an N-terminal truncated LLO polypeptide, an N-terminal ActA polypeptide, or PEST-peptide, or a functional fragment thereof, and wherein said nucleic acid further comprises an open reading frame encoding a plasmid replication control region.

52. The recombinant Listeria strain of claim 51, wherein said at least one episomal recombinant nucleic acid further comprises a third open reading frame encoding a third polypeptide, wherein said third polypeptide comprises a heterologous antigen or a functional fragment thereof fused to an N-terminal truncated LLO polypeptide, an N-terminal ActA polypeptide, or PEST-peptide, or a functional fragment thereof.

53. The recombinant Listeria strain of claim 51, wherein said plasmid replication control region enables the control of expression of exogenous heterologous antigenic polypeptide from each of said first or said at least second nucleic acid molecules.

54. The recombinant Listeria strain of claim 52, wherein said plasmid replication control region enables the control of expression of exogenous heterologous antigenic polypeptide from each of said first, second or third nucleic acid molecules.

55. (canceled)

56. (canceled)

57. (canceled)

58. (canceled)

59. The recombinant Listeria strain of claim 51, wherein said recombinant Listeria comprises up to four episomal recombinant nucleic acid molecules, each comprising a first and at least a second open reading frame, wherein each of said first and at least second open reading frame encode a first polypeptide and at least a second polypeptide, wherein said first and said at least second polypeptide each comprise a heterologous antigen or a functional fragment thereof fused to an endogenous PEST-containing polypeptide, and wherein each of said recombinant nucleic acid further comprise an open reading frame encoding said plasmid replication control region.

60. The recombinant Listeria of claim 59, wherein each of said plasmid replication control region enables the control of expression of each episomal recombinant nucleic acid copy number to 3 or 4 copies per Listeria.

61. (canceled)

62. (canceled)

63. The recombinant Listeria strain of claim 51, wherein said first, or said at least second heterologous antigen is expressed by a tumor cell.

64. The recombinant Listeria strain of claim 51, wherein said first, or said at least second polypeptide comprises an angiogenic antigen or an antigen associated with tumor evasion or resistance to cancer or an antigen associated with the local tissue environment that is further associated with the development or metastasis of cancer.

65. (canceled)

66. (canceled)

67. (canceled)

68. The recombinant Listeria strain of claim 52, wherein said recombinant Listeria strain is an auxotrophic Listeria strain, comprising an episomal expression vector comprising a metabolic enzyme that complements the auxotrophy of said auxotrophic Listeria strain.

69. The recombinant Listeria strain of claim 68, wherein said auxotrophic Listeria strain is a dal/dat mutant.

70. (canceled)

71. The recombinant Listeria strain of claim 70, wherein said metabolic enzyme is an amino acid metabolism enzyme.

72. (canceled)

73. The recombinant Listeria strain of claim 70, wherein said metabolic enzyme is an alanine racemase enzyme or a D-amino acid transferase enzyme.

74. (canceled)

75. The recombinant Listeria strain of claim 51, wherein said recombinant Listeria strain has been passaged through an animal host.

76. The recombinant Listeria strain of claim 51, wherein said recombinant Listeria strain is a recombinant Listeria monocytogenes strain.

77. A vaccine comprising the recombinant Listeria strain of claim 51 and an adjuvant, cytokine, chemokine, or combination thereof.

78. A method of inducing an immune response to an antigen in a subject comprising administering to said subject a composition comprising a recombinant Listeria strain comprising at least one episomal recombinant nucleic acid molecule, said nucleic acid molecule comprising a first and at least a second open reading frame each encoding a first and at least a second polypeptide, and wherein said first and said at least second polypeptide each comprise a heterologous antigen or a functional fragment thereof fused to an N-terminal truncated LLO polypeptide, an N-terminal ActA polypeptide, or PEST-peptide, or a functional fragment thereof.

79. The method of claim 78, wherein said at least one episomal recombinant nucleic acid further comprises a third open reading frame encoding a third polypeptide, wherein said third polypeptide comprises a heterologous antigen or a functional fragment thereof fused to an N-terminal truncated LLO polypeptide, an N-terminal ActA polypeptide, or PEST-peptide, or a functional fragment thereof.

80. (canceled)

81. (canceled)

82. The method of claim 78, wherein said first, or said at least second heterologous antigen is expressed by a tumor cell.

83. The method of claim 78, wherein said first, or said at least second polypeptide comprises an angiogenic antigen or an antigen associated with tumor evasion or resistance to cancer or an antigen is associated with the local tissue environment that is further associated with the development or metastasis of cancer.

84. (canceled)

85. (canceled)

86. (canceled)

87. The method of claim 79, wherein said recombinant Listeria strain is an auxotrophic Listeria strain, comprising a metabolic enzyme that complements the auxotrophy of said auxotrophic Listeria strain.

88. The method of claim 87, wherein said auxotrophic Listeria strain is a dal/dat mutant.

89. (canceled)

90. The method of claim 89, wherein said metabolic enzyme is an amino acid metabolism enzyme.

91. (canceled)

92. The method of claim 89, wherein said metabolic enzyme is an alanine racemase enzyme or a D-amino acid transferase enzyme.

93. (canceled)

94. The method of claim 78, wherein said recombinant Listeria strain has been passaged through an animal host.

95. The method of claim 78, wherein said recombinant Listeria strain is a recombinant Listeria monocytogenes strain.

96. The method of claim 78, wherein said recombinant Listeria strain is administered with an adjuvant, cytokine, chemokine, or combination thereof.

97. A method of treating, suppressing, or inhibiting a cancer in a subject comprising administering a recombinant Listeria strain of any one of claim 11, 31, or 51 to said subject.

98. (canceled)

99. A method of producing a recombinant Listeria strain comprising an episomal expression plasmid comprising a first and at least a second nucleic acid encoding a first and at least a second polypeptide, wherein said first and said at least second polypeptide each comprise a heterologous antigen fused to an endogenous PEST-containing polypeptide, said method comprising the steps of:
(a) recombinantly fusing in said plasmid said first and said at least second nucleic acid encoding said first and said second polypeptide each comprising a first and a second heterologous antigen fused to an endogenous PEST-containing polypeptide;
(b) transforming said recombinant Listeria with said episomal expression plasmid; and,
(c) expressing said first, and said at least second antigens under conditions conducive to antigenic expression in said recombinant Listeria strain.

100. The method of claim 99 wherein said episomal expression plasmid further comprises a third polypeptide comprising a heterologous antigen fused to an endogenous PEST-containing polypeptide, wherein said method further comprises the steps of:
(a) recombinantly fusing in said plasmid said third nucleic acid encoding said third polypeptide comprising a third heterologous antigen fused to an endogenous PEST-containing polypeptide;
(b) transforming said recombinant Listeria with said episomal expression plasmid; and,
(c) expressing said first, said second and said third antigens under conditions conducive to antigenic expression in said recombinant Listeria strain.

101. A method of producing a recombinant Listeria strain comprising an integrated first nucleic acid, and an episomal expression plasmid comprising a second, and a third nucleic acid each encoding a first, a second, and a third polypeptide, wherein said first, second and third polypeptides each comprise a heterologous antigen fused to an endogenous PEST-containing polypeptide, the method comprising the steps of:
(a) integrating said first nucleic acid encoding said first polypeptide comprising a first heterologous antigen fused to an endogenous PEST-containing polypeptide into said recombinant Listeria's genome;
(b) recombinantly fusing in said plasmid said second and said third nucleic acid encoding said second and said third polypeptide each comprising a second and a third heterologous antigen fused to an endogenous PEST-containing polypeptide;
(c) transforming said recombinant Listeria with said episomal expression plasmid; and,
(d) expressing said first, second, and third antigens under conditions conducive to antigenic expression in said recombinant Listeria strain.

102. The method of claim 100, wherein said episomal expression plasmid further comprises a plasmid replication control region,
(c) wherein if the expression of said first, said second and said third antigens place a metabolic burden on said Listeria, said plasmid's replication control region activates and expresses a repressor that represses plasmid replication and represses expression of the first, second, and the third heterologous antigen or fragment thereof from each plasmid.

103. The method of claim 102, wherein said recombinant Listeria comprises up to four episomal expression plasmids, each comprising a first, a second, and a third open reading frame encoding said first, said second and said third, wherein said first, said second, and said third polypeptide each comprise a heterologous antigen or a functional fragment thereof fused to an endogenous PEST-containing polypeptide, and wherein each of said recombinant nucleic acids further comprise an open reading frame encoding said plasmid replication control region.

104. The method of claim 103, wherein each of said plasmid replication control region enables the control of expression of each episomal expression plasmid copy number to 3 or 4 copies per Listeria.

105. (canceled)

106. (canceled)

107. (canceled)

108. The method of claim 101, wherein said episomal expression plasmid further comprises a plasmid replication control region,
(d) wherein if the expression of said first, said second, and said third antigens place a metabolic burden on said Listeria, said plasmid's replication control region activates and expresses a repressor that represses plasmid replication and represses expression of the first, second, and the third heterologous antigen or fragment thereof from each plasmid.

109. The method of claim 108, wherein said recombinant Listeria comprises up to four episomal expression plasmid, each comprising a first, a second, and a third open reading frame, wherein each of said first, second, and third open reading frame encode a first polypeptide, a second polypeptide, and a third polypeptide, wherein said first, said second, and said third polypeptide each comprise a heterologous antigen or a functional fragment thereof fused to an endogenous PEST-containing polypeptide, and wherein each of said recombinant nucleic acids further comprise an open reading frame encoding said plasmid replication control region.

110. The method of claim 109, wherein each of said plasmid replication control region enables the control of expression of each episomal expression plasmid copy number to 3 or 4 copies per Listeria.


SEQUENCE LISTING (35 sequences)

SEQ ID NO: 1 - protein, 32 aa, Listeria monocytogenes
KENSISSMAPPASPPASPKTPIEKKHADEIDK

SEQ ID NO: 2 - protein, 19 aa, Listeria monocytogenes
KENSISSMAPPASPPASPK

SEQ ID NO: 3 - protein, 14 aa, Listeria monocytogenes
KTEEQPSEVNTGPR

SEQ ID NO: 4 - protein, 28 aa, Listeria monocytogenes
KASVTDTSEGDLDSSMQSADESTPQPLK

SEQ ID NO: 5 - protein, 20 aa, Listeria monocytogenes
KNEEVNASDFPPPPTDEELR

SEQ ID NO: 6 - protein, 33 aa, Listeria monocytogenes
RGGIPTSEEFSSLNSGDFTDDENSETTEEEIDR

SEQ ID NO: 7 - protein, 19 aa, Listeria monocytogenes
RSEVTISPAETPESPPATP

SEQ ID NO: 8 - protein, 17 aa, Streptococcus pyogenes
KQNTASTETTTTNEQPK

SEQ ID NO: 9 - protein, 17 aa, Streptococcus equisimilis
KQNTANTETTTTNEQPK

SEQ ID NO: 10 - protein, 441 aa, Artificial Sequence
  N-terminal LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTDGKINIDHSGGYVAQFNISWDEVNYD

SEQ ID NO: 11 - protein, 416 aa, Artificial Sequence
  N-terminal LLO fragment
MKKIMLVFITLILVSLPIAQQTEAKDASAFNKENSISSVAPPASPPASPKTPIEKKHADEIDKYIQGLDYNKNNVLVYHGDAVTNVPPRKGYKDGNEYIVVEKKKKSINQNNADIQVVNAISSLTYPGALVKANSELVENQPDVLPVKRDSLTLSIDLPGMTNQDNKIVVKNATKSNVNNAVNTLVERWNEKYAQAYSNVSAKIDYDDEMAYSESQLIAKFGTAFKAVNNSLNVNFGAISEGKMQEEVISFKQIYYNVNVNEPTRPSRFFGKAVTKEQLQALGVNAENPPAYISSVAYGRQVYLKLSTNSHSTKVKAAFDAAVSGKSVSGDVELTNIIKNSSFKAVIYGGSAKDEVQIIDGNLGDLRDILKKGATFNRETPGVPIAYTTNFLKDNELAVIKNNSEYIETTSKAYTD

SEQ ID NO: 12 - protein, 416 aa, Artificial Sequence; identical to SEQ ID NO: 11
  N-terminal LLO fragment

SEQ ID NO: 13 - protein, 390 aa, Artificial Sequence
  ActA fragment
MRAMMVVFITANCITINPDIIFAATDSEDSSLNTDEWEEEKTEEQPSEVNTGPRYETAREVSSRDIKELEKSNKVRNTNKADLIAMLKEKAEKGPNINNNNSEQTENAAINEEASGADRPAIQVERRHPGLPSDSAAEIKKRRKAIASSDSELESLTYPDKPTKVNKKKVAKESVADASESDLDSSMQSADESSPQPLKANQQPFFPKVFKKIKDAGKWVRDKIDENPEVKKAIVDKSAGLIDQLLTKKKSEEVNASDFPPPPTDEELRLALPETPMLLGFNAPATSEPSSFEFPPPPTDEELRLALPETPMLLGFNAPATSEPSSFEFPPPPTEDELEIIRETASSLDSSFTRGDLASLRNAINRHSQNFSDFPPIPTEEELNGRGGRP

SEQ ID NO: 14 - DNA, 1170 bp, Artificial Sequence
  ActA fragment
atgcgtgcgatgatggtggttttcattactgccaattgcattacgattaaccccgacataatatttgcagcgacagatagcgaagattctagtctaaacacagatgaatgggaagaagaaaaaacagaagagcaaccaagcgaggtaaatacgggaccaagatacgaaactgcacgtgaagtaagttcacgtgatattaaagaactagaaaaatcgaataaagtgagaaatacgaacaaagcagacctaatagcaatgttgaaagaaaaagcagaaaaaggtccaaatatcaataataacaacagtgaacaaactgagaatgcggctataaatgaagaggcttcaggagccgaccgaccagctatacaagtggagcgtcgtcatccaggattgccatcggatagcgcagcggaaattaaaaaaagaaggaaagccatagcatcatcggatagtgagcttgaaagccttacttatccggataaaccaacaaaagtaaataagaaaaaagtggcgaaagagtcagttgcggatgcttctgaaagtgacttagattctagcatgcagtcagcagatgagtcttcaccacaacctttaaaagcaaaccaacaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccaatgcttcttggttttaatgctcctgctacatcagaaccgagctcattcgaatttccaccaccacctacggatgaagagttaagacttgctttgccagagacgccaatgcttcttggttttaatgctcctgctacatcggaaccgagctcgttcgaatttccaccgcctccaacagaagatgaactagaaatcatccgggaaacagcatcctcgctagattctagttttacaagaggggatttagctagtttgagaaatgctattaatcgccatagtcaaaatttctctgatttcccaccaatcccaacagaagaagagttgaacgggagaggcggtagacca

SEQ ID NO: 15 - protein, 390 aa, Artificial Sequence
  ActA fragment
MRAMMVVFITANCITINPDIIFAATDSEDSSLNTDEWEEEKTEEQPSEVNTGPRYETAREVSSRDIEELEKSNKVKNTNKADLIAMLKAKAEKGPNNNNNNGEQTGNVAINEEASGVDRPTLQVERRHPGLSSDSAAEIKKRRKAIASSDSELESLTYPDKPTKANKRKVAKESVVDASESDLDSSMQSADESTPQPLKANQKPFFPKVFKKIKDAGKWVRDKIDENPEVKKAIVDKSAGLIDQLLTKKKSEEVNASDFPPPPTDEELRLALPETPMLLGFNAPTPSEPSSFEFPPPPTDEELRLALPETPMLLGFNAPATSEPSSFEFPPPPTEDELEIMRETAPSLDSSFTSGDLASLRSAINRHSENFSDFPLIPTEEELNGRGGRP

SEQ ID NO: 16 - DNA, 1170 bp, Artificial Sequence
  ActA fragment
atgcgtgcgatgatggtagttttcattactgccaactgcattacgattaaccccgacataatatttgcagcgacagatagcgaagattccagtctaaacacagatgaatgggaagaagaaaaaacagaagagcagccaagcgaggtaaatacgggaccaagatacgaaactgcacgtgaagtaagttcacgtgatattgaggaactagaaaaatcgaataaagtgaaaaatacgaacaaagcagacctaatagcaatgttgaaagcaaaagcagagaaaggtccgaataacaataataacaacggtgagcaaacaggaaatgtggctataaatgaagaggcttcaggagtcgaccgaccaactctgcaagtggagcgtcgtcatccaggtctgtcatcggatagcgcagcggaaattaaaaaaagaagaaaagccatagcgtcgtcggatagtgagcttgaaagccttacttatccagataaaccaacaaaagcaaataagagaaaagtggcgaaagagtcagttgtggatgcttctgaaagtgacttagattctagcatgcagtcagcagacgagtctacaccacaacctttaaaagcaaatcaaaaaccatttttccctaaagtatttaaaaaaataaaagatgcggggaaatgggtacgtgataaaatcgacgaaaatcctgaagtaaagaaagcgattgttgataaaagtgcagggttaattgaccaattattaaccaaaaagaaaagtgaagaggtaaatgcttcggacttcccgccaccacctacggatgaagagttaagacttgctttgccagagacaccgatgcttctcggttttaatgctcctactccatcggaaccgagctcattcgaatttccgccgccacctacggatgaagagttaagacttgctttgccagagacgccaatgcttcttggttttaatgctcctgctacatcggaaccgagctcattcgaatttccaccgcctccaacagaagatgaactagaaattatgcgggaaacagcaccttcgctagattctagttttacaagcggggatttagctagtttgagaagtgctattaatcgccatagcgaaaatttctctgatttcccactaatcccaacagaagaagagttgaacgggagaggcggtagacca

SEQ ID NO: 17 - DNA, 1256 bp, Artificial Sequence
  ActA fragment
gcgccaaatcattggttgattggtgaggatgtctgtgtgcgtgggtcgcgagatgggcgaataagaagcattaaagatcctgacaaatataatcaagcggctcatatgaaagattacgaatcgcttccactcacagaggaaggcgactggggcggagttcattataatagtggtatcccgaataaagcagcctataatactatcactaaacttggaaaagaaaaaacagaacagctttattttcgcgccttaaagtactatttaacgaaaaaatcccagtttaccgatgcgaaaaaagcgcttcaacaagcagcgaaagatttatatggtgaagatgcttctaaaaaagttgctgaagcttgggaagcagttggggttaactgattaacaaatgttagagaaaaattaattctccaagtgatattcttaaaataattcatgaatattttttcttatattagctaattaagaagataactaactgctaatccaatttttaacggaacaaattagtgaaaatgaaggccgaattttccttgttctaaaaaggttgtattagcgtatcacgaggagggagtataagtgggattaaacagatttatgcgtgcgatgatggtggttttcattactgccaattgcattacgattaaccccgacgtcgacccatacgacgttaattcttgcaatgttagctattggcgtgttctctttaggggcgtttatcaaaattattcaattaagaaaaaataattaaaaacacagaacgaaagaaaaagtgaggtgaatgatatgaaattcaaaaaggtggttctaggtatgtgcttgatcgcaagtgttctagtctttccggtaacgataaaagcaaatgcctgttgtgatgaatacttacaaacacccgcagctccgcatgatattgacagcaaattaccacataaacttagttggtccgcggataacccgacaaatactgacgtaaatacgcactattggctttttaaacaagcggaaaaaatactagctaaagatgtaaatcatatgcgagctaatttaatgaatgaacttaaaaaattcgataaacaaatagctcaaggaatatatgatgcggatcataaaaatccatattatgatactagtacatttttatctcatttttataatcctgatagagataatacttatttgccgggttttgctaatgcgaaaataacaggagcaaagtatttcaatcaatcggtgactgattaccgagaagggaa

SEQ ID NO: 18 - protein, 2322 aa, Homo sapiens [sequence omitted]

SEQ ID NO: 19 - DNA, 7011 bp, Homo sapiens
atgcagtccggccgcggccccccacttccagcccccggcctggccttggctttgaccctgactatgttggccagacttgcatccgcggcttccttcttcggtgagaaccacctggaggtgcctgtggccacggctctgaccgacatagacctgcagctgcagttctccacgtcccagcccgaagccctccttctcctggcagcaggcccagctgaccacctcctgctgcagctctactctggacgcctgcaggtcagacttgttctgggccaggaggagctgaggctgcagactccagcagagacgctgctgagtgactccatcccccacactgtggtgctgactgtcgtagagggctgggccacgttgtcagtcgatgggtttctgaacgcctcctcagcagtcccaggagcccccctagaggtcccctatgggctctttgttgggggcactgggacccttggcctgccctacctgaggggaaccagccgacccctgaggggttgcctccatgcagccaccctcaatggccgcagcctcctccggcctctgacccccgatgtgcatgagggctgtgctgaagagttttctgccagtgatgatgtggccctgggcttctctgggccccactctctggctgccttccctgcctggggcactcaggacgaaggaaccctagagtttacactcaccacacagagccggcaggcacccttggccttccaggcagggggccggcgtggggacttcatctatgtggacatatttgagggccacctgcgggccgtggtggagaagggccagggtaccgtattgctccacaacagtgtgcctgtggccgatgggcagccccatgaggtcagtgtccacatcaatgctcaccggctggaaatctccgtggaccagtaccctacgcatacttcgaaccgaggagtcctcagctacctggagccacggggcagtctccttctcggggggctggatgcagaggcctctcgtcacctccaggaacaccgcctgggcctgacaccagaggccaccaatgcctccctgctgggctgcatggaagacctcagtgtcaatggccagaggcgggggctgcgggaagctttgctgacgcgcaacatggcagccggctgcaggctggaggaggaggagtatgaggacgatgcctatggacattatgaagctttctccaccctggcccctgaggcttggccagccatggagctgcctgagccatgcgtgcctgagccagggctgcctcctgtctttgccaatttcacccagctgctgactatcagcccactggtggtggccgaggggggcacagcctggcttgagtggaggcatgtgcagcccacgctggacctgatggaggctgagctgcgcaaatcccaggtgctgttcagcgtgacccgaggggcacgccatggcgagctcgagctggacatcccgggagcccaggcacgaaaaatgttcaccctcctggacgtggtgaaccgcaaggcccgcttcatccacgatggctctgaggacacctccgaccagctggtgctggaggtgtcggtgacggctcgggtgcccatgccctcatgccttcggaggggccaaacatacctcctgcccatccaggtcaaccctgtcaatgacccaccccacatcatcttcccacatggcagcctcatggtgatcctggaacacacgcagaagccgctggggcctgaggttttccaggcctatgacccggactctgcctgtgagggcctcaccttccaggtccttggcacctcctctggcctccccgtggagcgccgagaccagcctggggagccggcgaccgagttctcctgccgggagttggaggccggcagcctagtctatgtccaccgcggtggtcctgcacaggacttgacgttccgggtcagcgatggactgcaggccagccccccggccacgctgaaggtggtggccatccggccggccatacagatccaccgcagcacagggttgcgactggcccaaggctctgccatgcccatcttgcccgccaacctgtcggtggagaccaatgccgtggggcaggatgtgagcgtgctgttccgcgtcactggggccctgcagtttggggagctgcagaagcagggggcaggtggggtggagggtgctgagtggtgggccacacaggcgttccaccagcgggatgtggagcagggccgcgtgaggtacctgagcactgacccacagcaccacgcttacgacaccgtggagaacctggccctggaggtgcaggtgggccaggagatcctgagcaatctgtccttcccagtgaccatccagagagccactgtgtggatgctgcggctggagccactgcacactcagaacacccagcaggagaccctcaccacagcccacctggaggccaccctggaggaggcaggcccaagccccccaaccttccattatgaggtggttcaggctcccaggaaaggcaaccttcaactacagggcacaaggctgtcagatggccagggcttcacccaggatgacatacaggctggccgggtgacctatggggccacagcacgtgcctcagaggcagtcgaggacaccttccgtttccgtgtcacagctccaccatatttctccccactctataccttccccatccacattggtggtgacccagatgcgcctgtcctcaccaatgtcctcctcgtggtgcctgagggtggtgagggtgtcctctctgctgaccacctctttgtcaagagtctcaacagtgccagctacctctatgaggtcatggagcggccccgccatgggaggttggcttggcgtgggacacaggacaagaccactatggtgacatccttcaccaatgaagacctgttgcgtggccggctggtctaccagcatgatgactccgagaccacagaagatgatatcccatttgttgctacccgccagggcgagagcagtggtgacatggcctgggaggaggtacggggtgtcttccgagtggccatccagcccgtgaatgaccacgcccctgtgcagaccatcagccggatcttccatgtggcccggggtgggcggcggctgctgactacagacgacgtggccttcagcgatgctgactcgggctttgctgacgcccagctggtgcttacccgcaaggacctcctctttggcagtatcgtggccgtagatgagcccacgcggcccatctaccgcttcacccaggaggacctcaggaagaggcgagtactgttcgtgcactcaggggctgaccgtggctggatccagctgcaggtgtccgacgggcaacaccaggccactgcgctgctggaggtgcaggcctcggaaccctacctccgtgtggccaacggctccagccttgtggtccctcaagggggccagggcaccatcgacacggccgtgctccacctggacaccaacctcgacatccgcagtggggatgaggtccactaccacgtcacagctggccctcgctggggacagctagtccgggctggtcagccagccacagccttctcccagcaggacctgctggatggggccgttctctatagccacaatggcagcctcagcccccgcgacaccatggccttctccgtggaagcagggccagtgcacacggatgccaccctacaagtgaccattgccctagagggcccactggccccactgaagctggtccggcacaagaagatctacgtcttccagggagaggcagctgagatcagaagggaccagctggaggcagcccaggaggcagtgccacctgcagacatcgtattctcagtgaagagcccaccgagtgccggctacctggtgatggtgtcgcgtggcgccttggcagatgagccacccagcctggaccctgtgcagagcttctcccaggaggcagtggacacaggcagggtcctgtacctgcactcccgccctgaggcctggagcgatgccttctcgctggatgtggcctcaggcctgggtgctcccctcgagggcgtccttgtggagctggaggtgctgcccgctgccatcccactagaggcgcaaaacttcagcgtccctgagggtggcagcctcaccctggcccctccactgctccgtgtctccgggccctacttccccactctcctgggcctcagcctgcaggtgctggagccaccccagcatggagccctgcagaaggaggacggacctcaagccaggaccctcagcgccttctcctggagaatggtggaagagcagctgatccgctacgtgcatgacgggagcgagacactgacagacagttttgtcctgatggctaatgcctccgagatggatcgccagagccatcctgtggccttcactgtcactgtcctgcctgtcaatgaccaaccccccatcctcactacaaacacaggcctgcagatgtgggagggggccactgcgcccatccctgcggaggctctgaggagcacggacggcgactctgggtctgaggatctggtctacaccatcgagcagcccagcaacgggcgggtagtgctgcggggggcgccgggcactgaggtgcgcagcttcacgcaggcccagctggacggcgggctcgtgctgttctcacacagaggaaccctggatggaggcttccgcttccgcctctctgacggcgagcacacttcccccggacacttcttccgagtgacggcccagaagcaagtgctcctctcgctgaagggcagccagacactgactgtctgcccagggtccgtccagccactcagcagtcagaccctcagggccagctccagcgcaggcactgacccccagctcctgctctaccgtgtggtgcggggcccccagctaggccggctgttccacgcccagcaggacagcacaggggaggccctggtgaacttcactcaggcagaggtctacgctgggaatattctgtatgagcatgagatgccccccgagcccttttgggaggcccatgataccctagagctccagctgtcctcgccgcctgcccgggacgtggccgccacccttgctgtggctgtgtcttttgaggctgcctgtccccagcgccccagccacctctggaagaacaaaggtctctgggtccccgagggccagcgggccaggatcaccgtggctgctctggatgcctccaatctcttggccagcgttccatcaccccagcgctcagagcatgatgtgctcttccaggtcacacagttccccagccggggccagctgttggtgtccgaggagcccctccatgctgggcagccccacttcctgcagtcccagctggctgcagggcagctagtgtatgcccacggcggtgggggcacccagcaggatggcttccactttcgtgcccacctccaggggccagcaggggcctccgtggctggaccccaaacctcagaggcctttgccatcacggtgagggatgtaaatgagcggccccctcagccacaggcctctgtcccactccggctcacccgaggctctcgtgcccccatctcccgggcccagctgagtgtggtggacccagactcagctcctggggagattgagtacgaggtccagcgggcaccccacaacggcttcctcagcctggtgggtggtggcctggggcccgtgacccgcttcacgcaagccgatgtggattcagggcggctggccttcgtggccaacgggagcagcgtggcaggcatcttccagctgagcatgtctgatggggccagcccacccctgcccatgtccctggctgtggacatcctaccatccgccatcgaggtgcagctgcgggcacccctggaggtgccccaagctttggggcgctcctcactgagccagcagcagctccgggtggtttcagatcgggaggagccagaggcagcataccgcctcatccagggaccccagtatgggcatctcctggtgggcgggcggcccacctcggccttcagccaattccagatagaccagggcgaggtggtctttgccttcaccaacttctcctcctctcatgaccacttcagagtcctggcactggctaggggtgtcaatgcatcagccgtagtgaacgtcactgtgagggctctgctgcatgtgtgggcaggtgggccatggccccagggtgccaccctgcgcctggaccccaccgtcctagatgctggcgagctggccaaccgcacaggcagtgtgccgcgcttccgcctcctggagggaccccggcatggccgcgtggtccgcgtgccccgagccaggacggagcccgggggcagccagctggtggagcagttcactcagcaggaccttgaggacgggaggctggggctggaggtgggcaggccagaggggagggcccccggccccgcaggtgacagtctcactctggagctgtgggcacagggcgtcccgcctgctgtggcctccctggactttgccactgagccttacaatgctgcccggccctacagcgtggccctgctcagtgtccccgaggccgcccggacggaagcagggaagccagagagcagcacccccacaggcgagccaggccccatggcatccagccctgagcccgctgtggccaagggaggcttcctgagcttccttgaggccaacatgttcagcgtcatcatccccatgtgcctggtacttctgctcctggcgctcatcctgcccctgctcttctacctccgaaaacgcaacaagacgggcaagcatgacgtccaggtcctgactgccaagccccgcaacggcctggctggtgacaccgagacctttcgcaaggtggagccaggccaggccatcccgctcacagctgtgcctggccaggggccccctccaggaggccagcctgacccagagctgctgcagttctgccggacacccaaccctgcccttaagaatggccagtactgggtgtgaggcctggcctgggcccagatgctgatcgggccagggacaggc

SEQ ID NO: 20 - protein, 24 aa, Homo sapiens
MWVPVVFLTLSVTWIGAAPLILSR

SEQ ID NO: 21 - DNA, 6523 bp, Artificial Sequence
  nucleic acid sequence of plasmid pAdv142
cggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagattgtgggaggctgggagtgcgagaagcattcccaaccctggcaggtgcttgtggcctctcgtggcagggcagtctgcggcggtgttctggtgcacccccagtgggtcctcacagctgcccactgcatcaggaacaaaagcgtgatcttgctgggtcggcacagcctgtttcatcctgaagacacaggccaggtatttcaggtcagccacagcttcccacacccgctctacgatatgagcctcctgaagaatcgattcctcaggccaggtgatgactccagccacgacctcatgctgctccgcctgtcagagcctgccgagctcacggatgctgtgaaggtcatggacctgcccacccaggagccagcactggggaccacctgctacgcctcaggctggggcagcattgaaccagaggagttcttgaccccaaagaaacttcagtgtgtggacctccatgttatttccaatgacgtgtgtgcgcaagttcaccctcagaaggtgaccaagttcatgctgtgtgctggacgctggacagggggcaaaagcacctgctcgggtgattctgggggcccacttgtctgttatggtgtgcttcaaggtatcacgtcatggggcagtgaaccatgtgccctgcccgaaaggccttccctgtacaccaaggtggtgcattaccggaagtggatcaaggacaccatcgtggccaacccctaacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcgcggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaattaa

SEQ ID NO: 22 - DNA, 36 bp, Artificial Sequence
  Adv271-actAF1 primer
cggaattcggatccgcgccaaatcattggttgattg

SEQ ID NO: 23 - DNA, 37 bp, Artificial Sequence
  Adv272-actAR1 primer
gcgagtcgacgtcggggttaatcgtaatgcaattggc

SEQ ID NO: 24 - DNA, 35 bp, Artificial Sequence
  Adv273-actAF2 primer
gcgagtcgacccatacgacgttaattcttgcaatg

SEQ ID NO: 25 - DNA, 39 bp, Artificial Sequence
  Adv274-actAR2 primer
gatactgcagggatccttcccttctcggtaatcagtcac

SEQ ID NO: 26 - DNA, 30 bp, Artificial Sequence
  primers ActA-F primer
atcccgggtgaagcttgggaagcagttggg

SEQ ID NO: 27 - DNA, 32 bp, Artificial Sequence
  primers ActA-R primer
attctagatttatcacgtacccatttccccgc

SEQ ID NO: 28 - DNA, 32 bp, Artificial Sequence
  Ch-Her2-F  primer
attctagaacccacctggacatgctccgccac

SEQ ID NO: 29 - DNA, 67 bp, Artificial Sequence
  Ch-Her2-R primer
gtcgacactagtctagtggtgatggtgatgatggagctcagatctgtctaagaggcagccatagggc

SEQ ID NO: 30 - DNA, 6733 bp, Artificial Sequence
  nucleic acid sequence of plasmid backbone
ggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagactagttctagatttatcacgtacccatttccccgcatcttttatttttttaaatactttagggaaaaatggtttttgatttgcttttaaaggttgtggtgtagactcgtctgctgactgcatgctagaatctaagtcactttcagaagcatccacaactgactctttcgccacttttctcttatttgcttttgttggtttatctggataagtaaggctttcaagctcactatccgacgacgctatggcttttcttctttttttaatttccgctgcgctatccgatgacagacctggatgacgacgctccacttgcagagttggtcggtcgactcctgaagcctcttcatttatagccacatttcctgtttgctcaccgttgttattattgttattcggacctttctctgcttttgctttcaacattgctattaggtctgctttgttcgtatttttcactttattcgatttttctagttcctcaatatcacgtgaacttacttcacgtgcagtttcgtatcttggtcccgtatttacctcgcttggctgctcttctgttttttcttcttcccattcatctgtgtttagactggaatcttcgctatctgtcgctgcaaatattatgtcggggttaatcgtaatgcagttggcagtaatgaaaactaccatcatcgcacgcataaatctgtttaatcccacttatactccctcctcgtgatacgctaatacaacctttttagaacaaggaaaattcggccttcattttcactaatttgttccgttaaaaattggattagcagttagttatcttcttaattagctaatataagaaaaaatattcatgaattattttaagaatatcacttggagaattaatttttctctaacatttgttaatcagttaaccccaactgcttcccaagcttcacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaat

SEQ ID NO: 31 - DNA, 8317 bp, Artificial Sequence
  nucleic acid sequence of plasmid and antigens
ggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagccctcctttgattagtatattcctatcttaaagttacttttatgtggaggcattaacatttgttaatgacgtcaaaaggatagcaagactagaataaagctataaagcaagcatataatattgcgtttcatctttagaagcgaatttcgccaatattataattatcaaaagagaggggtggcaaacggtatttggcattattaggttaaaaaatgtagaaggagagtgaaacccatgaaaaaaataatgctagtttttattacacttatattagttagtctaccaattgcgcaacaaactgaagcaaaggatgcatctgcattcaataaagaaaattcaatttcatccatggcaccaccagcatctccgcctgcaagtcctaagacgccaatcgaaaagaaacacgcggatgaaatcgataagtatatacaaggattggattacaataaaaacaatgtattagtataccacggagatgcagtgacaaatgtgccgccaagaaaaggttacaaagatggaaatgaatatattgttgtggagaaaaagaagaaatccatcaatcaaaataatgcagacattcaagttgtgaatgcaatttcgagcctaacctatccaggtgctctcgtaaaagcgaattcggaattagtagaaaatcaaccagatgttctccctgtaaaacgtgattcattaacactcagcattgatttgccaggtatgactaatcaagacaataaaatagttgtaaaaaatgccactaaatcaaacgttaacaacgcagtaaatacattagtggaaagatggaatgaaaaatatgctcaagcttatccaaatgtaagtgcaaaaattgattatgatgacgaaatggcttacagtgaatcacaattaattgcgaaatttggtacagcatttaaagctgtaaataatagcttgaatgtaaacttcggcgcaatcagtgaagggaaaatgcaagaagaagtcattagttttaaacaaatttactataacgtgaatgttaatgaacctacaagaccttccagatttttcggcaaagctgttactaaagagcagttgcaagcgcttggagtgaatgcagaaaatcctcctgcatatatctcaagtgtggcgtatggccgtcaagtttatttgaaattatcaactaattcccatagtactaaagtaaaagctgcttttgatgctgccgtaagcggaaaatctgtctcaggtgatgtagaactaacaaatatcatcaaaaattcttccttcaaagccgtaatttacggaggttccgcaaaagatgaagttcaaatcatcgacggcaacctcggagacttacgcgatattttgaaaaaaggcgctacttttaatcgagaaacaccaggagttcccattgcttatacaacaaacttcctaaaagacaatgaattagctgttattaaaaacaactcagaatatattgaaacaacttcaaaagcttatacagatggaaaaattaacatcgatcactctggaggatacgttgctcaattcaacatttcttgggatgaagtaaattatgatctcgagcatggagatacacctacattgcatgaatatatgttagatttgcaaccagagacaactgatctctactgttatgagcaattaaatgacagctcagaggaggaggatgaaatagatggtccagctggacaagcagaaccggacagagcccattacaatattgtaaccttttgttgcaagtgtgactctacgcttcggttgtgcgtacaaagcacacacgtagacattcgtactttggaagacctgttaatgggcacactaggaattgtgtgccccatctgttctcagaaaccataaactagtctagtggtgatggtgatgatggagctcagatctgtctaagaggcagccatagggcataagctgtgtcaccagctgcaccgtggatgtcaggcagatgcccagaaggcgggagacatatggggagcccacaccagccatcacgtatgcttcgtctaagatttctttgttggctttgggggatgtgttttccctcaacactttgatggccactggaattttcacattctccccatcagggatccagatgcccttgtagactgtgccaaaagcgccagatccaagcaccttcaccttcctcagctccgtctctttcaggatccgcatctgcgcctggttgggcatcgctccgctaggtgtcagcggctccaccagctccgtttcctgcagcagtctccgcatcgtgtacttccggatcttctgctgccctcgggcgcacagctggtggcaggccaggccctcgcccacacactcgtcctctggccggttggcagtgtggagcagagcttggtgcgggttccgaaagagctggtcccagggcaccgtgtgcacgaagcagaggtgggtgttatggtggatgagggccagtccactgcccagttccctcagtgagcgcagccccagccagctgatgcccagcccttgcagggtcagcgagtaggcgccattgtgcagaattcgtccccggattacttgcaggttctggaagacgctgaggtcaggcaggctgtccggccatgctgagatgtataggtaacctgtgatctcttccagagtctcaaacacttggagctgctctggctggagcggggcagtgttggaggctgggtccccatcaaagctctccggcagaaatgccaggctcccaaagatcttcttgcagccagcaaactcctggatattcttccacaaaatcgtgtcctggtagcagagctgggggttccgctggatcaagacccctcctttcaagatctctgtgaggcttcgaagctgcagctcccgcaggcctcctggggaggcccctgtgacaggggtggtattgttcagcgggtctccattgtctagcacggccagggcatagttgtcctcaaagagctgggtgcctcgcacaatccgcagcctctgcagtgggacctgcctcacttggttgtgagcgatgagcacgtagccctgcacctcctggatatcctgcaggaaggacaggctggcattggtgggcaggtaggtgagttccaggtttccctgcaccacctggcagccctggtagaggtggcggagcatgtccaggtgggttctagatttatcacgtacccatttccccgcatcttttatttttttaaatactttagggaaaaatggtttttgatttgcttttaaaggttgtggtgtagactcgtctgctgactgcatgctagaatctaagtcactttcagaagcatccacaactgactctttcgccacttttctcttatttgcttttgttggtttatctggataagtaaggctttcaagctcactatccgacgacgctatggcttttcttctttttttaatttccgctgcgctatccgatgacagacctggatgacgacgctccacttgcagagttggtcggtcgactcctgaagcctcttcatttatagccacatttcctgtttgctcaccgttgttattattgttattcggacctttctctgcttttgctttcaacattgctattaggtctgctttgttcgtatttttcactttattcgatttttctagttcctcaatatcacgtgaacttacttcacgtgcagtttcgtatcttggtcccgtatttacctcgcttggctgctcttctgttttttcttcttcccattcatctgtgtttagactggaatcttcgctatctgtcgctgcaaatattatgtcggggttaatcgtaatgcagttggcagtaatgaaaactaccatcatcgcacgcataaatctgtttaatcccacttatactccctcctcgtgatacgctaatacaacctttttagaacaaggaaaattcggccttcattttcactaatttgttccgttaaaaattggattagcagttagttatcttcttaattagctaatataagaaaaaatattcatgaattattttaagaatatcacttggagaattaatttttctctaacatttgttaatcagttaaccccaactgcttcccaagcttcacccgggccactaactcaacgctagtagtggatttaatcccaaatgagccaacagaaccagaaccagaaacagaacaagtaacattggagttagaaatggaagaagaaaaaagcaatgatttcgtgtgaataatgcacgaaatcattgcttatttttttaaaaagcgatatactagatataacgaaacaacgaactgaataaagaatacaaaaaaagagccacgaccagttaaagcctgagaaactttaactgcgagccttaattgattaccaccaatcaattaaagaagtcgagacccaaaatttggtaaagtatttaattactttattaatcagatacttaaatatctgtaaacccattatatcgggtttttgaggggatttcaagtctttaagaagataccaggcaatcaattaagaaaaacttagttgattgccttttttgttgtgattcaactttgatcgtagcttctaactaattaattttcgtaagaaaggagaacagctgaatgaatatcccttttgttgtagaaactgtgcttcatgacggcttgttaaagtacaaatttaaaaatagtaaaattcgctcaatcactaccaagccaggtaaaagtaaaggggctatttttgcgtatcgctcaaaaaaaagcatgattggcggacgtggcgttgttctgacttccgaagaagcgattcacgaaaatcaagatacatttacgcattggacaccaaacgtttatcgttatggtacgtatgcagacgaaaaccgttcatacactaaaggacattctgaaaacaatttaagacaaatcaataccttctttattgattttgatattcacacggaaaaagaaactatttcagcaagcgatattttaacaacagctattgatttaggttttatgcctacgttaattatcaaatctgataaaggttatcaagcatattttgttttagaaacgccagtctatgtgacttcaaaatcagaatttaaatctgtcaaagcagccaaaataatctcgcaaaatatccgagaatattttggaaagtctttgccagttgatctaacgtgcaatcattttgggattgctcgtataccaagaacggacaatgtagaattttttgatcccaattaccgttattctttcaaagaatggcaagattggtctttcaaacaaacagataataagggctttactcgttcaagtctaacggttttaagcggtacagaaggcaaaaaacaagtagatgaaccctggtttaatctcttattgcacgaaacgaaattttcaggagaaaagggtttagtagggcgcaatagcgttatgtttaccctctctttagcctactttagttcaggctattcaatcgaaacgtgcgaatataatatgtttgagtttaataatcgattagatcaacccttagaagaaaaagaagtaatcaaaattgttagaagtgcctattcagaaaactatcaaggggctaatagggaatacattaccattctttgcaaagcttgggtatcaagtgatttaaccagtaaagatttatttgtccgtcaagggtggtttaaattcaagaaaaaaagaagcgaacgtcaacgtgttcatttgtcagaatggaaagaagatttaatggcttatattagcgaaaaaagcgatgtatacaagccttatttagcgacgaccaaaaaagagattagagaagtgctaggcattcctgaacggacattagataaattgctgaaggtactgaaggcgaatcaggaaattttctttaagattaaaccaggaagaaatggtggcattcaacttgctagtgttaaatcattgttgctatcgatcattaaattaaaaaaagaagaacgagaaagctatataaaggcgctgacagcttcgtttaatttagaacgtacatttattcaagaaactctaaacaaattggcagaacgccccaaaacggacccacaactcgatttgtttagctacgatacaggctgaaaataaaacccgcactatgccattacatttatatctatgatacgtgtttgtttttctttgctggctagcttaattgcttatatttacctgcaataaaggatttcttacttccattatactcccattttccaaaaacatacggggaacacgggaacttattgtacaggccacctcatagttaatggtttcgagccttcctgcaatctcatccatggaaatatattcatccccctgccggcctattaatgtgacttttgtgcccggcggatattcctgatccagctccaccataaattggtccatgcaaattcggccggcaattttcaggcgttttcccttcacaaggatgtcggtccctttcaattttcggagccagccgtccgcatagcctacaggcaccgtcccgatccatgtgtctttttccgctgtgtactcggctccgtagctgacgctctcgccttttctgatcagtttgacatgtgacagtgtcgaatgcagggtaaatgccggacgcagctgaaacggtatctcgtccgacatgtcagcagacgggcgaaggccatacatgccgatgccgaatctgactgcattaaaaaagccttttttcagccggagtccagcggcgctgttcgcgcagtggaccattagattctttaacggcagcggagcaatcagctctttaaagcgctcaaactgcattaagaaatagcctctttctttttcatccgctgtcgcaaaatgggtaaatacccctttgcactttaaacgagggttgcggtcaagaattgccatcacgttctgaacttcttcctctgtttttacaccaagtctgttcatccccgtatcgaccttcagatgaaaatgaagagaaccttttttcgtgtggcgggctgcctcctgaagccattcaacagaataacctgttaaggtcacgtcatactcagcagcgattgccacatactccgggggaaccgcgccaagcaccaatataggcgccttcaatccctttttgcgcagtgaaatcgcttcatccaaaatggccacggccaagcatgaagcacctgcgtcaagagcagcctttgctgtttctgcatcaccatgcccgtaggcgtttgctttcacaactgccatcaagtggacatgttcaccgatatgttttttcatattgctgacattttcctttatcacggacaagtcaatttccgcccacgtatctctgtaaaaaggttttgtgctcatggaaaactcctctcttttttcagaaaatcccagtacgtaattaagtatttgagaattaattttatattgattaatactaagtttacccagttttcacctaaaaaacaaatgatgagataatagctccaaaggctaaagaggactataccaactatttgttaat

SEQ ID NO: 32 - protein, 10 aa, Artificial Sequence
  PSA H-2Db peptide
HCIRNKSVIL

SEQ ID NO: 33 - protein, 9 aa, Artificial Sequence
  HPV16 E7 H-2Db
RAHYNIVTF

SEQ ID NO: 34 - DNA, 19 bp, Artificial Sequence
  primer 3 (Adv 305)  that binds externally to the actA deletion region
tgggatggccaagaaattc

SEQ ID NO: 35 - DNA, 22 bp, Artificial Sequence
  primer 4 (Adv 304) that binds externally to the actA deletion region
ctaccatgtcttccgttgcttg